(19) United States
(12) Patent Application Publication
(10) Pub. No.: US 2014/0315738 A1
(43) Pub. Date: Oct. 23, 2014

(54) MARINE MEDAKA GENES RESPONDING TO THE EXPOSURE OF ENDOCRINE-DISRUPTING CHEMICALS, AND METHOD FOR DIAGNOSING AN AQUATIC ECO-SYSTEM CONTAMINATION USING SAME

(71) Applicant: Korea Institute of Ocean Science & Technology, Gyeonggi-do (KR)

(72) Inventors: Seungshic Yum, Gyeongsangnam-do (KR); Seonock Woo, Gyeongsangnam-do (KR); Hyokyoung Won, Gyeongsangnam-do (KR); Aekyung Lee, Busan (KR); Jae-Chun Ryu, Seoul (KR)

(73) Assignee: Korea Ocean Research And Development Institute, Gyeonggi-do (KR)

(21) Appl. No.: 14/256,609

(22) Filed: Apr. 18, 2014

Related U.S. Application Data
(63) Continuation of application No. PCT/KR2012/002105, filed on Mar. 23, 2012.

(30) Foreign Application Priority Data
Oct. 20, 2011 (KR) 10-2011-0107570
Feb. 28, 2012 (KR) 10-2012-0020191

(51) Int. Cl. C12Q 1/68

(52) U.S. Cl. CPC C12Q 1/6883
USPC 506/9; 506/17

(57) ABSTRACT

The present invention relates to marine medaka genes responding to the exposure of endocrine-disrupting chemicals, and a method for diagnosing an aquatic eco-system contamination using the marine medaka genes. Specifically, the method for diagnosing an aquatic eco-system contamination, according to the present invention, makes use of marine medaka genes of which the expression amount changes according to 17β-estradiol, or marine medaka genes of which the expression amount changes according to bisphenol A. Since it was verified that genes of marine medaka exposed to 17β-estradiol are changed or genes of marine medaka exposed to bisphenol A are changed, the present invention can be used in a useful manner for a microarray chip in which marine medaka genes responding to 17β-estradiol or marine medaka genes responding to bisphenol A are integrated, for a diagnosis using same, and for a method for diagnosing health or detecting stress of a marine eco-system by applying a kit having the microarray chip.

### CROSS REFERENCE TO RELATED APPLICATIONS

This is a continuation of International Patent Application No. PCT/KR2012/002105, filed Mar. 23, 2012, which claims the benefit of Korean Patent Application No. 10-2012-0020191, filed Feb. 28, 2012, and Korean Patent Application No. 10-2011-0107570, filed Oct. 20, 2011, all of which are incorporated by reference herein in their entirety.

### SEQUENCE LISTING

The Sequence Listing is submitted as an ASCII text file in the form of the file named Squence_Listing.txt, which was created on Apr. 17, 2014, and is 198,333 bytes, which is incorporated by reference herein.

### FIELD OF THE INVENTION

The present invention relates to genes of Javanese medaka (Oryzias Javanicus) differentially regulated upon exposure to endocrine-disrupting chemicals and methods using the same for diagnosing environmental pollution of hydroecosystem.

### BACKGROUND OF THE INVENTION

Endocrine disrupting chemicals (EDC) or endocrine disruptors (ED) are the chemicals that interfere with hormone system of a living organism exposed thereto. The EDs include dioxin, PCB, PAH, furan, or phenol, for example.

Among these, 17β-estradiol (E2) is known as one of the strongest estrogen-family steroid hormone (estrone (E1), 17β-estradiol and estriol (E3)). In humans, E2 is prevalently formed in the ovaries and placentas of women of childbearing age, but also observed in fat tissues of men or menopausal women. 17β-estradiol in synthesized form is used for regulating a variety of menopause-associated symptoms such as vasomotor dysfunction, vulvar and vaginal contraction disorders, and osteoporosis. Estron and estriol including 17β-estradiol released from humans and animals are distributed to environment via sewage treatment plants or animal waste. Accordingly, the safety of ecosystem is threatened by the exposures to the steroid hormones. For example, the hormones induce vitellogenin synthesis and feminization of male fish, which can possibly affect normal endocrine functions and male fertility and subsequently disturb ecosystem. Accordingly, a method is necessary, which can estimate presence/absence of 17β-estradiol, predict biological changes due to exposure thereto, and make early diagnosis.

Bisphenol A (BPA) is another example of EDs. BPA has been widely used for the manufacture of plastic products since 1950, and also used for the manufacture of epoxy resin which is applied on inner sides of food cans. Food preserved in BPA containers is the major route of human health exposure to BPA. The general understanding is that the amount of BPA release particularly increases when the container surface becomes damaged or heated. BPA is directly distributed by sewage water from the plastic manufacturing factories or domestic sewage, or leaches from the plastic garbage disposed in environment.

Exposure to BPA is understood to cause adverse effects on growth, reproductive health and development of aquatic organisms. Endocrine disruption effect was observed in invertebrates, fish, amphibians and reptiles at actual concentrations of environment without acute toxicity. Researches in Canada revealed that certain fish groups in non-contaminated water system had 55% females, whereas the female proportion was 85% in the water system that is contaminated with EDs including BPA. The above suggests that EDs destruct biological population ratio, thus causing severe disruption in ecosystem. Along with estrogen, BPA is known as an ED of living organisms, which impedes generation and development, and induces attention deficit hyperactivity disorder due to disturbance of nervous system, deformity, and abnormal gene expression due to inhibited DNA methylation. The chemical is also reported to stimulate development of a variety of cancerous cells. In September, 2010, Canada declared, for the first time, BPA on the toxic substance list and the chemical is currently banned for use in infant feeding bottles in the European Union (EU) and Canada. It is thus necessary to develop a method, with which it is possible to determine presence/absence of BPA in hydroecosystem, estimate biological changes from exposure to the chemical, and make early diagnosis.

Living organisms have developed biological defense mechanisms to maintain homeostasis in response to changing external factors such as pollutions, atmosphere or infection with pathogenic microorganisms. The biological defense mechanisms involve actively changing expression levels of specific genes and regulating amounts of specific proteins. This suggests that, by investigating the genes that are specific to certain changes and monitoring their expression levels, it will be possible to obtain not only information about environmental changes of a specific area, but also information about the influence that such environment changes can have on phenomenon of life and health of ecosystem.

To be specific, the Javanese medaka (oryzias javanicus) (Inoue and Takei, Zoological Sci., 19, 727-734, 2002), which is highly adaptable to all levels of salinities (i.e., to both fresh water and seawater), is expected to be applicable for the researches on environmental risk assessment by chemical pollutants. The following researches have been conducted on changes in proteins and gene expressions of Javanese medaka in response to toxic chemicals: research on fertility and vitellogenin protein concentration variations of male Javanese medaka liver tissues exposed to 17β estradiol (E2) (Imai et al., Mar. Poll. Bull., 51, 708-714, 2005); research on choriogenin H and L gene expression level differences in male Javanese medaka liver tissues in response to exposure to E2 (Yu et al., Aquat. Toxicol., 77, 348-358, 2006); research on metallothionein gene expression level differences in Javanese medaka liver tissues in response to exposure to a variety of heavy metals (Woo et al., Mar. Biotech., 8, 654-662, 2006); research on fertility and vitellogenin protein concentration differences in Javanese medaka liver tissues in response to exposure to estrone (E1) in environment (Imai et al., Environ. Toxicol. Chem., 26, 726-731, 2007). Additional researches reported expression level differences of antioxidant enzymes and stress-associated genes in response to exposure to six major heavy metals (Ag, Cd, Cr, Cu, Ni, Zn) (Woo et al., Comp. Biochem. Physiol. C, 149, 289-299, 2009); organophosphorus pesticides (Iprobenfos) (Woo et al., Comp. Biochem. Physiol. C, 149, 427-432, 2009); and polychlorinated camphene (Toxaphene) (Woo et al., Comp. Biochem. Physiol. C, 153, 355-361, 2011). However, none has confirmed gene expression differences of Javanese medaka in response to exposure to 17β-estradiol or BPA, to use this as a biomarker to detect possible exposure to EDC, i.e., 17β-estradiol or BPA.

The present inventors have investigated biomarkers that react to marine environmental pollution such as exposure to EDCs with Javanese medaka by, specifically, investigating biomarkers that can confirm possible exposure to 17β-estradiol and expression level differences of these gene biomarkers, and also investigating biomarkers that can confirm possible exposure to BPA and expression level differences of these gene biomarkers, and as a result, completed the present invention based on the confirmation that the biomarkers can be applied for the diagnosis of environmental pollution in hydroecosystem.

### DETAILED DESCRIPTION OF THE INVENTION

### Technical Problem

It is one object of the present invention to provide a DNA microarray chip and a method using the same for detecting exposure to 17β-estradiol, a kind of endocrine disrupting chemicals and diagnosing environmental pollution in a hydroecosystem.

Also, it is another object of the present invention to provide a DNA microarray chip and a method using the same for detecting exposure to Bisphenol A (BPA), a kind of endocrine disrupting chemicals and diagnosing environmental pollution in a hydroecosystem.

### Technical Solution

To achieve said object, the present invention provides a DNA microarray chip for detecting exposure to 17β-estradiol and diagnosing environmental pollution, wherein said DNA microarray chip comprises the full-length cDNAs, the partial oligonucleotides fragment or their complementary oligonucleotides equivalents of at least one gene of Javanese medaka (Oryzias javanicus) which is increased or decreased upon exposure to 17β-estradiol.

The present invention also provides a method using said gene for detecting exposure to 17β-estradiol and diagnosing environmental pollution.

The present invention also provides a kit comprising said DNA microarray chip for detecting exposure to 17β-estradiol and diagnosing environmental pollution.

The present invention also provides a kit comprising complementary primer pairs to amplify said gene for detecting stress caused by exposure to 17β-estradiol and diagnosing environmental pollution.

The present invention also provides a use of gene of which expression is changed upon exposure to 17β-estradiol to produce a DNA microarray chip for detecting exposure to 17β-estradiol and diagnosing environmental pollution.

The present invention also provides a use of gene of which expression is changed upon exposure to 17β-estradiol for producing a kit for detecting exposure to 17β-estradiol and diagnosing environmental pollution.

The present invention also provides a DNA microarray chip for detecting exposure to Bisphenol A (BPA) and diagnosing environmental pollution, wherein said DNA microarray chip comprises the full-length cDNAs, the partial oligonucleotides fragment or their complementary oligonucleotides equivalents of at least one gene of Javanese medaka (Oryzias javanicus) which are increased or decreased upon exposure to Bisphenol A.

The present invention also provides a method using said gene for detecting exposure to Bisphenol A and diagnosing environmental pollution.

The present invention also provides a kit comprising complementary primer pairs to amplify said gene for detecting stress caused by exposure to Bisphenol A and diagnosing environmental pollution.

The present invention also provides a use of gene of which expression is changed upon exposure to Bisphenol A for producing a DNA microarray chip for detecting exposure to Bisphenol A and diagnosing environmental pollution.

The present invention also provides a use of gene of which expression is changed upon exposure to Bisphenol A for producing a kit for detecting exposure to Bisphenol A and diagnosing environmental pollution.

### Advantageous Effect

A method for diagnosing environmental pollution in hydroecosystem according to the present invention is using genes of Javanese medaka (Oryzias javanicus) of which expressions are changed upon exposure to 17β-estradiol or Bisphenol A. Specifically, since increased or decreased genes were identified in Javanese medaka (Oryzias javanicus) exposed to 17β-estradiol or Bisphenol A, a microarray chip comprising those genes, a diagnosing method using those chips and a kit comprising those chips can be usefully exploited for the detection of stress and health examination in marine ecosystem. Furthermore, a diagnosing method using complementary primer pairs to amplify genes on the microarray chip and a kit comprising the same things can be usefully exploited for the detection of stress and health examination in marine ecosystem

### BEST MODE

Hereinafter, the present invention is described in detail.

In the present invention, genes, especially those which are closely involved in defense mechanisms against external stress were identified of which expressions were changed upon exposure to 17β-estradiol, a kind of endocrine-disrupting chemicals or Bisphenol A. Therefore, a DNA microarray chip comprising differentially regulated at least one gene of Javanese medaka (Oryzias javanicus) upon exposure to 17β-estradiol or Bisphenol A can be exploited for detecting stress and diagnosing the pollution of hydroecosystem from a specimen exposed to 17β-estradiol or Bisphenol A.

For detecting exposure to 17β-estradiol and diagnosing environmental pollution from a specimen, the present invention provides at least one gene selected from the group consisting of Dimethylglycine dehydrogenase (SEQ ID NO:1), Fructose-bisphosphate aldolase B (SEQ ID NO: 2), Fatty acid binding protein 10 liver basic (SEQ ID NO:3), Claudin (SEQ ID NO:4), Cytochrome P450 2P3 (SEQ ID NO:5), Aldolase B (SEQ ID NO:6), Cytochrome c-1, cyc1 (SEQ ID NO:7), Selenoprotein M (SEQ ID NO:8), ATPase H+ transporting V1 subunit F, atp6v1f (SEQ ID NO:9), Cytochrome oxidase subunit I, COI (SEQ ID NO:10), ATP citrate lyase isoform 2 (SEQ ID NO:11), Ribosomal protein L13a, rpl13a (SEQ ID NO:12), Cytochrome c oxidase subunit I (SEQ ID NO:13), Pyrroline-5-carboxylate reductase 1, pycr1 (SEQ ID NO:14), Exs-related protein (SEQ ID NO:15), Cysteine-rich with EGF-like domains 2, creld2 (SEQ ID NO:16), Selenoprotein 15 (SEQ ID NO:17), Beta-galactoside-binding lectin (SEQ ID NO:18), hv1 gene for histone H2 variant (SEQ ID NO:19), LAG1 longevity assurance 2 (SEQ ID NO:20), Inositol oxygenase (SEQ ID NO:21), Acyl-CoA synthetase long-chain family member 1 (SEQ ID NO:22), Tetraspanin-3 (SEQ ID NO:23), Microsomal triglyceride transfer protein (SEQ ID NO:24), Amino-terminal enhancer of split (SEQ ID NO:25), non-classical MHC class I antigen (SEQ ID NO:26), NADH-cytochrome b5 reductase 3 (SEQ ID NO:27), rRNA2-O-methyltransferase fibrillarin (SEQ ID NO:28), Vitellogenin II, vit-6 (SEQ ID NO:29), Choriogenin H minor (SEQ ID NO:30), Adenylosuccinate synthetase isozyme 2 (SEQ ID NO:31), Mannose-P-dolichol utilization defect 1b, mpdu1b (SEQ ID NO:32), RAB3 GTPase activating protein subunit 2 (non-catalytic)(SEQ ID NO:33), Tax1 (human T-cell leukemia virus type I) binding protein 1b (SEQ ID NO:34), 40S ribosomal protein S24-like protein (SEQ ID NO:35), Exosome complex exonuclease RRP4 (SEQ ID NO:36), Iduronate 2-sulfatase precursor (SEQ ID NO:37), Vitellogenin 1, (SEQ ID NO:38), Oligosaccharyltransferase complex subunit (SEQ ID NO:39), Abhydrolase domain containing 11 (SEQ ID NO:40), B-cell antigen receptor complex-associated protein alpha-chain precursor (SEQ ID NO:41), Alpha-2,3-sialyltransferase ST3Gal I (SEQ ID NO:42), mitochondrial Glutathione reductase (SEQ ID NO:43), Choriogenin L (SEQ ID NO:44), Integral membrane protein 1 (SEQ ID NO:45), Complement control protein factor I-B (SEQ ID NO:46), Glutamate receptor, ionotropic, delta 1, GRID1 (SEQ ID NO:47),

Dolichyl-alpha-1,6-mannosyltransferase (alg12) (SEQ ID NO:48), Macrosialin precursor (SEQ ID NO:49), Metalloreductase STEAP4 (SEQ ID NO:50), 14-alpha demethylase (CYP51) (SEQ ID NO:51), Bromodomain containing 2 (RING3) (SEQ ID NO:52), Apolipoprotein B (SEQ ID NO:53), Glutamate dehydrogenase 1b (SEQ ID NO:54), Glucose-6-phosphate dehydrogenase (SEQ ID NO:55) and Transferrin (SEQ ID NO:56).

Said gene of which expression is increased upon exposure to 17β-estradiol can be selected from the following group but not limited thereto:

Cytochrome c-1, cyc1 (SEQ ID NO:7), Selenoprotein M (SEQ ID NO:8), ATPase H+ transporting V1 subunit F, atp6v1f (SEQ ID NO:9), Cytochrome oxidase subunit I, COI (SEQ ID NO:10), ATP citrate lyase isoform 2 (SEQ ID NO:11), Ribosomal protein L13a, rpl13a (SEQ ID NO:12), Cytochrome c oxidase subunit I (SEQ ID NO:13), Pyrroline-5-carboxylate reductase 1, pycr1 (SEQ ID NO:14), Exs-related protein (SEQ ID NO:15), Cysteine-rich with EGF-like domains 2, creld2 (SEQ ID NO:16), Selenoprotein 15 (SEQ ID NO:17), Beta-galactoside-binding lectin (SEQ ID NO:18), hv1 gene for histone H2 variant (SEQ ID NO:19), LAG1 longevity assurance 2 (SEQ ID NO:20), Inositol oxygenase (SEQ ID NO:21), Acyl-CoA synthetase long-chain family member 1 (SEQ ID NO:22), Tetraspanin-3 (SEQ ID NO:23), Microsomal triglyceride transfer protein (SEQ ID NO:24), Amino-terminal enhancer of split (SEQ ID NO:25), non-classical MHC class I antigen (SEQ ID NO:26), NADH-cytochrome b5 reductase 3 (SEQ ID NO:27), rRNA 2-O-methyltransferase fibrillarin (SEQ ID NO:28), Vitellogenin II, vit-6 (SEQ ID NO:29), Choriogenin H minor (SEQ ID NO:30), Adenylosuccinate synthetase isozyme 2 (SEQ ID NO:31), Mannose-P-dolichol utilization defect 1b, mpdu1b (SEQ ID NO:32), RAB3 GTPase activating protein subunit 2 (non-catalytic) (SEQ ID NO:33), Tax1 (human T-cell leukemia virus type I) binding protein 1b (SEQ ID NO:34), 40S ribosomal protein S24-like protein (SEQ ID NO:35), Exosome complex exonuclease RRP4 (SEQ ID NO:36), Iduronate 2-sulfatase precursor (SEQ ID NO:37), Vitellogenin 1, (SEQ ID NO:38), B-cell antigen receptor complex-associated protein alpha-chain precursor (SEQ ID NO:41), Alpha-2,3-sialyltransferase ST3Gal I (SEQ ID NO:42), mitochondrial Glutathione reductase (SEQ ID NO:43), Choriogenin L (SEQ ID NO:44), Integral membrane protein 1 (SEQ ID NO:45), Complement control protein factor I-B (SEQ ID NO:46), Glutamate receptor, ionotropic, delta 1, GRID1 (SEQ ID NO:47),

Dolichyl-alpha-1,6-mannosyltransferase (alg12) (SEQ ID NO:48), Macrosialin precursor (SEQ ID NO:49), Metalloreductase STEAP4 (SEQ ID NO:50), 14-alpha demethylase (CYP51) (SEQ ID NO:51), Bromodomain containing 2 (RING3) (SEQ ID NO:52).

Said gene of which expression is decreased in response to exposure to 17β-estradiol can be selected from the following group but not limited thereto:

Dimethylglycine dehydrogenase (SEQ ID NO:1), Fructose-bisphosphate aldolase B (SEQ ID NO:2), Fatty acid binding protein 10 liver basic (SEQ ID NO:3), Claudin (SEQ ID NO:4), Cytochrome P450 2P3 (SEQ ID NO:5), Aldolase B (SEQ ID NO:6), Oligosaccharyltransferase complex subunit (SEQ ID NO:39) and Abhydrolase domain containing 11 (SEQ ID NO:40).

Said microarray chip can comprise the full-length cDNAs (complementary DNAs), the partial oligonucleotides fragments or its complementary oligonucleotides equivalents of at least one gene involved in defense mechanisms against external stress selected from the following group but not limited thereto:

Cytochrome P450 2P3 (SEQ ID NO:5), Selenoprotein M (SEQ ID NO:8), Vitellogenin 1, (SEQ ID NO:38), Apolipoprotein B (SEQ ID NO:53), Glutamate dehydrogenase 1b (SEQ ID NO:54), Glucose-6-phosphate dehydrogenase (SEQ ID NO:55) and Transferrin (SEQ ID NO:56).

Said gene can be originated from Javanese medaka (Oryzias javanicus) but not limited thereto.

Said microarray chip can detect a reaction of Javanese medaka (Oryzias javanicus) in response to 17β-estradiol pollution but not limited thereto.

To explore genes of Oryzias javanicus differentially regulated upon exposure to 17β-estradiol, Oryzias javanicus was exposed to 17β-estradiol in a concentration of 100 μg/L for 24 or 48 hour and then cDNAs were synthesized and genes showing changed expressions were investigated. Specifically, cDNAs were synthesized from mRNAs extracted from Javanese medaka (Oryzias javanicus) reared in seawater of the absence or presence of 17β-estradiol. PCR was performed to amplify fragments of genes of which expressions were changed. As the result, 56 genes of which expressions were increased or decreased were selected (Refer to table 1 or table 2). Steroid hormones of estrogen series like 17β-estradiol result in the disturbance of ecosystem, impairing the normal endocrine function and the followed sexual function which are attributable to the synthesis of vitellogenin and feminization in males. Therefore, there can be investigated stress and health of marine organisms according to alterations of external environment by analyzing expression profiles of genes causing physiological and metabolic changes. Also, those genes can be exploited as biomarkers for diagnosing health of living things and the environment.

Therefore, for detecting stress and diagnosing conditions resulting from pollution by persistent toxic substances like 17β-estradiol, said gene can be usefully exploited as biomarkers and in a microarray chip since expression of said gene showed significant difference upon exposure to 17β-estradiol.

The present invention also provides a method for detecting exposure to 17β-estradiol and diagnosing environmental pollution, said method comprising the steps of:

1) separating RNAs from Oryzias Javanicus of a sample of an experimental group exposed to a specimen, and from Oryzias Javanicus of a control group;

2) converting RNAs extracted from the experimental group and the control group of step 1) into cDNA and labeling them with different fluorescent materials;

3) hybridizing cDNAs labeled with different fluorescent materials of step b) to said microarray chip;

4) analyzing the reacted microarray chip of step 3); and

5) comparing expression levels of genes on the microarray chip between the experimental group and the control group.

In said method, fluorescent materials of step 2) can be selected from the group consisting of Cy3, Cy5, poly L-lysine-fluorescein isothiocyanate (FITC), rhodamine-B-isothiocyanate (RITC) and rhodamine but not limited thereto and all fluorescent materials known to a person skilled in the art can be used.

For the microarray chip of step 4) in said method, any microarray chip can be used if it contains said gene. Above procedures are preferably performed according to conventional experimental protocols of microarray chip but not limited thereto:

The present invention also provides a method for detecting exposure to 17β-estradiol (E2) and diagnosing environmental pollution, said method comprising the steps of:

1) separating RNAs from Oryzias Javanicus of a sample of an experimental group exposed to a specimen (17β-estradiol), and from Oryzias Javanicus of a control group;

2) performing real time RT-PCR with the RNAs of step b) using a primer pair complementary to said gene to amplify it; and

3) comparing gene expression levels between the experimental group and the control group as measured by real-time RT-PCR in step 2).

For a primer pair of step 2) in said method, any primer pair can be used if it is complementary to a selected gene and can amplify said gene.

The present inventors found that they can diagnose environmental conditions caused by exposure to 17β-estradiol through the use of said gene, uncovering 56 genes of which expressions are increased or decreased in response to exposure to 17β-estradiol. Therefore, in the present invention genes differentially regulated upon exposure 17β-estradiol can be usefully exploited for said microarray chip as well as a stress detection and a diagnosis of environmental pollution using real-time RT-PCR.

The present invention also provides a kit comprising said microarray chip for detecting exposure to 17β-estradiol and diagnosing environmental pollution.

Said kit can comprise Javanese medaka (Oryzias Javanicus) additionally.

Said kit can additionally comprise one selected from the group consisting of strepavidin-like phosphatease conjugate, chemifluorescent materials and chemiluminescent materials additionally but not limited thereto.

Said kit can additionally comprise one selected from the group of reactive reagents consisting of hybridization buffer, reverse transcriptase for the synthesis of cDNA from RNA, dNTPs, rNTP, labeling reagent and washing buffer but not limited thereto.

The present inventors found that they can diagnose environmental conditions caused by exposure to 17β-estradiol through the use of said gene, uncovering 56 genes of which expressions are increased or decreased in response to exposure to 17β-estradiol. Therefore, in the present invention genes differentially regulated upon exposure 17β-estradiol can be usefully exploited for said kit comprising said microarray chip for detecting stress and diagnosing environmental pollution.

The present invention also provides a kit comprising a complementary primer pair to amplify said gene on the microarray chip for detecting stress and diagnosing environmental pollution.

Said primer pair is complementary to said gene and can amplify it and all forward and reverse primer pairs designed to yield 100 to 300 bp products can be used.

Said kit can comprise Javanese medaka (Oryzias javanicus) additionally.

Said kit can additionally comprise at least one selected from the group of reactive reagents consisting of reverse transcriptase for the synthesis of cDNA from RNA, cNTPs, rNTP, DNA polymerase and washing buffer but not limited thereto.

To produce said microarray chip for detecting exposure to 17β-estradiol and diagnosing environmental pollution from a specimen, the present invention also provides a use of a gene which expression is differentially regulated upon exposure to 17β-estradiol selected from the group consisting of Cytochrome c-1, cyc1 (SEQ ID NO:7), Selenoprotein M (SEQ ID NO:8), ATPase H+ transporting V1 subunit F, atp6v1f (SEQ ID NO:9), Cytochrome oxidase subunit I, COI (SEQ ID NO:10), ATP citrate lyase isoform 2 (SEQ ID NO:11), Ribosomal protein L13a, rpl13a (SEQ ID NO:12), Cytochrome c oxidase subunit I (SEQ ID NO:13), Pyrroline-5-carboxylate reductase 1, pycr1 (SEQ ID NO:14), Exs-related protein (SEQ ID NO:15), Cysteine-rich with EGF-like domains 2, creld2 (SEQ ID NO:16), Selenoprotein 15 (SEQ ID NO:17), Beta-galactoside-binding lectin (SEQ ID NO:18), hv1 gene for histone H2 variant (SEQ ID NO:19), LAG1 longevity assurance 2 (SEQ ID NO:20), Inositol oxygenase (SEQ ID NO:21), Acyl-CoA synthetase long-chain family member 1 (SEQ ID NO:22), Tetraspanin-3 (SEQ ID NO:23), Microsomal triglyceride transfer protein (SEQ ID NO:24), Amino-terminal enhancer of split (SEQ ID NO:25), non-classical MHC class I antigen (SEQ ID NO:26), NADH-cytochrome b5 reductase 3 (SEQ ID NO:27), rRNA 2-O-methyltransferase fibrillarin (SEQ ID NO:28), Vitellogenin II, vit-6 (SEQ ID NO:29), Choriogenin H minor (SEQ ID NO:30), Adenylosuccinate synthetase isozyme 2 (SEQ ID NO:31), Mannose-P-dolichol utilization defect 1b, mpdu1b (SEQ ID NO:32), RAB3 GTPase activating protein subunit 2 (non-catalytic) (SEQ ID NO:33), Tax1 (human T-cell leukemia virus type I) binding protein 1b (SEQ ID NO:34), 40S ribosomal protein S24-like protein (SEQ ID NO:35), Exosome complex exonuclease RRP4 (SEQ ID NO:36), Iduronate 2-sulfatase precursor (SEQ ID NO:37), Vitellogenin 1, (SEQ ID NO:38), B-cell antigen receptor complex-associated protein alpha-chain precursor (SEQ ID NO:41), Alpha-2,3-sialyltransferase ST3Gal I (SEQ ID NO:42), mitochondrial Glutathione reductase (SEQ ID NO:43), Choriogenin L (SEQ ID NO:44), Integral membrane protein 1 (SEQ ID NO:45), Complement control protein factor I-B (SEQ ID NO:46), Glutamate receptor, ionotropic, delta 1, GRID1 (SEQ ID NO:47),

Dolichyl-alpha-1,6-mannosyltransferase (alg12) (SEQ ID NO:48), Macrosialin precursor (SEQ ID NO:49), Metalloreductase STEAP4 (SEQ ID NO:50), 14-alpha demethylase (CYP51) (SEQ ID NO:51), Bromodomain containing 2 (RING3) (SEQ ID NO:52), Apolipoprotein B (SEQ ID NO:53), Glutamate dehydrogenase 1b (SEQ ID NO:54), Glucose-6-phosphate dehydrogenase (SEQ ID NO:55) and Transferrin (SEQ ID NO:56).

Furthermore, to produce said kit for detecting exposure to 17β-estradiol and diagnosing environmental pollution from a specimen, the present invention also provides a use of a gene which expression is differentially regulated upon exposure to 17β-estradiol selected from the group consisting of Cytochrome c-1, cyc1 (SEQ ID NO:7), Selenoprotein M (SEQ ID NO:8), ATPase H+ transporting V1 subunit F, atp6v1f (SEQ ID NO:9), Cytochrome oxidase subunit I, COI (SEQ ID NO:10), ATP citrate lyase isoform 2 (SEQ ID NO:11), Ribosomal protein L13a, rpl13a (SEQ ID NO:12), Cytochrome c oxidase subunit I (SEQ ID NO:13), Pyrroline-5-carboxylate reductase 1, pycr1 (SEQ ID NO:14), Exs-related protein (SEQ ID NO:15), Cysteine-rich with EGF-like domains 2, creld2 (SEQ ID NO:16), Selenoprotein 15 (SEQ ID NO:17), Beta-galactoside-binding lectin (SEQ ID NO:18), hv1 gene for histone H2 variant (SEQ ID NO:19), LAG1 longevity assurance 2 (SEQ ID NO:20), Inositol oxygenase (SEQ ID NO:21), Acyl-CoA synthetase long-chain family member 1 (SEQ ID NO:22), Tetraspanin-3 (SEQ ID NO:23), Microsomal triglyceride transfer protein (SEQ ID NO:24), Amino-terminal enhancer of split (SEQ ID NO:25), non-classical MHC class I antigen (SEQ ID NO:26), NADH-cytochrome b5 reductase 3 (SEQ ID NO:27), rRNA 2-O-methyltransferase fibrillarin (SEQ ID NO:28), Vitellogenin II, vit-6 (SEQ ID NO:29), Choriogenin H minor (SEQ ID NO:30), Adenylosuccinate synthetase isozyme 2 (SEQ ID NO:31), Mannose-P-dolichol utilization defect 1b, mpdu1b (SEQ ID NO:32), RAB3 GTPase activating protein subunit 2 (non-catalytic) (SEQ ID NO:33), Tax1 (human T-cell leukemia virus type I) binding protein 1b (SEQ ID NO:34), 40S ribosomal protein S24-like protein (SEQ ID NO:35), Exosome complex exonuclease RRP4 (SEQ ID NO:36), Iduronate 2-sulfatase precursor (SEQ ID NO:37), Vitellogenin 1, (SEQ ID NO:38), B-cell antigen receptor complex-associated protein alpha-chain precursor (SEQ ID NO:41), Alpha-2,3-sialyltransferase ST3Gal I (SEQ ID NO:42), mitochondrial Glutathione reductase (SEQ ID NO:43), Choriogenin L (SEQ ID NO:44), Integral membrane protein 1 (SEQ ID NO:45), Complement control protein factor I-B (SEQ ID NO:46), Glutamate receptor, ionotropic, delta 1, GRID1 (SEQ ID NO:47),

Dolichyl-alpha-1,6-mannosyltransferase (alg12) (SEQ ID NO:48), Macrosialin precursor (SEQ ID NO:49), Metalloreductase STEAP4 (SEQ ID NO:50), 14-alpha demethylase (CYP51) (SEQ ID NO:51), Bromodomain containing 2 (RING3) (SEQ ID NO:52), Apolipoprotein B (SEQ ID NO:53), Glutamate dehydrogenase 1b (SEQ ID NO:54), Glucose-6-phosphate dehydrogenase (SEQ ID NO:55) and Transferrin (SEQ ID NO:56).

The present inventors found that they can diagnose environmental conditions caused by exposure to 17β-estradiol through the use of said gene, uncovering 56 genes of which expressions are increased or decreased in response to exposure to 17β-estradiol. Therefore, in the present invention said primer pair to amplify genes differentially regulated upon exposure to 17β-estradiol can be usefully exploited for the kit to detect stress and diagnose environmental pollution.

The present invention also provides a microarray chip for detecting exposure to Bisphenol A from a specimen, wherein said microarray chip comprises oligonucleotides or their complementary equivalents from nucleic acid sequences of at least one gene selected from the group consisting of proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2 sex hormone-binding globulin (SEQ ID NO:152), Isocitrate dehydrogenase sex hormone-binding globulin (SEQ ID NO:153), ATP citrate lyase (ACLY), transcript variant 2 sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181).

Said gene can be originated from Javanese medaka (Oryzias javanicus) but not limited thereto.

Said gene of which expression is decreased in response to exposure to 17β-estradiol can be selected from the following group but not limited thereto:

Proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112) and DEAD-box RNA-dependent helicase p68 mRNA (SEQ ID NO:113)

Said gene of which expression is increased in response to exposure to 17β-estradiol can be selected from the following group but not limited thereto:

Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2 sex hormone-binding globulin (SEQ ID NO:152), Isocitrate dehydrogenase sex hormone-binding globulin (SEQ ID NO:153), ATP citrate lyase (ACLY), transcript variant 2sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161) and glutathione S transferase Rho-class (SEQ ID NO:162)

Said gene which is involved in the mechanism against external stress can be selected from the following group but not limited thereto:

Arylamine N-acetyl transferase (SEQ ID NO:163), Apolipoprotein E1 (SEQ ID NO:164), Basigin (SEQ ID NO:165), Complement component C8 beta (SEQ ID NO:1366), C1q-like adipose specific protein (SEQ ID NO:167), catalase (SEQ ID NO:135), Calcium binding protein P22 (SEQ ID NO:168), Ceruloplasmin (SEQ ID NO:169), complement factor B/C2-B (SEQ ID NO:131), Chitinase (SEQ ID NO:170), Choline kinase (SEQ ID NO:171), choriogenin L (SEQ ID NO:130), Delta-6 fatty acyl desaturase (SEQ ID NO:172), delta 6-desaturase (SEQ ID NO:143), Glutaminase (SEQ ID NO:173), glutathione S transferase Rho-class (SEQ ID NO:162), Hepcidin (SEQ ID NO:174), leukocyte elastase inhibitor (SEQ ID NO:110), Lipoprotein lipase (SEQ ID NO:175), N-acetyltransferase (SEQ ID NO:176), Plasminogen (SEQ ID NO:177), Retinol binding protein 4 (SEQ ID NO:178), ring finger protein 141 (SEQ ID NO:136), TBT-binding protein (SEQ ID NO:120), Transferrin (SEQ ID NO:179), uridine phosphorylase 2 (SEQ ID NO:73), Vitellogenin 1 (SEQ ID NO:180) and Warm temperature acclimation related 65 kDa protein (C243) (SEQ ID NO:181)

The present invention also provides a method for detecting exposure to Bisphenol A from a specimen, said method comprising the steps of:

1) separating RNAs from Oryzias Javanicus of a sample of an experimental group exposed to a specimen (Bisphenol A), and from Oryzias Javanicus of a control group;

2) converting RNAs extracted from the experimental group and the control group of step 1) into cDNA and labeling them with different fluorescent materials;

3) hybridizing cDNAs labeled with different fluorescent materials of step 2) to said microarray chip;

4) analyzing the reacted microarray chip of step 3); and

5) comparing expression levels of genes on the microarray chip between the experimental group and the control group.

Said specimen can be selected from the group consisting of living things, foods, chemicals, industrial samples, clinical samples and environmental samples but not limited thereto.

In said method, fluorescent materials of step 2) can be selected from the group consisting of Cy3, Cy5, poly L-lysine-fluorescein isothiocyanate (FITC), rhodamine-B-isothiocyanate (RITC) and rhodamine but not limited thereto.

The present invention also provides a method for detecting exposure to Bisphenol A from a specimen, said method comprising the steps of:

1) separating RNAs from Javanese medaka (Oryzias Javanicus) of a sample of an experimental group exposed to a specimen (Bisphenol A), and from Oryzias Javanicus of a control group;

2) performing real time RT-PCR with the RNAs of step 1) using a primer pair to amplify a gene selected from the group consisting of proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2sex hormone-binding globulin (SEQ ID NO:152), Isocitrate dehydrogenase sex hormone-binding globulin (SEQ ID NO:153), ATP citrate lyase (ACLY), transcript variant 2sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1g-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181); and

3) comparing gene expression levels between the experimental group and the control group as measured by real-time RT-PCR in step 2).

Said specimen can be selected from the group consisting of living things, foods, chemicals, industrial samples, clinical samples and environmental samples but not limited thereto.

The present invention also provides a kit comprising said microarray chip for detecting exposure to Bisphenol A from a specimen.

Said kit can additionally comprise one selected from the group consisting of strepavidin-like phosphatease conjugate, chemifluorescent materials and chemiluminescent materials additionally but not limited thereto.

Said kit can additionally comprise one selected from the group of reactive reagents consisting of reverse transcriptase for the synthesis of cDNA from RNA, cNTPs, rNTP, DNA polymerase and washing buffer but not limited thereto.

The present invention also provides a kit comprising complementary primer pairs to amplify a gene selected from the following group for detecting exposure of Bisphenol A:

Proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2sex hormone-binding globulin (SEQ ID NO:152), Isocitrate dehydrogenase sex hormone-binding globulin (SEQ ID NO:153), ATP citrate lyase (ACLY), transcript variant 2sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181).

Said primer pair can be at least one selected from the following group but not limited thereto:

Primer pair 1 (forward primer as set forth in SEQ ID NO:182 and reverse primer as set forth in SEQ ID NO:183), primer pair 2 (forward primer as set forth in SEQ ID NO:184 and reverse primer as set forth in SEQ ID NO:185), primer pair 3 (forward primer as set forth in SEQ ID NO:186 and reverse primer as set forth in SEQ ID NO:187), primer pair 4 (forward primer as set forth in SEQ ID NO:188 and reverse primer as set forth in SEQ ID NO:189), primer pair 5 (forward primer as set forth in SEQ ID NO:190 and reverse primer as set forth in SEQ ID NO:191), primer pair 6 (forward primer as set forth in SEQ ID NO:192 and reverse primer as set forth in SEQ ID NO:193), primer pair 7 (forward primer as set forth in SEQ ID NO:194 and reverse primer as set forth in SEQ ID NO:195), primer pair 8 (forward primer as set forth in SEQ ID NO:196 and reverse primer as set forth in SEQ ID NO:197), primer pair 9 (forward primer as set forth in SEQ ID NO:198 and reverse primer as set forth in SEQ ID NO:199), primer pair 10 (forward primer as set forth in SEQ ID NO:200 and reverse primer as set forth in SEQ ID NO:201), primer pair 11 (forward primer as set forth in SEQ ID NO:202 and reverse primer as set forth in SEQ ID NO:203), primer pair 12 (forward primer as set forth in SEQ ID NO:204 and reverse primer as set forth in SEQ ID NO:205), primer pair 13 (forward primer as set forth in SEQ ID NO:206 and reverse primer as set forth in SEQ ID NO:207), primer pair 14 (forward primer as set forth in SEQ ID NO:208 and reverse primer as set forth in SEQ ID NO:209), primer pair 15 (forward primer as set forth in SEQ ID NO:210 and reverse primer as set forth in SEQ ID NO:211), primer pair 16 (forward primer as set forth in SEQ ID NO:212 and reverse primer as set forth in SEQ ID NO:213), primer pair 17 (forward primer as set forth in SEQ ID NO:214 and reverse primer as set forth in SEQ ID NO:215), primer pair 18 (forward primer as set forth in SEQ ID NO:216 and reverse primer as set forth in SEQ ID NO:217), primer pair 19 (forward primer as set forth in SEQ ID NO:218 and reverse primer as set forth in SEQ ID NO:219, primer pair 20 (forward primer as set forth in SEQ ID NO:220 and reverse primer as set forth in SEQ ID NO:221), primer pair 21 (forward primer as set forth in SEQ ID NO:222 and reverse primer as set forth in SEQ ID NO:223), primer pair 22 (forward primer as set forth in SEQ ID NO:224 and reverse primer as set forth in SEQ ID NO:225), primer pair 23 (forward primer as set forth in SEQ ID NO:226 and reverse primer as set forth in SEQ ID NO:227), primer pair 24 (forward primer as set forth in SEQ ID NO:228 and reverse primer as set forth in SEQ ID NO:229), primer pair 25 (forward primer as set forth in SEQ ID NO:230 and reverse primer as set forth in SEQ ID NO:231), primer pair 26 (forward primer as set forth in SEQ ID NO:232 and reverse primer as set forth in SEQ ID NO:233), primer pair 27 (forward primer as set forth in SEQ ID NO:234 and reverse primer as set forth in SEQ ID NO:235) and primer pair 28 (forward primer as set forth in SEQ ID NO:236 and reverse primer as set forth in SEQ ID NO:237).

In a specific example of the present invention, to identify genes of Javanese medaka differentially regulated upon exposure to BPA, Javanese medakas were exposed to 76 μg/L BPA for 48 hr in each seawater and general seawater condition. Then, mRNAs were extracted from them and cDNAs were synthesized. Finally, differentially regulated genes were investigated by using microarray. In the result, 96 genes of which expressions were changed upon exposure to Bisphenol A were found (Refer to table 5). Total 28 genes which are involved in defense mechanisms against external stress were selected and primers for them were designed and synthesized for quantitative real-time reverse transcript polymerase chain reaction (qRT-PCR: Refer to table 6). qRT-PCR was performed using synthesized primers, followed by the investigation of differentially regulated genes (Refer to table 7).

The present invention also provides a use of differentially regulated genes upon exposure to BPA for producing a microarray chip to detect exposure to BPA and diagnose environmental pollution from a specimen wherein said gene is selected from the group consisting of praline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2sex hormone-binding globulin (SEQ ID NO:152), Isocitrate dehydrogenase sex hormone-binding globulin (SEQ ID NO:153), ATP citrate lyase (ACLY), transcript variant 2sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181).

The present invention also provides a use of differentially regulated genes upon exposure to BPA for producing a kit to detect exposure to BPA and diagnose environmental pollution from a specimen wherein said genes are selected from the group consisting of proline rich 6 (SEQ ID NO: 71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2sex hormone-binding globulin (SEQ ID NO:152), Isocitrate dehydrogenase sex hormone-binding globulin (SEQ ID NO:153), ATP citrate lyase (ACLY), transcript variant 2sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181).

Therefore, for detecting stress and diagnosing conditions resulting from exposure to persistent toxic substances like BPA, said genes of which expressions are increased or decreased can be usefully exploited as biomarkers and in a microarray chip since expressions of those showed significant difference upon exposure to BPA.

### MODE FOR INVENTION

The present invention will be explained in detail with reference to the examples below. However, the following examples are offered for purpose of illustration, not limitation.

### Example 1

### Culturing Javanese Medaka and Exposure to Endocrine Disruption Chemical 17β-Estradiol (E2) or Bisphenol A (BPA)

<1-1> Culturing Javanese Medaka

Javanese medaka was cultured in natural seawater filtered through three types of filters (10, 10 and 1 μm). Water temperature was fixed at 25° C. with underwater heater, and Artemia salina nauplii were fed once a day.

<1-2> Exposure to 17β-Estradiol or BPA

To 3 L beaker with 2 L filtered seawater therein, 10 male Javanese medaka bred in culture water tank were transported each time, and acclimated for 24 hr. Five male Javanese medaka of Example <1-1> were exposed to 17β-estradiol (100 μg/L) for 24 hr and 48 hr, respectively. Additionally, five Javanese medaka of Example <1-1> were exposed to BPA (76 μg/L) for 48 hr. The concentration of exposure was set to a relatively very low level so that it was 1/100 the BPA lethal concentration 50 (LC50) of Oryzias latipes. The Javanese medaka was transported to ice water, one at a time, which momentarily stunned it, followed by beheading and evisceration and liver removal.

### Example 2

### Measuring Gene Change in Response to Exposure to 17β-Estradiol

<2-1> Isolating RNA

To the Javanese medaka livers of 17β-estradiol exposed group (24 hr & 48 hr) produced from Example <1-2>, and of control group without exposure, 1 ml of TRI Reagent solution (Molecular Research Center Inc, Cincinnati, Ohio, USA) was added, homogenized with glass homogenizer, and left at room temperature for 5 min. Next, chloroform (200 μl) was added and mixed, left at room temperature for 10 min. After centrifugation for 15 min (12,000×g, 4° C.), supernatant was obtained, to which isopropanol (500 μl) was added and left at room temperature for 5 min. After centrifugation for about 20 min (12,000×g, 4° C.), solution was removed, thus leaving precipitate. To the precipitate, 70% ethanol solution (50 μl) was added and after centrifugation for 5 min, ethanol solution was removed, and the precipitate RNA was dried. After drying, the product was dissolved in appropriate amount of DEPC (diethylpyrocarbonate)-treated water.

<2-2> Cy3 and Cy5 Labeling

The entire RNA of the distilled exposed group and non-exposed group were labeled with Cy3 and Cy5 as follows, using Agilent's Low RNA Input Linear Amplification Kit Plus (Agilent Technologies, USA). To be specific, RNA (1 μg) was mixed with dT-promoter primer and MMLV-Reverse transcriptase. After reverse transcription at 40° C. for 2 hr, T7 polymerase was added, and linear amplification was done at 40° C. for 2 hr. After the amplification process explained above, the samples of experimental and control groups were labeled with Cy3-CTP and Cy5-CTP, respectively.

<2-3> Hybridization and Scanning

Fluorescent-labeled cDNA sample was purified with Qiagen PCR purification kit, and eluted with distilled water. The purified, fluorescent-labeled cDNA sample was added to hybridization buffer (3×SSC, 0.3% SDS, 50% formamide, 20 μg Cot-1 DNA, 20 μg yeast tRNA), and concentrated with microcon YM-30. After hybridization, the hybridized compound was heated at 95° C. for 3 min for denaturalization, and the temperature of the heated, hybridized compound was decreased by centrifugation for 30 sec at 12,000 g. The produced Javanese medaka cDNA microarray was covered with coverslip, and the denaturalized, hybridized compound was pipetted. The microarray was put into GT-Hyb chamber for reaction to occur at 65° C. for 16 hr. After hybridization, the microarray was taken out of the chamber, underwent cleansing process as explained below, rotated and dried, and stored in a dark room until scanning. The Javanese medaka microarray was then scanned with Axon GenePix 4000B scanner (Axon Instrument, USA). On the GenePix Pro 6.0 program, dots from the scanned image were gridded using gridding file, and quantified to produce GPR file including analysis on cy5/Cy3 magnitudes and ratios.

<2-4> Microarray Data Analysis

Based on the GPR file obtained with the GenePix Pro Program, the following analysis was made using analysis program, i.e., GeneSpring 7.3.1 (Agilent Technologies, USA). Normalization: by LOWESS (locally weighted regression scatterplot smoothing). Reliable gene): significant genes were obtained by flagging out dots whose sums of medial values were less than the background, or dots whose standard deviations of pixel values are not significant. Significant genes: dots whose normalized ratio is at 10 times higher interval, were screened.

The result confirmed that exposure to 17β-estradiol for 24 hr (Table 1) and 48 hr (Table 2) caused increase or decrease of gene expression levels compared to the control by two or more times higher or lower, in which 52 species with 10 times or higher expression differences were identified with SEQ ID. NOs. 1 to 52 and the following nucleic acid sequences.

TABLE 1
Genes with expression differences after 24 hr of exposure to
17β-estradiol and amount of differences

| | | |
|---|---|---|
| | | SEQ |
| | Fold | ID. |
| Gene | difference | No. |

Down-regulation

| | | |
|---|---|---|
| Dimethylglycine dehydrogenase | 20.2 ± 0.01 | 1 |
| Fructose-bisphosphate aldolase B | 14.3 ± 0.02 | 2 |
| Fatty acid binding protein 10, liver basic | 12.5 ± 0.08 | 3 |
| Claudin | 10.0 ± 0.02 | 4 |
| Cytochrome P450 2P3 | 10.0 ± 0.03 | 5 |
| Aldolase B | 10.2 ± 0.05 | 6 |

Up-regulation

| | | |
|---|---|---|
| Cytochrome c-1 (cyc1) | 10.3 ± 0.03 | 7 |
| Selenoprotein M | 10.3 ± 0.03 | 8 |
| ATPase, H+ transporting, V1 subunit F (atp6v1f) | 10.4 ± 0.03 | 9 |
| Cytochrome oxidase subunit I (COI) gene | 10.7 ± 0.06 | 10 |
| Similar to ATP citrate lyase isoform 2 | 11.4 ± 0.03 | 11 |
| Ribosomal protein L13a (rp113a) | 11.7 ± 0.09 | 12 |
| Cytochrome c oxidase subunit I | 12.6 ± 0.45 | 13 |
| Pyrroline-5-carboxylate reductase 1 (pycr1) | 12.8 ± 0.23 | 14 |
| Similar to Exs-related protein (LOC100150890) | 13.0 ± 0.05 | 15 |
| Cysteine-rich with EGF-like domains 2 (creld2) | 13.2 ± 0.02 | 16 |
| Selenoprotein 15 | 13.4 ± 0.03 | 17 |
| Beta-galactoside-binding lectin | 14.9 ± 0.01 | 18 |
| hv1 gene for histone H2 variant | 14.9 ± 0.05 | 19 |
| Similar to LAG1 longevity assurance 2 | 17.1 ± 0.02 | 20 |
| Inositol oxygenase | 17.1 ± 0.06 | 21 |
| Acyl-CoA synthetase long-chain family member 1 | 17.3 ± 0.23 | 22 |
| Tetraspanin-3 (putative) | 17.9 ± 0.01 | 23 |
| Microsomal triglyceride transfer protein | 18.1 ± 0.01 | 24 |
| Amino-terminal enhancer of split (aes) | 18.1 ± 0.09 | 25 |
| non-classical MHC class I antigen (Orda-UDA) | 18.2 ± 0.03 | 26 |
| NADH-cytochrome b5 reductase 3 | 19.7 ± 0.06 | 27 |
| rRNA 2-O-methyltransferase fibrillarin (fbrl) | 20.5 ± 0.23 | 28 |
| Vitellogenin II(vit-6) | 20.8 ± 0.13 | 29 |
| Choriogenin H minor | 21.5 ± 0.03 | 30 |
| Adenylosuccinate synthetase isozyme 2 | 21.5 ± 0.18 | 31 |
| Mannose-P-dolichol utilization defect 1b (mpdu1b) | 21.6 ± 0.02 | 32 |
| RAB3 GTPase activating protein subunit 2 | 23.7 ± 0.03 | 33 |
| (non-catalytic) | | |
| Tax1 (human T-cell leukemia virus type I) binding | 25.0 ± 0.01 | 34 |
| protein 1b | | |
| 40S ribosomal protein S24-like protein | 26.8 ± 0.02 | 35 |
| Exosome complex exonuclease RRP4 | 35.2 ± 0.04 | 36 |
| Similar to Iduronate 2-sulfatase precursor (ids) | 36.8 ± 0.03 | 37 |
| Vitellogenin 1 (ol-vit1) | 53.1 ± 0.13 | 38 |

TABLE 2
Genes with expression differences after 48 hr of exposure to
17β-estradiol and amount of differences

| | | |
|---|---|---|
| | | SEQ |
| | | ID. |
| Genes | Fold difference | No. |

Up-regulation

| | | |
|---|---|---|
| Oligosaccharyltransferase complex subunit (ostc) | 100.0 ± 0.01 | 39 |
| Abhydrolase domain containing 11 | 11.1 ± 0.06 | 40 |
| Fatty acid binding protein 10, liver basic | 10.0 ± 0.04 | 3 |
| Fructose-bisphosphate aldolase B | 10.2 ± 0.01 | 2 |

Down-regulation

| | | |
|---|---|---|
| B-cell antigen receptor complex-associated | 10.0 ± 0.05 | 41 |
| protein alpha-chain precursor | | |
| Cytochrome oxidase subunit I (COI) | 10.5 ± 0.01 | 10 |
| Alpha-2,3-sialyltransferase ST3Gal I | 10.5 ± 0.07 | 42 |
| Inositol oxygenase | 11.5 ± 0.03 | 21 |
| Glutathione reductase (mitochondrial) | 11.7 ± 0.02 | 43 |
| Pyrroline-5-carboxylate reductase 1 (pycr1) | 11.8 ± 0.01 | 14 |
| Choriogenin L | 12.1 ± 0.02 | 44 |
| Similar to integral membrane protein 1 | 12.3 ± 0.02 | 45 |
| Complement control protein factor I-B | 12.5 ± 0.02 | 46 |
| Glutamate receptor, ionotropic, delta 1 (GRID1) | 12.7 ± 0.03 | 47 |
| Dolichyl-alpha-1,6-mannosyltransferase (alg12) | 12.7 ± 0.03 | 48 |
| Macrosialin precursor | 13.3 ± 0.02 | 49 |
| hv1 gene for histone H2 variant | 13.6 ± 0.06 | 19 |
| Similar to ATP citrate lyase isoform 2 | 13.7 ± 0.01 | 11 |
| Selenoprotein M | 13.9 ± 0.01 | 8 |
| 40S ribosomal protein S24-like protein | 14.6 ± 0.03 | 35 |
| Metalloreductase STEAP4 | 14.7 ± 0.02 | 50 |
| Vitellogenin II (vit-6) | 14.9 ± 0.01 | 29 |
| NADH-cytochrome b5 reductase 3 | 16.4 ± 0.02 | 27 |
| Similar to Exs-related protein (LOC100150890) | 16.7 ± 0.05 | 15 |
| RAB3 GTPase activating protein subunit | 17.2 ± 0.01 | 33 |
| 2 (non-catalytic) | | |
| Tetraspanin-3 | 17.4 ± 0.08 | 23 |
| Beta-galactoside-binding lectin | 18.9 ± 0.02 | 18 |
| Similar to LAG1 longevity assurance 2 | 19.3 ± 0.05 | 20 |
| 14-alpha demethylase (CYP51) | 19.7 ± 0.04 | 51 |
| Exosome complex exonuclease RRP4 | 21.7 ± 0.02 | 36 |
| Tax1 (human T-cell leukemia virus type I) | 23.4 ± 0.01 | 34 |
| binding protein 1b | | |
| Mannose-P-dolichol utilization defect 1b | 23.6 ± 0.01 | 32 |
| (mpdu1b) | | |
| Choriogenin H minor | 23.9 ± 0.02 | 30 |
| Adenylosuccinate synthetase isozyme 2 | 27.8 ± 0.23 | 31 |
| rRNA 2-O-methyltransferase fibrillarin (fbrl) | 31.7 ± 0.12 | 28 |
| Microsomal triglyceride transfer protein | 37.1 ± 0.08 | 24 |
| Acyl-CoA synthetase long-chain family member 1 | 40.1 ± 0.01 | 22 |
| Bromodomain containing 2 (RING3) | 45.2 ± 0.03 | 52 |
| Similar to Iduronate 2-sulfatase precursor (ids) | 49.1 ± 0.01 | 37 |
| Vitellogenin 1 (ol-vit1) | 59.1 ± 0.02 | 38 |

### Example 3

### Quantification of Expression Differences of Genes Screened as Having Expression Differences after Exposure to 17β-Estradiol

<3-1> Screening Genes Involved with Self Defense Mechanisms to External Stress

Among Javanese medaka genes having two or more times greater expression differences after exposure to 17β-estradiol in Example <2-4>, total seven genes were screened, as having high involvement with the self defense mechanisms to external stress, which are:

Apolipoprotein B, P450 1A (Cytochrome P450 1A, CYP1A), glutamate dehydrogenase 1b, glucose-6-phosphate dehydrogenase, transferrin, vitellogenin 1 and selenoprotein M.

Primers for real-time quantitative PCR (RT-PCR) for the above genes were designed and synthesized (Table 3). The expression differences of the above-listed genes were analyzed with reference to Javanese medaka livers exposed to 17β-estradiol for 48 hr.

TABLE 3
Sequences of primers of genes for RT-PCR

| Gene | Sequence of primer | SEQ. ID. NO. |
|---|---|---|
| Apolipoprotein B | F: 5′-AAGCTAATGCCGAGGTTGATC-3′ | 57 |
| | R: 5′-GGAGGCAGAGGACTTGAATG-3′ | 58 |
| Cytochrome P450 1A | F: 5′-TCAACCAGTGGCAGATAAACC-3′ | 59 |
| (CYP1A) | R: 5′-CTGGCACTTCCTCAAATCTC-3′ | 60 |
| Glutamate dehydrogenase | F: 5′-CGATGCCAGGTCCGATGAAAC-3′ | 61 |
| 1b | R: 5′-GCCCAACACAGCCAGCACACAG-3′ | 52 |
| Glucose-6-phosphate | F: 5′-AGGTGTCCCACTATCGGTTC-3′ | 63 |
| dehydrogenase | R: 5′-CTTCATCCTCCAGAGCTTGTG-3′ | 64 |
| Transferrin | F: 5′-CCTGGAATCTGACGACTACCA-3′ | 65 |
| | R: 5′-CCTGGAATCTGACGACTACC-3′ | 66 |
| Vitellogenin 1 | F: 5′-ACAAAAGGTTCCACTCTCAGC-3′ | 67 |
| | R: 5′-CCAACTTTAACCTCCATCTCC-3′ | 68 |
| Selenoprotein M | F: 5′-GGTCAAAGCTTTTGTGACTC-3′ | 69 |
| | R: 5′-TCTGGCTTCTCCTTCTTGTAG-3′ | 70 |

<3-2> cDNA Synthesis

With RNA template extracted with the method of Example <2-1>, cDNA was synthesized using AB High Capacity RNA-to-cDNA Kit (Applied Biosystems, USA). RNA in the amount of 1 μg was seeded, added with distilled water and titrated to 9 μl. 2×RT buffer (10 μl), and 20× enzyme Mix 1 μl were mixed well, centrifuged and precipitated, and allowed to react at 37° C. for 60 min. The reactant was heated at 95° C. for 5 min, after which reaction was completed. The synthesized cDNA was stored at −20° C.

<3-3> Real-Time Quantitative PCR

To the 0.5 μg synthesized cDNA (corresponding to approximately 10 ng/μl), 0.8 primer pairs of corresponding genes (10 pmol/μl), and 2 SYBR1 mixture (10 μl) were added. The final amount was titrated to 20 μl using distilled water. At temperature condition of 95° C. 10 min, 95° C. 30 sec, 60° C. 30 sec, 72° C. 30 sec, 40 cycles of PCR were conducted.

CT values of the respective samples after exposure to 17β-estradiol between control genes (β-actin) and genes of interest were compared with each other, and the relative expression differences of the genes of interest were quantified into relative values according to Relative Standard Curve (Table 4).

TABLE 4
Difference of expression level of genes of interest based on
real-time quantitative PCR

| | Duration of | |
|---|---|---|
| | exposure | |
| Gene | 24 hr | SEQ ID. NO. |
| Apolipoprotein B | 2.5 times lower | 53 |
| Cytochrome P450 1A (CYP1A) | 2.7 times lower | 5 |
| Glutamate dehydrogenase 1b | 5.2 times lower | 54 |
| Glucose-6-phosphate dehydrogenase | 2.2 times lower | 55 |
| Transferrin | 3.6 times lower | 56 |
| Vitellogenin 1 | 59.1 times higher | 37 |
| Selenoprotein M | 7.7 times higher | 8 |

### Example 4

### Measuring Changes in Genes in Response to Exposure to BPA

<4-1> Isolation of RNA

To the Javanese medaka livers of BPA exposed group (48 hr) produced from Example <1-2>, and of control group without exposure, 1 ml of TRI Reagent solution (Molecular Research Center Inc, Cincinnati, Ohio, USA) was added, homogenized with glass homogenizer, and left at room temperature for 5 min. Next, chloroform (200 μl) was added and mixed, left at room temperature for 10 min. After centrifugation for 15 min (12,000×g, 4° C.), supernatant was obtained, to which isopropanol (500 μl) was added and left at room temperature for 5 min. After centrifugation for about 20 min (12,000×g, 4° C.), solution was removed, thus leaving precipitate. To the precipitate, 70% ethanol solution (50 μl) was added and after centrifugation for 5 min, ethanol solution was removed, and the precipitate RNA was dried. After drying, the product was dissolved in appropriate amount of DEPC (diethylpyrocarbonate)-treated water.

<4-2> Cy3 and Cy5 Labeling

The entire RNA of the distilled exposed group and non-exposed group were labeled with Cy3 and Cy5 as follows, using Agilent's Low RNA Input Linear Amplification Kit Plus (Agilent Technologies, USA). To be specific, RNA (1 μg) was mixed with dT-promoter primer and MMLV-Reverse transcriptase. After reverse transcription at 40° C. for 2 hr, T7 polymerase was added, and linear amplification was done at 40° C. for 2 hr. After the amplification process explained above, the samples of experimental and control groups were labeled with Cy3-CTP and Cy5-CTP, respectively.

<4-3> Hybridization and Scanning

Fluorescent-labeled cDNA sample was purified with Qiagen PCR purification kit, and eluted with distilled water. The purified, fluorescent-labeled cDNA sample was added to hybridization buffer (3×SSC, 0.3% SDS, 50% formamide, 20 μg Cot-1 DNA, 20 μg yeast tRNA), and concentrated with microcon YM-30. After hybridization, the hybridized compound was heated at 95° C. for 3 min for denaturization, and the temperature of the heated, hybridized compound was decreased by centrifugation for 30 sec at 12,000 g. The produced Javanese medaka cDNA microarray was covered with coverslip, and the denaturized, hybridized compound was pipetted. The microarray was put into GT-Hyb chamber for reaction to occur at 65° C. for 16 hr. After hybridization, the microarray was taken out of the chamber, underwent cleansing process as explained below, rotated and dried, and stored in a dark room until scanning. The Javanese medaka microarray was then scanned with Axon GenePix 4000B scanner (Axon Instrument, USA). On the GenePix Pro 6.0 program, dots from the scanned image were gridded using gridding file, and quantified to produce GPR file including analysis on cy5/Cy3 magnitudes and ratios.

<4-4> Microarray Data Analysis

Based on the GPR file obtained with the GenePix Pro Program, the following analysis was made using analysis program, i.e., GeneSpring 7.3.1 (Agilent Technologies, USA). Normalization: by LOWESS (locally weighted regression scatterplot smoothing). Reliable gene): significant genes were obtained by flagging out dots whose sums of medial values were less than the background, or dots whose standard deviations of pixel values are not significant. Significant genes: dots whose normalized ratio is at 10 times higher interval, were screened.

The measurement of gene change in response to exposure to BPA for 48 hr caused increase or decrease of gene expression levels compared to the control by two or more times higher or lower, in which 92 species with 2 times or higher expression levels were identified with SEQ ID. NOs. 71 to 162 (Table 5).

TABLE 5
Genes with two or more times increased or decreased expression
levels after 48 hr of exposure to BPA and amount of differences

| | | |
|---|---|---|
| No. | Gene | Fold difference |

Up-regulation

| | | |
|---|---|---|
| 71 | proline rich 6 | −5.99 |
| 72 | telomerase reverse transcriptase gene | −4.31 |
| 73 | uridine phosphorylase 2 | −3.77 |
| 74 | MHC Class I Region | −3.70 |
| 75 | vitellogenin II | −3.69 |
| 76 | retinoid X receptor beta (RXRB) gene | −3.42 |
| 77 | protein tyrosine phosphatase-like, member b | −3.23 |
| 78 | TRAF-binding protein | −3.17 |
| 79 | HSPC038 protein | −3.05 |
| 80 | Glycerol-3-phosphate dehydrogenase | −3.03 |
| 81 | proteasome subunit, beta type 8 | −2.96 |
| 82 | trypsinogen | −2.88 |
| 83 | carnitine O-acetyltransferase precursor | −2.78 |
| 84 | tubulin, beta 5 | −2.56 |
| 85 | muscleblind mRNA | −2.53 |
| 86 | phosphoenolpyruvate carboxykinase | −2.51 |
| 87 | protein phosphatase 2 regulatory subunit B beta | −2.50 |
| 88 | eukaryotic peptide chain release factor GTP-binding subunit | −2.45 |
| 89 | 40S ribosomal protein S18 | −2.43 |
| 90 | trypsinogen | −2.42 |
| 91 | estrogen receptor beta 2 | −2.37 |
| 92 | bromodomain containing 2 (RING3) gene | −2.35 |
| 93 | glutathione S-transferase | −2.33 |
| 94 | cysteine dioxygenase | −2.32 |
| 95 | cyclin G2 | −2.27 |
| 96 | protein kinase C and casein kinase substrate in neurons 1 | −2.22 |
| 97 | septin 7b (sept7b), transcript variant 1 | −2.20 |
| 98 | c-type lysozyme mRNA | −2.18 |
| 99 | Uridine phosphorylase 1 | −2.16 |
| 100 | 60S acidic ribosomal protein P1 | −2.13 |
| 101 | heart-type fatty acid-binding protein | −2.12 |
| 102 | choriogenin Hminor | −2.09 |
| 103 | ribosomal protein L13 | −2.09 |
| 104 | glucose-regulated protein 94 | −2.09 |
| 105 | cathepsin F | −2.08 |
| 106 | 5,10-methylenetetrahydrofolate reductase (NADPH)-like | −2.06 |
| | mRNA | |
| 107 | mitogen-activated protein kinase 1 | −2.06 |
| 108 | Transcription initiation factor IIA gamma chain | −2.06 |
| 109 | ATPase, H+ transporting, lysosomal, V1 subunit E isoform 1 | −2.04 |
| 110 | leukocyte elastase inhibitor | −2.04 |
| 111 | Putative transmembrane 4 superfamily member protein | −2.02 |
| 112 | mitogen-activated protein kinase (p38) | −2.02 |
| 113 | DEAD-box RNA-dependent helicase p68 mRNA | −2.01 |

Down-regulation

| | | |
|---|---|---|
| 114 | Transforming growth factor-beta-induced protein ig-h3 | +2.00 |
| 115 | 40S ribosomal protein S19 | +2.00 |
| 116 | alcohol dehydrogenase Class VI (ADH8) | +2.01 |
| 117 | malate dehydrogenase | +2.01 |
| 118 | carboxyl ester lipase, tandem duplicate 2 | +2.01 |
| 119 | alpha-2-macroglobulin | +2.01 |
| 120 | TBT-binding protein | +2.02 |
| 121 | keratin 15 | +2.02 |
| 122 | complement component C9 | +2.03 |
| 123 | alpha-2-macroglobulin-2 | +2.03 |
| 124 | kelch-like ECH-associated protein 1b | +2.06 |
| 125 | hepcidin-like precursor | +2.07 |
| 126 | NADH dehydrogenase subunit 4 | +2.08 |
| 127 | cyclin Y-like 1 | +2.09 |
| 128 | complement regulatory plasma protein | +2.11 |
| 129 | protein kinase C and casein kinase substrate in neurons 2 | +2.12 |
| 130 | choriogenin L | +2.12 |
| 131 | complement factor B/C2-B | +2.12 |
| 132 | Nedd4 family interacting protein 2 | +2.13 |
| 133 | Restin (Cytoplasmic linker protein-170 alpha-2) | +2.14 |
| 134 | ornithine decarboxylase antizyme large isoform ORF1 | +2.15 |
| 135 | catalase | +2.16 |
| 136 | ring finger protein 141 | +2.16 |
| 137 | Cytochrome P450 7B1 (Oxysterol 7-alpha-hydroxylase) | +2.16 |
| 138 | hypoxanthine phosphoribosyltransferase 1 | +2.17 |
| 139 | cytochrome P450 CYP2Y3 | +2.18 |
| 140 | chymotrypsinogen 2-like protein | +2.22 |
| 141 | Iron-responsive element-binding protein 1 | +2.25 |
| 142 | calreticulin | +2.25 |
| 143 | delta 6-desaturase | +2.39 |
| 144 | kelch domain containing 10 | +2.42 |
| 145 | arginyl-tRNA synthetase | +2.52 |
| 146 | ATP-binding cassette transporter sub-family G member 2b | +2.59 |
| 147 | adult alpha-type globin | +2.59 |
| 148 | GTP cyclohydrolase 1 precursor | +2.60 |
| 149 | Thyroid hormone receptor-associated protein complex 150 kDa | +2.62 |
| | component | |
| 150 | sex hormone-binding globulin | +2.62 |
| 151 | protein phosphatase 2A regulatory subunit B′ delta isoform | +2.63 |
| 152 | cyclin B2 | +2.66 |
| 153 | Isocitrate dehydrogenase | +2.72 |
| 154 | ATP citrate lyase (ACLY), transcript variant 2 | +2.84 |
| 155 | decorin mRNA | +2.87 |
| 156 | apolipoprotein A-IV4 | +2.94 |
| 157 | Translation initiation factor eIF-2B subunit epsilon | +3.00 |
| 158 | Ribosomal protein S14, transcript variant 4 | +3.11 |
| 159 | Transmembrane protein 85 | +5.22 |
| 160 | heat shock protein 84b | +5.53 |
| 161 | warm-temperature-acclimation-related-65 kDa- | +5.88 |
| | protein (C79) | |
| 162 | glutathione S transferase Rho-class | +6.90 |

Expression changes:
−Up-regulation (decreased expression level);
+Down-regulation (increased expression level)

### Example 5

### Quantification of Expression Levels of the Genes which are Screened as Changing Expression Levels in Response to BPA Exposure

<5-1> Screening of Genes Involved with Self-Defense Mechanism Against External Stress

Total 28 out of 92 genes were screened, which include: nine (9) genes whose gene expression levels changed two or more times higher after exposure to BPA; and 19 genes whose expression levels were not increased to two times higher levels, but did exhibit self-defense mechanism against external stress, and these are:

Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO: 167), Catalase, Calcium binding protein P22 (SEQ ID NO:168), Ceruloplasmin (SEQ ID NO:169), Complement factor B/C2-B, Chitinase (SEQ ID NO:170), Choline kinase (SEQ ID NO:171), Choriogenin L, Delta-6 fatty acyl desaturase (SEQ ID NO:172), Delta 6-desaturase, Glutaminase (SEQ ID NO:173), Glutathione S-transferase, Hepcidin (SEQ ID NO:174), Leukocyte elastase inhibitor, Lipoprotein lipase (SEQ ID NO:175), N-acetyltransferase (SEQ ID NO:176), Plasminogen (SEQ ID NO:177), Retinol binding protein 4 (SEQ ID NO:178), Ring finger protein 141, TBT-binding protein, Transferrin (SEQ ID NO:179), Uridine phosphorylase 2, Vitellogenin 1 (SEQ ID NO:180), Warm temperature acclimation related 65 kDa protein (C243) (SEQ ID NO:181)

Primers for real-time quantitative PCR (RT-PCR) for the above genes were designed and synthesized, and sequences of the primers of the genes for real time quantitative PCT are listed in Table 6 below.

TABLE 6
Primer sequence of genes for RT-PCR

| Primer | Gene | | Sequence |
|---|---|---|---|
| ANAT | Arylamine N-acetyl | F | 5′-CATTCACTGTGGTGAGAGGA-3′ (SEQ ID NO: 182) |
| | transferase | R | 5′-TCCAAAGCTCACGTCTGTAA-3′ (SEQ ID NO: 183) |
| AP | Apolipoprotein E1 | F | 5′-GGATCCATACTTCTCTCAGGT-3′ (SEQ ID NO : 184) |
| | | R | 5′-GTCTGAACATGTTTCCATGAG-3′ (SEQ ID NO : 185) |
| Bsgn | Basigin | F | 5′-ATCCTCGTGACCATCATCTT-3′ (SEQ ID NO: 186) |
| | | R | 5′-AGGAACTTTGCAGAAAGCAC-3′ (SEQ ID NO: 187) |
| C8 | Complement component | F | 5′-GTGCACTATAACCCCGACTT-3′ (SEQ ID NO: 188) |
| | C8 beta | R | 5′-AGTGACCTCACAGCTCCTTC-3′ (SEQ ID NO: 189) |
| C-ASP | Clq-like adipose | F | 5′-CTGTTGACGTTGTTGCTTTG-3′ (SEQ ID NO: 190) |
| | specific protein | R | 5′-AAAAACTACTGGGGTTCGTG-3′ (SEQ ID NO: 191) |
| Cat | Catalase | F | 5′-GCGGTACAACAGCGCCGATGA-3′ (SEQ ID NO: 192) |
| | | R | 5′-GGATGGACGGCCTTCAAGTTCT-3′ (SEQ ID NO: 193) |
| CBP-P22 | Calcium binding | F | 5′-CCACAGTCAGATCACTCGAC-3′ (SEQ ID NO: 194) |
| | protein P22 | R | 5′-CAAAGTGCAGCTTGTTCATC-3′ (SEQ ID NO: 195) |
| Cer | Ceruloplasmin | F | 5′-GTCAGTTTGAAGTGGTCTCTG-3′ (SEQ ID NO: 196) |
| | | R | 5′-GAACCTATGAATCCTCCTTGT-3′ (SEQ ID NO: 197) |
| CF-B/C2 | Complement factor | F | 5′-CGTGGACATATGTGACCCTA-3′ (SEQ ID NO: 198) |
| | B/C2-B | R | 5′-TGTCCAAATCTGTTGGAATG-3′ (SEQ ID NO: 199) |
| Chi | Chitinase | F | 5′-GATGCAGGATAGAGAGAGGTT-3′ (SEQ ID NO: 200) |
| | | R | 5′-AGATCCATGGAAGTCATAGGT-3′ (SEQ ID NO: 201) |
| Chka | Choline kinase | F | 5′-CCAGAAGTACCCCTCAAAGA-3′ (SEQ ID NO: 202) |
| | | R | 5′-TTCTTCTGCTGGAAGTAGGC-3′ (SEQ ID NO: 203) |
| ChL | Choriogenin L | F | 5′-TGCAACACCAGGAAACTATG-3′ (SEQ ID NO: 204) |
| | | R | 5′-TTTTCGGCTCATACCAATAC-3′ (SEQ ID NO: 205) |
| FADSD6 | Delta-6 fatty acyl | F | 5′-TGGACAATGACTTTCCTGTG-3′ (SEQ ID NO: 206) |
| | desaturase | R | 5′-CCTGAAGATATTGGGTTTG-3′ (SEQ ID NO: 207) |
| FD6D | Delta 6-desaturase | F | 5′-TCTCGTGTCATTAGCCACTAT-3′ (SEQ ID NO: 208) |
| | | R | 5′-ATTCTTCTCTGCCTGAACTCT-3′ (SEQ ID NO: 209) |
| Gls | Glutaminase | F | 5′-TGAGTCAGGAAGTGTCATGG-3′ (SEQ ID NO: 210) |
| | | R | 5′-TTACCAAGGCGATCTAAAGG-3′ (SEQ ID NO: 211) |
| Gst | Glutathione | F | 5′-TCGACAAGATGGAGCACAAATC-3′ (SEQ ID NO: 212) |
| | S-transferase | R | 5′-CAGTTTGTTCCCCTGAGACTTGA-3′ (SEQ ID NO: 213) |
| Hep | Hepcidin | F | 5′-AACTGCTGCAAGAACTACAAG-3′ (SEQ ID NO: 214) |
| | | R | 5′-CCAACAGCCTTTATCTGTTT-3′ (SEQ ID NO: 215) |
| LE | Leukocyte elastase | F | 5′-GTTTGATGCAAAGGAAACTC-3′ (SEQ ID NO: 216) |
| | inhibitor | R | 5′-CATGAAGTTCTCGTAGGTCAG-3′ (SEQ ID NO: 217) |
| LPL | Lipoprotein lipase | F | 5′-TTCACTGTTTGGAACTCACG-3′ (SEQ ID NO: 218) |
| | | R | 5′-ACCAGATCAGCAAACTCTCC-3′ (SEQ ID NO: 219) |
| NAT13 | N-acetyltransferase | F | 5′-TCGAGATCATCGAGACAAAA-3′ (SEQ ID NO: 220) |
| | | R | 5′-GCAGAAATTGCCTTTGAAAC-3′ (SEQ ID NO: 221) |
| Plg | Plasminogen | F | 5′-TGCTGAGCAGATCTCAACAT-3′ (SEQ ID NO: 222) |
| | | R | 5′-AATGTAAGCGAAGGATTTGC-3′ (SEQ ID NO: 223) |
| RBP4 | Retinol binding protein | F | 5′-AGAACCAGGTATGCAGGTGT-3′ (SEQ ID NO: 224) |
| | 4 | R | 5′-ACCCCAGTATTTCATCCTGA-3′ (SEQ ID NO: 225) |
| RFP | Ring finger protein 141 | F | 5′-TCTGCACCAAGATCAACAAG-3′ (SEQ ID NO: 226) |
| | | R | 5′-TTTGTCGATGCATTTCTGAC-3′ (SEQ ID NO: 227) |
| TBT-bp | TBT-binding protein | F | 5′-CCATCAATGTTAGCCACAAA-3′ (SEQ ID NO: 228) |
| | | R | 5′-CGTAATGTATTCGGGTTCCT-3′ (SEQ ID NO: 229) |
| TF | Transferrin | F | 5′-TCACACAGACTCCTTCCTCTA-3′ (SEQ ID NO: 230) |
| | | R | 5′-TCAGTCACAGATCAGATCCTC-3′ (SEQ ID NO: 231) |
| Upp2 | Uridine phosphorylase 2 | F | 5′-GACATTCTGTACCACTTCAGC-3′ (SEQ ID NO: 232) |
| | | R | 5′-CCACTTTGTACATGGAGTAGC-3′ (SEQ ID NO: 233) |
| Vit1 | Vitellogenin 1 | F | 5′-ACAAAAGGTTCCACTCTCAGC-3′ (SEQ ID NO: 234) |
| | | R | 5′-CCAACTTTAACCTCCATCTCC-3′ (SEQ ID NO: 235) |
| Wap65 | Warm temperature | F | 5′-CGACAAGGGTCAAACTTTTT-3′ (SEQ ID NO: 236) |
| | acclimation related | R | 5′-TTTGGATAGCCTTCCTCAAG-3′ (SEQ ID NO: 237) |

65 kDa protein(C243)

<5-2> cDNA Synthesis

With RNA template extracted with the method of Example <2-1>, cDNA was synthesized using AB High Capacity RAN-to-cDNA Kit (Applied Biosystems, USA). RNA in the amount of 1 μg was seeded, added with distilled water and titrated to 9 μl. 2×RT buffer (10 μl), and 20× enzyme Mix 1 μl were mixed well, centrifuged and precipitated, and allowed to react at 37° C. for 60 min. The reactant was heated at 95° C. for 5 min, after which reaction was completed. The synthesized cDNA was stored at −20° C.

<5-3> Real-Time Quantitative PCR

To the 0.5 μg synthesized cDNA (corresponding to approximately 10 ng/μl), 0.8 primer pairs of corresponding genes (10 pmol/μl), and 2 SYBR1 mixture (10 μl) were added. The final amount was titrated to 20 μl using distilled water. At temperature condition of 95° C. 10 min, 95° C. 30 sec, 60° C. 30 sec, 72° C. 30 sec, 40 cycles of PCR were conducted.

CT values of the respective samples after exposure to BPA between control genes (β-actin) and genes of interest were compared with each other, and the relative expression differences of the genes of interest were quantified into relative values according to Relative Standard Curve (Table 7).

TABLE 7
Difference of expression level of genes of interest based on
real-time quantitative PCR

| Primer | Gene | Fold difference |
|---|---|---|
| ANAT | Arylamine N-acetyl transferase | −1.69 |
| AP | Apolipoprotein E1 | +2.30 |
| Bsgn | Basigin | +4.25 |
| C8 | Complement component C8 beta | −1.37 |
| C-ASP | C1q-like adipose specific protein | −2.17 |
| Cat | Catalase | −1.49 |
| CBP-P22 | Calcium binding protein P22 | +1.19 |
| Cer | Ceruloplasmin | +2.09 |
| CF-B/C2 | Complement factor B/C2-B | −2.27 |
| Chi | Chitinase | −5.56 |
| Chka | Choline kinase | +4.18 |
| ChL | Choriogenin L | +50.00 |
| FADSD6 | Delta-6 fatty acyl desaturase | −1.39 |
| FD6D | Delta 6-desaturase | +1.43 |
| Gls | Glutaminase | −1.35 |
| Gst | Glutathione S-transferase | −1.85 |
| Hep | Hepcidin | −1.14 |
| LE | Leukocyte elastase inhibitor | +1.12 |
| LPL | Lipoprotein lipase | +1.11 |
| NAT13 | N-acetyltransferase | −2.50 |
| Plg | Plasminogen | +1.25 |
| RBP4 | Retinol binding protein 4 | +1.12 |
| RFP | Ring finger protein 141 | +1.23 |
| TBT-bp | TBT-binding protein | −1.66 |
| TF | Transferrin | −1.30 |
| Upp2 | Uridine phosphorylase 2 | −1.72 |
| Vit1 | Vitellogenin 1 | −100.00 |
| Wap65 | Warm temperature acclimation (C243) | +1.07 |

### INDUSTRIAL AVAILABILITY

The Javanese medaka genes that react to exposure to one of the endocrine disruption chemicals, i.e., 17β-estradiol, or Javanese medaka that react to exposure to BPA can be advantageously used as a biosensor component to monitor and diagnose EDC contamination of hydroecosystem. Furthermore, considering ability to specify physiological changes in metabolism of living organisms based on the functions of the presented genes, use as biomarker and sensor is applicable to predict pathological phenomena that can occur, and also effective use for the detection of stress sources of hydroecosystem or health condition diagnosis is provided.

What is claimed is:

1. A DNA microarray chip for detecting exposure to Bisphenol A from a specimen, wherein said microarray chip comprises the partial oligonucleotides or their complementary equivalents from nucleic acid sequences of at least one gene selected from the group consisting of proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2 sex hormone-binding globulin (SEQ ID NO:152), Isocitrate dehydrogenase sex hormone-binding globulin (SEQ ID NO:153), ATP citrate lyase (ACLY), transcript variant 2 sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181).

2. The DNA microarray chip of claim 1 for detecting exposure to Bisphenol A from a specimen, wherein said gene is originated from Javanese medaka (Oryzias javanicus).

3. The DNA microarray chip of claim 1 for detecting exposure to Bisphenol A from a specimen, wherein mRNA expressions of proline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO:77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112) and DEAD-box RNA-dependent helicase p68 mRNA (SEQ ID NO:113) are decreased upon exposure to Bisphenol A.

4. The DNA microarray chip of claim 1 for detecting exposure to Bisphenol A from a specimen, wherein mRNA expressions of Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2 sex hormone-binding globulin (SEQ ID NO:152), Isocitrate dehydrogenase sex hormone-binding globulin (SEQ ID NO:153), ATP citrate lyase (ACLY), transcript variant 2 sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161) and glutathione S transferase Rho-class (SEQ ID NO:162) are decreased upon exposure to Bisphenol A.

5. The DNA microarray chip of claim 1 for detecting exposure to Bisphenol A from a specimen, wherein said gene selected from the group consisting of Arylamine N-acetyl transferase (SEQ ID NO:163), Apolipoprotein E1 (SEQ ID NO:164), Basigin (SEQ ID NO:165), Complement component C8 beta (SEQ ID NO:136), C1q-like adipose specific protein (SEQ ID NO:167), catalase (SEQ ID NO:135), Calcium binding protein P22 (SEQ ID NO:168), Ceruloplasmin (SEQ ID NO:169), complement factor B/C2-B (SEQ ID NO:131), Chitinase (SEQ ID NO:170), Choline kinase (SEQ ID NO:171), choriogenin L (SEQ ID NO:130), Delta-6 fatty acyl desaturase (SEQ ID NO:172), delta 6-desaturase (SEQ ID NO:143), Glutaminase (SEQ ID NO:173), glutathione S transferase Rho-class (SEQ ID NO:162), Hepcidin (SEQ ID NO:174), leukocyte elastase inhibitor (SEQ ID NO:110), Lipoprotein lipase (SEQ ID NO:175), N-acetyltransferase (SEQ ID NO:176), Plasminogen (SEQ ID NO:177), Retinol binding protein 4 (SEQ ID NO:178), ring finger protein 141 (SEQ ID NO:136), TBT-binding protein (SEQ ID NO:120), Transferrin (SEQ ID NO:179), uridine phosphorylase 2 (SEQ ID NO:73), Vitellogenin 1 (SEQ ID NO:180) and Warm temperature acclimation related 65 kDa protein (C243) (SEQ ID NO:181) is involved in defense mechanisms against external stress.

6. A method for detecting exposure to Bisphenol A from a specimen, said method comprising the steps of:
1) separating RNAs from Javanese medaka (Oryzias javanicus) of a sample of an experimental group exposed to a specimen, and from Javanese medaka (Oryzias javanicus) of a control group;
2) converting RNAs extracted from the experimental group and the control group of step 1) into cDNA and labeling them with different fluorescent materials;
3) hybridizing cDNAs labeled with different fluorescent materials of step 2) to said microarray chip of claim 1;
4) analyzing the reacted microarray chip of step 3); and
5) comparing expression levels of genes on the microarray chip between the experimental group and the control group.

7. A method for detecting exposure to Bisphenol A from a specimen, said method comprising the steps of:
1) separating RNAs from Javanese medaka (Oryzias javanicus) of a sample of an experimental group exposed to a specimen, and from Javanese medaka (Oryzias javanicus) of a control group;
2) performing real time RT-PCR with the RNAs of step 1) using at least one primer pair to amplify a gene selected from the group consisting of praline rich 6 (SEQ ID NO:71), telomerase reverse transcriptase gene (SEQ ID NO:72), uridine phosphorylase 2 (SEQ ID NO:73), MHC Class I Region (SEQ ID NO:74), vitellogenin II (SEQ ID NO:75), retinoid X receptor beta (RXRB) gene (SEQ ID NO:76), protein tyrosine phosphatase-like member b (SEQ ID NO: 77), TRAF-binding protein (SEQ ID NO:78), HSPC038 protein (SEQ ID NO:79), Glycerol-3-phosphate dehydrogenase (SEQ ID NO:80), proteasome subunit, beta type 8 (SEQ ID NO:81), trypsinogen (SEQ ID NO:82), carnitine O-acetyltransferase precursor (SEQ ID NO:83), tubulin, beta 5 (SEQ ID NO:84), muscleblind mRNA (SEQ ID NO:85), phosphoenolpyruvate carboxykinase (SEQ ID NO:86), protein phosphatase 2 regulatory subunit B beta (SEQ ID NO:87), eukaryotic peptide chain release factor GTP-binding subunit (SEQ ID NO:88), 40S ribosomal protein S18 (SEQ ID NO:89), trypsinogen (SEQ ID NO:90), estrogen receptor beta 2 (SEQ ID NO:91), bromodomain containing 2 (RING3) gene (SEQ ID NO:92), glutathione S-transferase (SEQ ID NO:93), cysteine dioxygenase (SEQ ID NO:94), cyclin G2 (SEQ ID NO:95), protein kinase C and casein kinase substrate in neurons 1 (SEQ ID NO:96), septin 7b (sept7b) transcript variant 1 (SEQ ID NO:97), c-type lysozyme mRNA (SEQ ID NO:98), Uridine phosphorylase 1 (SEQ ID NO:99), 60S acidic ribosomal protein P1 (SEQ ID NO:100), heart-type fatty acid-binding protein (SEQ ID NO:101), choriogenin H minor (SEQ ID NO:102), ribosomal protein L13 (SEQ ID NO:103), glucose-regulated protein 94 (SEQ ID NO:104), cathepsin F (SEQ ID NO:105), 5,10-methylenetetrahydrofolate reductase (NADPH)-like mRNA (SEQ ID NO:106), mitogen-activated protein kinase 1 (SEQ ID NO:107), Transcription initiation factor IIA gamma chain (SEQ ID NO:108), ATPase, H+ transporting, lysosomal, V1 subunit E isoform 1 (SEQ ID NO:109), leukocyte elastase inhibitor (SEQ ID NO:110), Putative transmembrane 4 superfamily member protein (SEQ ID NO:111), mitogen-activated protein kinase (p38) (SEQ ID NO:112), DEAD-box RNA-dependent helicase p68 mRNA (SEQ ID NO:113), Transforming growth factor-beta-induced protein ig-h3 (SEQ ID NO:114), 40S ribosomal protein S19 (SEQ ID NO:115), alcohol dehydrogenase Class VI, ADH8 (SEQ ID NO:116), malate dehydrogenase (SEQ ID NO:117), carboxyl ester lipase, tandem duplicate 2 (SEQ ID NO:118), alpha-2-macroglobulin (SEQ ID NO:119), TBT-binding protein (SEQ ID NO:120), keratin 15 (SEQ ID NO:121), complement component C9 (SEQ ID NO:122), alpha-2-macroglobulin-2 (SEQ ID NO:123), kelch-like ECH-associated protein 1b (SEQ ID NO:124), hepcidin-like precursor (SEQ ID NO:125), NADH dehydrogenase subunit 4 (SEQ ID NO:126), cyclin Y-like 1 (SEQ ID NO:127), complement regulatory plasma protein (SEQ ID NO:128), protein kinase C and casein kinase substrate in neurons 2 (SEQ ID NO:129), choriogenin L (SEQ ID NO:130), complement factor B/C2-B (SEQ ID NO:131), Nedd4 family interacting protein 2 (SEQ ID NO:132), Restin (Cytoplasmic linker protein-170 alpha-2) (SEQ ID NO:133), ornithine decarboxylase antizyme large isoform ORF1 (SEQ ID NO:134), catalase (SEQ ID NO:135), ring finger protein 141 (SEQ ID NO:136), Cytochrome P450 7B1 (Oxysterol 7-alpha-hydroxylase) (SEQ ID NO:137), hypoxanthine phosphoribosyltransferase 1 (SEQ ID NO:138), cytochrome P450 CYP2Y3 (SEQ ID NO:139), chymotrypsinogen 2-like protein (SEQ ID NO:140), Iron-responsive element-binding protein 1 (SEQ ID NO:141), calreticulin (SEQ ID NO:142), delta 6-desaturase (SEQ ID NO:143), kelch domain containing 10 (SEQ ID NO:144), arginyl-tRNA synthetase (SEQ ID NO:145), ATP-binding cassette transporter sub-family G member 2b (SEQ ID NO:146), adult alpha-type globin (SEQ ID NO:147), GTP cyclohydrolase 1 precursor (SEQ ID NO:148), Thyroid hormone receptor-associated protein complex 150 kDa component (SEQ ID NO:149), sex hormone-binding globulin (SEQ ID NO:150), protein phosphatase 2A regulatory subunit B′ delta isoform sex hormone-binding globulin (SEQ ID NO:151), cyclin B2 sex hormone-binding globulin (SEQ ID NO:152), Isocitrate dehydrogenase sex hormone-binding globulin (SEQ ID NO:153), ATP citrate lyase (ACLY), transcript variant 2 sex hormone-binding globulin (SEQ ID NO:154), decorin mRNA sex hormone-binding globulin (SEQ ID NO:155), apolipoprotein A-IV4 sex hormone-binding globulin (SEQ ID NO:156), Translation initiation factor eIF-2B subunit epsilon sex hormone-binding globulin (SEQ ID NO:157), Ribosomal protein S14, transcript variant 4 sex hormone-binding globulin (SEQ ID NO:158), Transmembrane protein 85 sex hormone-binding globulin (SEQ ID NO:159), heat shock protein 84b sex hormone-binding globulin (SEQ ID NO:160), warm-temperature-acclimation-related-65 kDa-protein (C79) (SEQ ID NO:161), glutathione S transferase Rho-class (SEQ ID NO:162), Arylamine N-acetyl transferase (SEQ ID NO:163), Apolipoprotein E1 (SEQ ID NO:164), Basigin (SEQ ID NO:165), Complement component C8 beta (SEQ ID NO:166), C1q-like adipose specific protein (SEQ ID NO:167), Calcium binding protein P22 (SEQ ID NO:168), Ceruloplasmin (SEQ ID NO:169), Chitinase (SEQ ID NO:170), Choline kinase (SEQ ID NO:171), Delta-6 fatty acyl desaturase (SEQ ID NO:172), Glutaminase (SEQ ID NO:173), Hepcidin (SEQ ID NO:174), Lipoprotein lipase (SEQ ID NO:175), N-acetyltransferase (SEQ ID NO:176), Plasminogen (SEQ ID NO:177), Retinol binding protein 4 (SEQ ID NO:178), Transferrin (SEQ ID NO:179), Vitellogenin 1 (SEQ ID NO:180) and Warm temperature acclimation related 65 kDa protein (C243) (SEQ ID NO:181); and
3) comparing gene expression levels between the experimental group and the control group as measured by real-time RT-PCR in step 2).


SEQUENCE LISTING (237 sequences)

SEQ ID NO: 1 - DNA, 812 bp, Oryzias javanicus
aacctcaagaaagtccactatgacagtatcaagctgtacgagaccctggaggaggaaactggacaggccgtaggtttccaccagcccggcagcgtccgcatcgcttcgactgcagctcgagtggatgagatgaggtaccagatgactcgcacgcactggcatgtgacgccccaatacttcataactccggagaaggtccaggaacttttccctctgctgaacatggacaaggtgttggccgggctgtacacccctggagacggtcacattgacccgtactctctgaccatggctctggcggcgggagctcgcatgtacggcgcccagatctacaaccccgctccggtgagcgctctcactccgagatctgacggcaaatgggacgtccagactcctcacggcaccattcgggctaaccgcatcgtcaacacggcaggtttttgggctcgggaagtcggacaaatgattgggtttgatcatcccaccatccctgtgcatcaccagtatgtcgtgactgcgactgtaccagaggtgaaggctctgaagaaagaactcgccgtcatcagagatctggaaggctcctactatctgcgtcaggagagagatggattgctatttgggccgtatgagaagatggagaagatggtgcttcaggactcctgggtcagggatggagtgcctccaggcttcggaaaagagctgtttgagtcggaccttgacaggataatggaacacattgaaatggccatggaaatggtcccggtgctgaagaaagctgacatcatcaatgttgtttctgggcccataacgta

SEQ ID NO: 2 - DNA, 785 bp, oryzias javanicus
tcgggaagctgtgcagctccgtgtgtcacttcacgtgtcctcgttgaaaaaacaaccctcaagatgacccatcagtatccatacctgtctccagagaagaagaaggagctctctgacattgctcagagaatcgttgcttcaggaaagggaatcctggctgcagatgaatcaacaggaaccatggctaaacgcctgcagaagatcaaggtggagaacacagaggagaaccgccgttgcttccgtgacattcttttctccagcgatccgtcgatcgccaacaacattggtggggtcattttcttccacgagaccctctaccagaaggcagacaacggcaagcttttccctcaggtcatcaaggagaaaggcattgttgttggtatcaaggtggacaaaggcacagctgaacttggcggaactaatggagagacaaccactcagggtcttgatggtctgtcggagcgatgcgcccagtacaagaaagacggatgcgactttgctaagtggaggtgtgtgctgaagatctccgatggctgcccgtcagctctcgccattgcagagaacgccaacgtcctcgctagatacgccagtatctgccaacagaacggtctggtgccaattgtggagcctgagatcctgcctgatggtgaccacgacctgcagaagtgccagtatgtgacagaaaaggtcctggctgccacctacaaggctctttcagatcaccacgtgtacctggagggcactctgctgaagcccaacatggtgactgctggacatgcctgctc

SEQ ID NO: 3 - DNA, 776 bp, oryzias javanicus
acagctgtctcagcctctagccgccgtggtgaaggaaaaactcaaaatcttttaagaaaaatggacttcaacggatcatggcaggtgtactcccaggagaactacgaggcgttcctcagggctttggaactgtctgaagatatcattaagctggccaaggatgtcaagccagttactgagatcaaacagacaggcaacgacttcgtcatcacctccaagactcctggaagaaccgtgaccaactccttcaccatcggcaaggaggctgaaatcaccaccatggatggcaagaagctaaagtgtgttgtcaacattgaaggtggcaaactggtctgcaaaactggcaaattttgccatgtgcaggagatcaagggaggagaaatggttgagactatgaccgtcggttcaacaacactcatcaggaaaagcaaaaagatgtgaatttaacaacaaagaacaactttttaataaaaaatgtgcttaaaaaataaaaaaaaaaaaaaaaaaaaaacagctccgtgtgtcacttcacgtgtcctcgttgaaaaaacaaccctcaagatgacccatcagtatccatacctgtctccagagaagaagaaggagctctctgacattgctcagagaatcgttgcttcaggaaagggaatcctggctgcagatgaatcaacaggaaccatggctaaacgcctgcagaagatcaaggtggagaacacagaggagaaccgccgttgcttccgtgacattcttttctccagcgatccgtcgatcgccaa

SEQ ID NO: 4 - DNA, 759 bp, oryzias javanicus
aaatttaacaaaaaaacctccctttattgtgcttaaagaactttacaaaactgtacaaaaaacacacaaaaataacatttgaataattttgactttgttcatacttctggtcatttcagctcagatcatcaaatgattatgttttttttcagcgtacactatcaattaaaaatgaaaacattcattcaggaaaagtagggggctttgtacaatgtttacaaaaggacgtccttgtattgaagtcaaacacgtgcagttgtgcttggttctttccagttgtgtcatggaccaaaagttccaatttaaacccacaacatgcatcagaaacagcaacaaaaaaagaaaaacgtaggaaaatgtccccgctcttgtgtccttcagacgtaagcgtttcgtccataagtgctggcgactacacttctggtcggtgctgcaccggagtaaaccgttcctctggcttgatacgggatattgtatttttcctctctcgccactttgcaggaacacgtgaggcagactcccccggcgatggcgagcactccggagcaccagccgatgtaaagcccttctccgatttcataccttatccccggatagaaagggtcaaagaaatcctgcgtgatgttgaaggcataccaggacaccgacaccatggtgcacaaaccttgaagtataaagaagactccagcagtgccagcaatcctccctttgaggagatagttttccccccctgctttggagcactggatccctatgagg

SEQ ID NO: 5 - DNA, 773 bp, oryzias javanicus
gttgcctcattcaagagaattcaagtgactagagtcaatccctttgctctagtgagtgactcagcaaccaaagaccgtacaataagtcaggcagataaagctgctctttagtgagagttcagagagaatttgctcactcttggctaacaatggaggcaataactgctgttctgggattggaatggatcgacaccaggagcattttgatatttctgtttgtctttctgttgctgagtgattatttagtcaacaggcgaccaaaaaactttcctcctggcccccattctcttcctttcattggagatcttcatcacatcagcccttccaggcttcacctgcagctcacagagtttgcagaaaaatatggaaatgttttcagccttcatctctttggacaaagagcagtgatcctgaatggacacaagcaggtgaaagaggccttgatccaaagaggggacgactttgtggatcgtcccagcgtcccaatgtttgagcagttttattcaaacaaaggcattgtggggtcaagtggttatccatggaagcaacagaggagatttgctcttcatacactaagaaactttggtttaggcaagaagacaatggagaaatatatccaggaggagtgccactatctctcagaagcttttgcagagtacaagggtaagccctttaatgcccaggcactgataaacaatgcagtgtcgaatatcatttgttgccttgtttttgggggaacggtatgagtattctgataaacagtaccagcaaat

SEQ ID NO: 6 - DNA, 875 bp, oryzias javanicus
gtcacttcacgtgtcctcgttgaaaaaacaaccctcaagatgacccatcagtatccatacctgtctccagagaagaagaaggagctctctgacattgctcagagaatcgttgcttcaggaaagggaatcctggctgcagatgaatcaacaggaaccatggctaaacgcctgcagaagatcaaggtggagaacacagaggagaaccgccgttgcttccgtgacattcttttctccagcgatccgtcgatcgccaacaacattggtggggtcattttcttccacgagaccctctaccagaaggcagacaacggcaagcttttccctcaggtcatcaaggagaaaggcattgttgttggtatcaaggtggacaaaggcacagctgaacttggcggaactaatggagagacaaccactcagggtcttgatggtctgtcggagcgatgcgcccagtacaagaaagacggatgcgactttgctaagtggaggtgtgtgctgaagatctccgatggctgcccgtcagctctcgccattgcagagaacgccaacgtcctcgctagatacgccagtatctgccaacagaacggtctggtgccaattgtggagcctgagatcctgcctgatggtgaccacgacctgcagaagtgccagtatgtgacagaaaaggtcctggctgccacctacaaggctctttcagatcaccacgtgtacctggagggcactctgctgaagcccaacatggtgactgctggacatgcctgctctaagaaatacacccctcaagaggttgccatggctacggtgactgctctgaggcgcactgttcccgcctcagtccctggcatctgcttcctgtctggaggccagagtgaggagga

SEQ ID NO: 7 - DNA, 699 bp, oryzias javanicus
ttcaaagatggctgcgttgcgggttgtggcggtgtcggggagcgggagagccctcctcgccgctccgaaggctcttcgtgccccccaggcgaacatgtcctttgccagcctaccaattaagaagaaggttgccttgacgactttgggtgtgatcacagctggtggagcgagcctggccttgatgctgcaccagtctgtgaaagcctcggacttggagctccatccaccaaactacccctggagccacgccggacctctgtctgcgctggaccacgccagcatccgtcgtgggtaccaggtgtacaagcaggtgtgctcggcctgccacagcatggagtacttggccttcagaaacctggtcggggtgtctcacacggaggaggagatgaaggccatagctgaggaggttgaagtggtggatggacctgatgagaacggcgagatgttcacccgtccaggaaaactgtcggactatttcccaaagccctacgccaaccccgaggcagcacgtgtagccaacaacggggcgctccctccggacctcagctacatcgttaacgccagacacggtggcgaggactatgtgttcagcctcctcacgggctactgcgacccgcctgcaggggtcaccgtccgagaaggactctactacaacccctacttccctggccaggctataggcatggccccgcccatcta

SEQ ID NO: 8 - DNA, 858 bp, oryzias javanicus
gtcaccttcgtgcatctgcgggtcccggttcgacaggagaaccgatcctgacccgggaggagaatgtggctgatcgcgttggccgccgttcttcagtgcgcctccggttatgatgtcgacgtgaagaagctggagggaatggcgcgcgcgcgggtggagacctgtggtggatgacagctcaacaggctcagagaggtcaaagcttttgtgactcaggatattccactttaccataacttggtgatgaagcacatccctggagccgatcctgagcttgtcctcctcagtcactattatgacgaacttgataggatcgctctttctcacatgacccgcactgagatcaatgagctcttgggggaactggggttctacaagaaggagaagccagaagatgaggtgccagaggagttccgcttctcccccgccaaagacagcccgtttaaagacgagccccagcatacatcaaccacctctgagccgaaagcagaagctgagcacactgacctataacctgcaagaccaagaccatcatagctgctgacagaaatgtttgtaattccacaaaagaaatatctgggaaatcttacatatatgtgatggcctgttaaaggccctctctgaataagcacctatttttcattccacttttttggggtcgtatcaggttattttgtgggtgattttctactgtccaaacagaaattgcaagtgtgtctgtgaaatgatttttatttcccctttttgtgcgttttccttcctgctgtggaactgaatgaagcttcaggctgaaaatcttctgactggcctgaggctgatgctttttgttctgctgggtaggacggatgcacacactct

SEQ ID NO: 9 - DNA, 474 bp, oryzias javanicus
agaaccgaaccgagcagtgatggcgggccgcgggaagctgatcgccgtgatcggggacgaggacacgtgcacgggcttcctgctgggcgggatcggagagctgaacaagaaccggaagccgaacttcttggtggtggagaaagacacgagcgtgacggagatcgaggagacgttcaagagcttcctggcgaggaacgacatcggcatcatcctgatcaaccagttcatcgcagagatgatccggcacgccatcgacgcgcacgtgcagtccatcccggcggttctggagatcccgtccaaggagcacccctacgacgcgtccaaggactccatcctgcgccgcgccaagggcatgttctctgcagaggacttccgatgagccgccacgccgccacacggcgtgctgccgtcctccaggcgggtccggcccgcttccacttcctgtttgttcttgttgtgaagtcttgaatgatt

SEQ ID NO: 10 - DNA, 238 bp, oryzias javanicus
ttctcgactaaacattaaaacatcggctccctctatttatgcttccgtgcctgagcaggaataatatgcactgatctgaccctgccagtccgagctgacctgagtctccccggttcccgcctgggatactaccagattgataatggaaatgttaccacacgtgcctttgtcttagttttctttccagctatgcctcttatcattggaggttttggaaactgactaatcacttaaatga

SEQ ID NO: 11 - DNA, 707 bp, oryzias javanicus
ccggatccaccttcatggaccacgtgctgcgctaccaggacactccagggattaagatgatcgtggtgttgggagagattggagggaccgacgagtaccaaatctgccagggcatcaaagagggccggatcaccaaaccggtggtgtgctggtgtattggaacctgcgccaccatgtttgcttcagaggttcagttcggtcatgcaggagcctgcgccaaccaggcctccgaaaccgcggtggccaagaaccaggccctgagggacgccggcgcttacgtgcccaggagcttcgacgagctgggagacgtcatcagaaccgtttatgatgaactggtggccgacggtactattgttccagctcaggaggttcctcctcccacagtacccatggattactcctgggctcgggagttgggtctgattcgtaaacccgcctccttcatgacgagcatctgcgacgagcgaggacaggagctcatctacgctggcatgggcatcacggaggtcttcaaagaggagatggggataggaggagttctgggtctgctctggttccagcgcagactaccacgctacgcctgccagttcatcgagatgtgtctgatggtgacggcggaccacgggcccgccgtctccggagctcacaacaccatcgtctgtgctcgcgctggcaaagacctgatctccagcctcacctctggcttg

SEQ ID NO: 12 - DNA, 634 bp, oryzias javanicus
  misc_feature (634)..(634): n is a, c, g, or t
tgctgatagatggcaggggccatctattgggccggctggccgccattgtggctaaacaagttcttcttgggcacaaagtggttattgtgcgatgtgaaggcatcaacatttctggcaacttctatcgaaacaagctgaagtacctggctttcctgcgcaagaggatgaacaccaacccctctcgtgggccataccatttcagagctcccagcaggatcttttggaggacagttagaggcatgttgccccacaaaaccaagagaggccaggctgctctggagaggctgaaggtgtttgatggcatcccacccccctatgacaagagaaagcgcatggttgtgccagctgctctcaaaattgtgcgtctgaagccctctcgcaagtttgccctcctcgggcgtctcgcacacgaggtcggctggaagtaccaggccatcacagccactctggaggagaagagaaaagagaaggccaagctccactatgtcaagaaaaagactttggttaagctaaccaagcaggcggagaagaacgtggagggcaagatcgcaaaatacacagatgttctgaaacagttcggtgttcttgtctaagctggtagattctggcaattaaagatgagtaaatgtttatn

SEQ ID NO: 13 - DNA, 152 bp, oryzias javanicus
tttatcatctgggaagcattcgcggccaaacgggaagtgctttccgtggagcttacggccacaaatgttgagtgattacacggctgccctcccccttaccacacatttgaggagcccgcttttgttcagattcagcaacctcactactagac

SEQ ID NO: 14 - DNA, 861 bp, oryzias javanicus
ccacgttggcggcagcggagttctgattcggttcgcttctgttcgaaggacgaacatctgcagctccacgttagcttcggctggatcatcagaggtgtaaggggccacgtcacctggaaggatgagcgtggggttcatcggagcgggacagctggcgcatgcgctggtgaagggcttcactgcagcaggtgtgattgctgctcagaggatcacagccagcgctccggacacggacctgcccaccgttagcagcctcaggaaaatgggggtgagtctgaccaccagcaacaaagagacggtcagcaagagcgacgtgctgttcctggctgtcaaacctcacatcatcccctttgttctggacgagatcggcccggacatcgaggaccgccacctcatagtctcgtgtgctgctggagtcaccatcagctccatagagaagaagctgcttcagcaccgcccggctcctcgagtcatgaggtgcatgaccaacacccctgtggtggtgagagagggcgccactgtgtacgccacaggaacacatgcagaggtggaggacgggcggctgctggagcagctgatggccagcgttggtttctgcacggaggtggaggaggacctgatcgacgccgtcaccggtctgagcggcagcggccccgcctacgcattcacggctcttgacgctctggcggacgggggcgtgaagatgggtctgcccaggaggctcgccgtcagacttggagctcaggccttactgggggcagcgaagatgctactggagtcggagcagcacccgggccagttaaaggacaacgtttgctcaccagggggcgccaccatccacgccctgcacttcctggagag

SEQ ID NO: 15 - DNA, 359 bp, oryzias javanicus
ggactcttccctagctcacgcgtttttgttcttgatgatcaccgccgaccttctacaggacccggaatggagagagcttgtattctgatgatttcctccctgatccttattgtggctccagaattccaaccaaacctccccatcaccaacaccagctgtggggtatccaaatactgcattacggaaccagctaactgtgaccctgagggcaaccgcacctgtatgttcggatcgctgaacgtcctgtcccaaactccttcacatggcatcaacctccacaaccagctgagtggaaactccagcggatacatcgctctggggctcactccatcatgggggaacacctcgctgtacatctg

SEQ ID NO: 16 - DNA, 817 bp, oryzias javanicus
tacaaccagcatgtaaatattacttccccgtgctttgcaagggtaccgctatttataatttaatgttagcagcggcttatttagatgtgtcccagcgctagaacgctttagcagcagcggggagtttgcttgtcagtggcttcattctgtgttgatgagtgagtgttgactgagatgctgtccgtcggtccacagcagctccgactgttgtcctgtgtcgctatccttttatttttccacgccatcgtcgccaaaaaggacttaaaaagcgcctgcagcacctgtcgacaaattactgataacttcaacaagggttttgacagaacagcaaagcagaactttggaggaggcaacacggcttgggaagagaggaagctttccaagtatgagacaagtgagattcgcctggtggagattctggaggagctgtgtgagagcagcagcttcgattgtaatcgaatggtagaagaaaacgaagagctttttgagacctggtggttcaagaggaaaacgaaaaacccggatctacacaagtggttctgcattgagactcttaaagtgtgctgcccaaagggaacatttggggctgattgtaccgcctgtgtcgggggatcggagaagccctgccacggacggggagtgtgcgacggcgacgggacacgcagcgggaacgggaagtgcagctgcgaccacggttacgagggagacctctgcttggactgcacgaacgggttcttcagcgccgagcggaacgacaccttctctttgtgctcagaatgcgatgcagcttgcaagacctgcgacggtccaaccagtc

SEQ ID NO: 17 - DNA, 840 bp, oryzias javanicus
tttcctgttacgaagcagactgcaagatgtcggctgaagtgaatctgctgtggcttatttcactactccaaacgctcctggcctgtggagccgagctgtcctctgaggcctgcagggatctgggcttctccagcaacctcctgtgcagctcctgcgacctgctcggggagttcagcctcaccaagctgcagccagactgcggacaatgctgccagcaagaggctcagatggacggacgcaagctctacgcaggcgccatcctggaggtgtgtggatgaaaattggggaggttccctcaagtccaagcttttgtcaggagcgagaagcccaagatcttcaagggtctgcagatcaagtacgtgagaggctccgatcctgtcctcaagctcctggacgataacgggaacattgctgaggagctcagcatcctcaagtggaacaccgacagcgtggaggagtttctgagtgagaagttggaacgcatataaacaagaagaaactttgatttttttcccctagcttgttttcattaatcacaaaaactgttttctttctgatcagctgtttgtgctcaaggtggaacaatgtctctgaaatctctgctccttatgaattcctccatgtcattatgtttgtgtgcaaatttgaccaaatgacgttgagggccctgacgcgtcctctcagaccattttcagttctgttttctatgtaacgctcatcagtttgacagaggagaagctctgtcctggttcctctgcaaacgcaggcctcagcagagacacacgtgtgtgaagacttcacagcaaccctgatggaatgtgaactctgatgttactttgt

SEQ ID NO: 18 - DNA, 523 bp, oryzias javanicus
gacaacatcgcattgcacttcaacccacgctttgactcgggatcagacgtcaacaccattgtctgcaactccaagtctgggggaagctggggtgaagagcagagggagggacacttcccctttgcacgtggagaggaatcaaagttttacatcaacttcaccatggagcaattttacatcaaacttcccgatgggaggatgatggactttcccaaccggctgggagaggtcaagtacgactactttgaagtcaagggggatgcagttttccatggagtcaagatcaagtagaggctaagcccctccttaaccctggcctaaagcttgttctgtccattgctggaatagttttacattaatgtgcacttggctgaaagcacagagttttgctatgctggaaaaaaaaaataataataacatcagtggagatgtaaaccggagccgctaagagatgatggcctgatggtgaagctacagcatatttcacttgttcatttggtgtctgagtacggtaataaaagct

SEQ ID NO: 19 - DNA, 866 bp, oryzias javanicus
tgaaaaggattgtagtttattaaaaaaaataatgtctttcttccattgatgttattgatgacaaatgatgagttgtagtgtttagttttatccacctgatccttccccgctgaatgacaatgtgtctaactgaaacctgtaataacatgcttgttgctaacctacaattatcagagccttggggtaagaatagttgattcagtgaaacaaactttatcattgatagtgtaatacggttcactactgatatttaagagaatcctcattgacacaaagtagcatataaaaaagtcaaattttctagattgtgtattccaaaaatgttggcttttaaaggaaaaaaaatgcatcagtttacagctgttgcatgcttttagtcttctcaacaaacatctgttctcatcattattccctacttttgcttttgagaatatattttatatcttttggctaccattctgcaacaaacattttgattttttttatacatataattttcaggtttttttgccttgcaaatattagatatgggaataagcagaaataaaaagtatttcaaaaatatgtacaaacaagcatataattaaaaaattgaatgtattctctgtaatgaaacaatgtgtttggttttgattaaaaacagtaaaaaaaaaaagccaaacgttttacacaaaaaagcaaatgctagtaattactacaatttttgagttcaatatatttgtatttatttatttttgtaaatgagaacagtttgtttgaagacaaaaaaatagtatgcaaaaaaagcattagtgacaacaagtgaaaacacctaaaaacgttcaatttgaagctagacatacttccatgaaagcgttttgagtttt

SEQ ID NO: 20 - DNA, 832 bp, oryzias javanicus
taaaaaaaatttaaaaagtggggaatgcttccagagagttcttaagacgtgcagcagtcaggagggtgtgatctccagagaggacgagtgggaggttgggatctcaggctttctcaccagagtgactccaccaaggcaacaagaggaaacacttacgatctagagttccattcgttagaagagcgctatatggactgtcttcagtctcctctgttgaaacttgtcaagacaacaccgcctccagtttgttttcatatccagtcaacttcccttctgactacaaatctggtggacagctaaaagtgccatgtttcagactgtcggcgagtggttgtggtgggagcgtttgtggctgcccggaaacgtctcctggtcagatctagaggacagcaatgaccgtgtctacgctaaggcctctcatctttacgccaccgtgccctgtgccctctgtctgctgctggtcagatacacctttgaaaggtactttgccacaccgctggctaatgcttgggggatcaaggacaaagtacgtcttactgctgagcagaatcccatcctggaaaagtatttctgcagtcaagcacgaaatccctcacagtttgatgtgaggtctctgagtaagaagaccggctggtcagagaggagagttgaaatctggttcaggagaaggaggaaccaggagcggccggggcttcggaaaaggttctgcgaggccagctggagatgtggtttttatctttttgcattttgcggtggcatcgtggccctttatgataaaccgtggctttataatctcaaagaagtttgggcagaatttcctaaacagtccatg

SEQ ID NO: 21 - DNA, 871 bp, oryzias javanicus
gtcactgcagctgctgggaaccgcatcgatcatgaggatcgtcagcataggccctgacccgtcgttggcatatcggccaaatttgatgcctgaagcaaaagagaaggaagactacagaaactttaagactggaagtctgttcgaccgtgtctataacacgtacaaactgatgcacacacaccagacgctggattttgtcaaacaaaagcactctgaatggagctgctgcagccgcactcagatgagcatgatggaagccatcatgtctctggaccagcttgttgacgagtcggatccggacgtggacttcccaaactctttccacgcctttcagacggctgaaggcatccggcaggcccacccggacaaagactggttccagctggtgggtctgatccatgatgttgggaagatcttagctctgtgggatgaaccacagtgggctgtggtgggcgacaccttccctgtgggctgcgagtttcaaaactcaatcgtgttcagatccaactccttccaggagaatccagatgacaaaaactctgcatacaggactaaatatggaatctatgaaccaaactgtggacttgacaacatctacatgtcctggggccacgatgaatatctttacagagttctaaagaaaaatgaatgttctgtcccagaagaggggatgtacatgatccgctaccattctttctacccctggcattcacatggagactacatgcatctgtgcaatgataaggacctacagatgatgccgtgggtccaagagttcaataaatttgacttgtacacaaaaaccaccgatctgccagacgtggataacctgaagccatactaccagtcactgatcgacaagtactgtcct

SEQ ID NO: 22 - DNA, 839 bp, oryzias javanicus
cggcccaaggccctgccgccgatgtatgacctcctcaggcagtcagttgaagtggagggggaagattttgcgcggcgctctgtttttacagacgacggcagtttcctgactcacatttacgacgacgccataacgatgtacgagtccttcctcagaggggttaaagtctcccaaaacggtccgtgtttgggctccaggaaaccgaagcagccgtatgagtggatttcctatggcgaggtcaaacaaagaagtgagaacctgggctcagcttttctccataaaggacactcaaagagtgtggatccccacattggtatcttctctcagaacaggcctgagtggaccatgacggagttggcgtgtcacacatactcactggtgtcggtgcctctgtacgacacgttgggaacggaggccatctcttacatcataagcaaagcgtccatctccaccgtggtgtgtgacgtgaaggataaggtcaacctcatcctggactgtttcaaggactcaaagcattcgttaaaaaccatagttttgatggaggagccggatgaaagcctggtggagagggcgcgggaagctgaaatccagatcctaagtctgcaggagatggaggctttggggagggccaaccatcagcggccaattcccccacagcccgaagacatggcagtgatttgcttcaccagcggaacaacaggaaacccaaaaggagccatgctgactcatggaaatatcatgtcgaactgctcgtcttttgttaaactgacagagcttagctgtcccatagatcacacagacgtccacatttcctatctgcctctggctcatatgtttga

SEQ ID NO: 23 - DNA, 818 bp, oryzias javanicus
aatagtgagattatcaactttttttttaatttatgataaacacaacaaagcagcaaactaaaagtagaacctcacataatacagttaactgaccataaaatgacgtgtgtttattcagctataagagtaagccaaagtaagagatcacagtctgaggtcattcgatgaaatacagtcaaagtctaaagaatatcttcaaggtgcagcagaaagttacaataaaagtcaaaatatacgatttttttccactttttcctttatgtcaggacaacttcccaaggatttagcagaaataagatgccgcatcaccttcagaatattatataaatttcattttacgtgcattcctaaagtataaccataactgggctgctttactacaccaaaagtgactgatgaaaatctcaaacaaaagaaatttaaactaacagaaaagacggaatgtccacgtttcagaaaaaaaaaaacaagacacatatggaagccagctctgatacaatctccttttagattatggcaaaactttgattaattgatatttctgtacaataatgaggaagctgaatatggatgttttggcagaaggggtttcaggcgtacaggggctggtagccattcctccggctgttgcttttacaggtgatcacacacacgctcagcattccaaagaactttatgatagcaaaaccaagaaccaccagcatggcataagtcaaagcatcctgaaggaagcgctccagtttgacctcacaaccctctgtgtaaataaggttcagttgatccagactgccggtgcactgtgtcacattggacctgca

SEQ ID NO: 24 - DNA, 786 bp, oryzias javanicus
gaaacaggagaggctccgactcgctttgcccctctctcctctctttttcggccttgttggagcccgtcagccactcactctcgtggtttgaacgatccggctgcagggagagctcacaatgtttgctgtggttctgatcctgctttgtgctgcctcctctgtctcagcatcagctaaaagtgctgctgctggacctcggctacacaacaaccagctgtacaaattcagctacaccaccgaggtccagctggacaaggccaaggggtcaaaagagggcggagctggctacaaaatctcctctgaggtcaacatcaacctggtctggagggatccaagcagcaaggatgaccagcttattcaactagctatctcaaatgtgaagattgagcacgtgtctccaagatcagagaagaaaaacatcctccaaggatccacggttgaaagtgtcttgggtaaaggcaaactgacagatctgaccaaacctttcctggtgcatctaaaaaatggaaagacaaaagcgttttactcttactggacagaacctgcagttatcaagaacctaaaaagaggactggctagcttgttgcagtttcagctcctaactgggaaggttgtggagaatgacatttctggaagatgcactgtccagtaccaggcagctaaagatcaaataacaagaaccaagatcctggacacatgcaagacttcagaatctggatttaccacacatagtaaggtgttgggtgtgggcaggaagtccagctctgttacagttttcactcttga

SEQ ID NO: 25 - DNA, 763 bp, oryzias javanicus
agatcagacattcttttgaacagcgttttcttttctttccactcatcgccttggtcgaaagagcgacctttccgtaaaaccaagacgacaatctctgcttcccgccgcttcgacgttagctaacgttattttaaaggatgacagagtacaaagtgtcagcagcgtctcgcataattttataatatctggggacttgttagcttagctagctagctagcttggcgtctcgattagatcgtaaacgcgcagcgaaaccaaacactctgctgacatgatgtttcctcaatcaaggcactcggcatcctctcagtccagtcagcccctgaagttcaccacttctgactcgtgcgatcgcatcaaggatgagttccagttccttcaagcacagtatcacagtttgaagttggagtgtgacaaactggcgtctgagaagtctgagatgcagcgtcattatatcatgtattatgaaatgtcttacgggctgaacattgaaatgcacaagcaggctgaaatagtgaagaggctgaatgggatctgtgctcaggtgctgccttacctgtcacaggagcatcagcagcaagtcatgggcgccattgagagagccaagcaggtcactcctcctgagatgaactccatcatacggcaacagctgcaggttcagcacctgtcccagctccagggcctggcgctgcccgtgaccccgctgcccctgggcctcacccctccctccctgcccgccgtctcctccagctccggcctgctg

SEQ ID NO: 26 - DNA, 830 bp, oryzias javanicus
cgttatctatccagagctggtgaaggacctgcagtgctacggctacacagcaactataactaaatgctcctggttcccggccaccaacgcttcagatgtgggatttttctggtatgcacattagtgaattgttctcacacacatttgctctgtgttcagcccagatactccaggagtgtcaaactcaatcacagagggggcaaaaatccaaaacaccctttaggtcacagactgaacaggataaactacttttttaaaactttaaaactgtaactttttaacataattatgaactagaaatatatcattacctgtgataatgctagtgttaatgctgggagatgaatttggcctctgaagatgctgaaattgatagctgaaaacactgaggctgatatctgaaaaagctgaagttgatagccagctaaaatattagctaaatgccaaattatccttaaaaaactaaactaaaaccttatcttagccaaaacaggtaatctgttgctgaatattttgctaaatttcaaaatagcctaaaaaaaagcctaaattagtcaaaacagctagaatgtaaatattagcttaactttcaaacagcctaaaaatcttattaaatgccaaaatagtataacaagcttgtagaatgtcaatttctaaaaccttaaaactgtacatttttaacataataatgaatgggaagaatattattccagaataaatcaactttaaacctaaataacttaatattttactcttcataaaaatatattttgtccaaattatacaagttagaaatcagcgcaagataacatcaggacattaataacagt

SEQ ID NO: 27 - DNA, 690 bp, oryzias javanicus
gaagatgagtcagtacctggagagcctcaagatcaacgaaaccatcgacttcagaggcccgagcggactcctggtctacaaaggcagaggtgtttttgacgtccagcctgataaaaagtctgcagcggtgaagaagacggccaaacatgtgggcatgatcgctggaggaacaggaatcactccgatgctgcagatcatcacagccatcatgaaggatcctcaggacagcacggtgtgtcacctgctgttcgcaaaccagacggagaaggacatcctgctgcgaccggagctggaggacatccaggtcaagcatccggaccgcttcaagctgtggttcaccgtggacagagcgcctctggactgggagtacagtcagggcttcatcagtgaagaaatggtccgggagcacctgccccctcccggcgaggacaccctcgtcttgatgtgcggcccccctcccatgatccagttcgcctgcaacccaaacctggacaaagtgggtcactccgagagccgcaggttcaccttctgaagacctgctcagaagcactacgttagctggaatcgcaccatttctgtatttgtttgccgtgcaaagacgacgctgaaggaccaagacgagggaagtgccttaaacacagcaaagctttcgtacctttaaacccgtttgtatgaacgtttcactgcaac

SEQ ID NO: 28 - DNA, 781 bp, oryzias javanicus
cagcttccacgcgcggttggagtcgtgaagagacaccgtggtccacttcgcacgattttggagcgaaaatgaagccaggattcagtccccgtggagacagaggaggaagaggtggaggacgaggtggaggaagaggagggtttggtgaccgtggaggaagaggcgggttcggtgatcgtggaggaagaggcgggttcagaggtggacgaggcggaggtttcaggtcccctgacggtggagggtttcggggacgcgggggcggcagaggaaccccaagaggaagaggagggagaggaggccgtggaggatttggaggagggaagaaggtggtgatagagccacacagacatgaaggggtgtttatctgcagagggaaggaggatgccctggtcaccaaaaacatggtggtgggagagtcggtgtacggagagaagaggatgagcgtggaggagggggatactaagatcgagtacagagcctggaatccattccgctccaagctagcagctgcaattcttggaggagttgaccagattcacatcaagccagggtcaaaggtcatgtaccttggagctgcatcaggcaccacggtgtcccacgtctctgacatcgtcgggccggacggactggtctacgcagtggaattctcccaccgatctggccgtgacctcctcaatgttgcaaagaaacgcaccaacatcatccccatcattgaagacgcccgtcatccgcacaagtaccgcatgctggttggcatggtggacgtgatctttgctgatgt

SEQ ID NO: 29 - DNA, 150 bp, oryzias javanicus
ctgcattcgctcacagctcgcagtcatgagggggctggttcttgctctctctctggcccttgtggccgccaaccaactgagcttcaccccagaatttagccctgggaaaacctatgagtacaaatatgaagcacatcttctgggtggcct

SEQ ID NO: 30 - DNA, 827 bp, oryzias javanicus
gcagttgctctgcatggccatctggcaaatggtcagggtgacaaggtaggtcatcagcctcagtatccttcatatcctcagaagcctcagaatcctcagcaacctcagcagcctcagtatcctcagcaaccccagcagcctcagtatcctcagcaacctcagcagcctcagtatcctcagcaacctcagcagcctcagtatccccagaagcctcagcaacctcagcagcctcagtatcctcagcaacctcagcagcctcagtatccccagaatcctcagcaacctcagtatccccagaatcctcagcaacctcagtatccccagaatcctaagcagcctcagaatcctcaggtgccaacatcaacatttcacacttgtgaagtggatgagcaccataagataccatgtggctcctcaaacatcacctcctccgagtgtgatgctataaactgctgctttgatggatacagctgctactacggcaaatatgtgactcttcagtgcaccaaggatggtcagtttatcatagtaattgccaaagatgctacactaccccacattgacttggagacagtcagtttcctgggagcaggtgctgagtgtcatcctgcaggagccacctcggcttttgctatttatcaatttccagtcacttcctgtggcacaatcatgagagatgagcctggtgttattgtctatgagaaccggatgtcctccttttatgaagttgctattggacctcgtggagccattaccagggacagtcattttgacctgtctgttcagtgcagatatattggtacttcggttgaggccttagtcgttga

SEQ ID NO: 31 - DNA, 798 bp, oryzias javanicus
ttgttttattcatagaagcaagatgaggaagtgtgtgaccggcgttaaggttggagaaatgaagatggactctgtacatcatcttgtccttaaatttacacaaaccttcagcccactctgctgttttcagaaaactactttttcattcactcccgtaattttatttttttttttacaatctaaacaagagatgtttctggtttaattcaacaataaaacagtttttcttcacaaagttacaaaagctggaaatgttgtgggtcagcgtaaagtcctttcacaaaaaggcttcaggtctgttcatcagcggctctagagtataaaacagaacaatgccagactaaaaccaactgtgctttacaataatgggaattattggcccttttctacctggtttaaaatcaacaccctcttaaaaatggaatcgacgtgaagaagtttcggctaaatctgcagaacaccagcaaacgggacagaaccagcagctatgggtccacaggtggtgctggtgaagctcctaggctccatccaggcgttttcaacttgattggagaggagtcgccgccccttaaaacagtttgatcatgctttctctggacttgccgactccgacccacttcactggcacttgcaggaagtcctcgatgaaccgaatgtaattttgtgcctgtggaggaagatcctcgaagctcctggccgtctccgtgttgcagcaccagccgggcagcgtctcgtagtccaccagcacccgcgtcaacacgtccatgtttgctggaaaacttggcagaggctgcccgt

SEQ ID NO: 32 - DNA, 443 bp, oryzias javanicus
cccggacaaacgggttttatttcatccttcagtctaaacaaacccggaagggaaacaacaactttccccaaatctagggaatctttacccaatcaaagggttaaatcgatcggccctttgattttccccctaaacccaccaaatcaaaaccagaaaactggacccaacttcttccccagcagggggaaaagaaatgggaggagttcaaatctgcgtatccccccacacaaagtccaaagtcggatcaagtcaaaaagtccggggggccctaccccctcttcttcttcttcttctgttgggtttgcgggccccggttccagaaaaacaaaacctgcggggcaataatgcctttgcagggggagaaaataacttaagtcaaggccatcagcgtgtccccggtttcctggatggaggtgaaaatccaagccagggatccggcaaac

SEQ ID NO: 33 - DNA, 831 bp, oryzias javanicus
agacatcctgcgacgacacctggtctgtgaactgtacaaccaaggcctggacctgcgcgctgaagagctgacgttagaggtggaggataaagacgtgttgggctcccagctgttggttctgacgggtcagagactcagttattctctgctgcacagccagagccagactcatgctgccatggagctgctggcacgcctccccccaaccctctgcacatggctgaaggctatggaccccagtgagctgcggtgccccctagtgcctctgtcccagaccagccgcttggtgagccgcctgattgaaatcctgccagaaaaccacgcccagtactctctggccttacacctcctggaggctgtggacgtcctcatcacggaggaggactgaagagccgggtcctggtctgttatacagtcatcctcatcatgtctctgtcatagaagctgcttttaaaggttttacttcatcaggttgagtgcatgaatcagaatctggttgaatctgtgggttaagcagagcgttgtcatagaagacttggtcctctcgtgccacatgagtttagtttctctgctctctgttagtcaacatgtaaataaagaagagccttatttttacctcgcctgtccctctatgcccaactgctttcactaagaaataaagaggctatgagcaaaaaaaaaaaaaaaaaaaactcgaaactagactgctgctagatcttttttttatttttaaaacaaataatcaatttgcacagcagcgactagcactttatagagaagattattcttagttttatcgcttcgctacctctgttttgcccccgctgcttc

SEQ ID NO: 34 - DNA, 801 bp, oryzias javanicus
gattttaaaatggccatgtggaactataaaccattgtggaaaagccccttttcggaaatgacgtaatgttgttattattcccggaagaagacccagggaaagcactgaactgcgtcaacaatagtttcttcaaattatttggatttccattgggcttgagaatgccagcaagcaactgctgaatgatttatggcactgattctggatggaagcgctctaagcaacgacatggacagatccaactttgctcacgtggtcttccaaaatgttgggaagagttatctgccccacgctgctctggagtgtcactacaccctgacacagtttatcaaaccacatccaaaggactgggttggtatatttaaggttggttggagcacggcgagggacgaatgcaactcggacatccttgtggtcaccaccaaagccggcttccttgagcaaaaggtggaagaagcacagagagagaaagatgagctcctcaaaaatatggctgtcctggagcaggagaaagagcagctgcgagcagagatggaggccctgcagaaggagtgtgagcaacagaaggagacctgcacccggctgagaagagagaaccaagaagtgcagcattcttcccaagccctgctggaggagaaagaggaggccaagaggagactggaggaggccacagctaaaattgtacagttggaggaagatctcattggggtcacacagaaaggccttcaaaaggaaacagagttagacaaccttaaagatagaatgaagaaactcacaacagagaaggaggtccttgaaagt

SEQ ID NO: 35 - DNA, 877 bp, oryzias javanicus
tcgtttagcagtcatgggaaagtgtcgtggtctgcgtactgccaggaaactgcgcaatcaccgccgtgagcaaaaatggcatgacaaacagtacaagaaggcccatctgggcaccgctctaaaggccaaccccttcggaggagcttctcacgccaagggaattgtcctggagaaagttggtgtggaagctaagcagcccaactctgccatcaggaagtgtgtgagagttcagctcatcaagaacggaaagaagatcacagccttcgtcccaaatgatggttgcctcaacttcatcgaggaaaacgacgaggttcttgtggctggttttgggcgaaaaggtcacgctgtgggagacattcctggagttcgtttcaaggtggtcaaagtggccaacgtgtctctgctggcgctctacaagggcaaaaaggaaagacccaggtcataaactgttgagaccacagataaaataaaggtttcaattacaaaaaaaaaaaaatccatctcaagccagattgtatccagcgaagattcttcttctgatgaaatttcctcagaaattctggaggaagatgacaaacacaaggttccatccttgtctaagagatactgtgccaagtaccgtgccattggactcaaagcctgcttcaaggtcgccactgaaaatgctgctttcatccgtgactctgctctgtatagaatggttggacgccacaatgcttctttctctatcaaaccaattgaaggtgaagctgcagttgagagactggagatggaagttaaagttggagctaaagcagcagagaagatcattaagcaaatcaacatgaatgataaagcgggaacagtgctgtccaagctcaacaaaatcctgacttca

SEQ ID NO: 36 - DNA, 814 bp, oryzias javanicus
gcaagtcgagtgaagttatatgctagcataatatacggtttggttggcatttattataatttgatcggataatcactcgttattattaataataaaatattaaaatactatttacctcaattgatgctttttatattgccaatctgacttgaaatagtctgaaagaagtcttaagcgaatcagaagaggtgttgcgggaagcagcatggctcttgacatgagattaccgactgtgaggaaaactgtctctctatcaacttcggctttcgatcgaaaggatctggttgtgccaggagacataatcacttcagacacaggttttatgagggggcatggcacctacgttgatgaagaaaagctgacagcttcagtcgctggagaggtgcagagggtggataaactcatctgtgtcagaccactgaagaccaggttagtcatgaaaaatataaaataatttgtagactaacccattcagattggactggcactacttttgtaaatattctataacatttgtgcataattttagatttaatggtgaggttggagatgttgtggttggcagaattactgaggtgagttattttaattgttcatcaaggatgcaagataaaatatatattttttattttttgctaataatttttttaacctgtttgctttggatggttataaaaaggtgcaacagaaacgctggaaggtggaaactaattccagactggactccgtcctgttgctctcatcagtaaacttgcctggaggagagctgagaaggagatcagcagaagatgagctgaccatgagagaatatctt

SEQ ID NO: 37 - DNA, 749 bp, oryzias javanicus
aaaatgagtctggcccctgaccccgacgttcccaaactccttccagctgtggcctacaacccctggacagatgttagaaagagagaggacgtccaaaagctcaacgttagcttcccgtttggacccattcctacagactttcagttgggtatccgtcagcactactacgctgccgtgtcgtacatggacgctcaggtggggaggctgctcagtgctctggatgggatggggctaactgatagcaccatggttgttttcacatctgatcacggttggtctctaggcgagcacggcgaatgggccaaatactcaaacttcgacgtagcgacccgcgtgcctcttatcgcgttcgttcccggcgtgaccgccgaccataacgcaccaacggaaccatcatttcccttcatagacgtcttcagcggatcagagcgccgcttccaaaatgataaagttgtgaggaacatggtggagctgctggacgtctttccaaccgtttccgctttggccggcctcagagggcccaaacactgtccggaagtctccctgcaggtggatttatgtgtggaggggaagagtctggccgccagctttaaacacaaagagaagaagaagaacgcggagagaatcgccttcagtcagtacccgcgtcccgccgacacgccgcaggagaattctgaccttcctgatctgaaagacattaaggtgatgggttactcgctgcgcacctgggactacaggtacactctgtg

SEQ ID NO: 38 - DNA, 839 bp, oryzias javanicus
ctctatgtctggccctagtggcttgttgatcctgtgatgcatggtttcaccggcatggggccgaaggaccagggagaacaagttgaggatatccctaaagcacttgaaccagagtggagtactcccatcaagtttgaatacaacaatggcattgtaggaaaaatctatactcccgatgggttctcagatatagtgctgaacttctacaaagccgttctgaacattcttcatcttaacatcaaaaagacacagaacatttatgatcttcaggaggatggaattcagggcgtgtgcaagacattctacactgtaaatgaagatgttaaggctgaccgcatcctcctgactaaaactagggacatgaacaactgtcagacaaggatctatagggacatggggttggcatacactgagaaatgtgacaagtgccaacaggaatcaaagaatctaaggggttcaacatcgtacagatatgccttgaaaccagttcctagtggtatcatgatcctggaggcagatgtaaatgagttaatccagttctcgccattctctgagggttacggagccgttcaaacagagaccagtatacttgttttttttctttttacaaaacttaaagacaaaccttggtcttcgaggagattcaggattcccccattgcaccaaaatctgctgaatatcatcactgtggatctcttaagtatgagttctctgatgagtttgagtggacaccctttcagtttgtcaaggtcactgataaacaggcccagattgaggagcttctgaatcatctaactacccacaatactgagaaggtcaatgacaatgcacctctgaag

SEQ ID NO: 39 - DNA, 815 bp, oryzias javanicus
aagtatggagactttattcagtgttccattctctgtgctggagtgtccgaacattaaactgaagaaaccgtcatggctgcacatgccgtcggctatgaccgtgtacgcggtcgtgatcgtgtcctactttctcatcacaggaggaatcatctatgatgttattgtagaaccaccaagtgtgggttcaatgacagatgaacatggacaccagcggccagtcgcctttttggcgtacagagtaaatgggcagtacattatggaaggactggcctccagtttcctcttcacaatgggaggtttgggatttataatcctggatcgctccaacgcgcctaacattcccaaactgaaccgcttcctgttgctcttcattgggttcgtcagtgtcctgctcagcttcttcatggccagagtgttcatgcgcatgaagctgccaggataccttatgggttaagaccacaaacggaagcaataatgaagttggcgtcccaaatgcagtttgagtcactgatgtaacgaattaccaaatcagaaattatggaaggaccgggatcggaagcaccgaccctgttctggaaactggattgaacgacagcgaccgcgctgacttcccatctgctattgttgttgaattgaatatatatctcatttgtgttaccacttgtcttatattttgaccaaactgaagagttgactgcaaatgaaaaatgcagaataaggaaatgaaagttaaactgagtcaaagaagtcttcatccagattcatattttgcagtaaagtttaagtcatcagattttgtatggaacgaattgagt

SEQ ID NO: 40 - DNA, 780 bp, oryzias javanicus
gctctctagcgtcgacttcaggcgctctgaatgctcgctcgctgcttgtcagctgtcatagtggaggttccgaaacgcacatgtttgtggaccgatcctcatcggaacatatgctgtcatgagcgctctgtgtcgccttgttcagagggggttgctgggctgtcggccgccgctccgtttgttccccggacagcaggatgtttgcgggatggctctgacggtccggagcgccagttcatccagtcctgtcaacctgacgtacgacgttttcgatggaaagggagagagcactcccctggtgtttctacacggcctttttggcagtaaatctaacttccattctattgccaagtccttagtgcagcgcaccggccgaaaggtgttgactgtcgatgcccgtaaccacggcaacagccctcacagccccgtactgacctatgaggccatggccagtgatttgaaacacctccttagccaactgcgaatcgagaagtgcgttctcatcggccacagcatgggagggaagacggccatgacgacggctctgacacaacccagtctggtggagcgtttggtggtcgtggacatcagtccggcccagtccgccacgcgcaccaacttccgacaatacatccaggccatgcaggagatgaagatctccactgacatcccgcgctccacagccagacggatggccgaggaccagctgcgcagattagtcaaggagcgctctgtgcgtcagttcctgctgactaacctggtggagcagaatggacatta

SEQ ID NO: 41 - DNA, 841 bp, oryzias javanicus
gcacgaggaaaaggtggaaaagtttattacgtctgagcatgtgaatgtgactgatcatcagagagaaaataacatcatttgcagcatcctgacctttagttcagtgaagctgaccgacactgggatgtaccattgccgactgaacgggacctccacgctcacacacggcacctacatgcaggtctatgagccgatggaaaaaacgatcaacctcagcgaaagcaccaaaaacgtcatcctgatgactgaaggaatcctcctgttcctgtgtgtgacggcgccatctttcctctttctgtttaagaaaaaacatgaaaataaacgggcaatcaagaaagaaaaaatagaagaggaaaacatttaccaagggctagatttggatgactgttgtgcagcatatgaccagattgaacgccctcagggacatggtccttacgaggatgtgggcacaactgtcaaagagggggaagaaatccagctggaaaaaccctgaaggaaagtagagggaaaaaagccaccaggctctttggaaattacacagtagctatattgagcttttgccaggagctttgaaaatgcctgttttgagatcagtataactcgccattataattttaaagaaaagttgttgtgcatttaaacaaatatatacgtgtttgcaagaaatttcatcacaaagttgaattttttttttttttaaagctgaaaatgagttgtcaaaatgtaaataatgaggttcccttagcaaaaagtagtgcacttaaattttatttttttaagtatactttagtacaagtaatagaaagtttactaggaattatactaactgcatacttcttca

SEQ ID NO: 42 - DNA, 836 bp, oryzias javanicus
agcagacaaactggctctcactgaggacgtccacggagaagtgagggcagcttgttctcagaggagcttcttcgttcctgtcgtccgctctcctgaccatgggggggttgtctgctgagctcccaaactgattcttgtctggataggatgtctccactcactcagaacaaattctgggctttttttctcctgtgctgcatcctcacctgcacgactctgctgttcggctacaccttcgccgaccccccctttcatttcctcaagtatgcttctcggatttctggcagctttttctccaaagacatctgtgcatgtgatacgtgcgtggcagatgtggggggcgacgcttggttggctgaccgcttcaatcagtccatactccctctgatgaccccggacaacagcgccctctctgatgacacctacaggtggtggcagtggctgcagagggaggggaacccagcaaactacaccagggtggtggaggagctgttccaacttatcccagatgaggggggtttcgtggacgccggcccgcagcgctgcaggacgtgctctgtggtggggaactccgggaacctgaaggggtccagttatgggaagctgatcgacaccagtgacctggtcatacggatgaacaaggcgcccaccaaaggctttgagaaggacgtgggagccagaaccacaccatgtcatgtacccagaaagtgcgatagatctgagcaacaccaccagtctagtgctggtccccttcaagactctggacctgcagtggatcattagtgccctgaccaccggcactatcacgggaacatacatgcctgtgaaggcctgga

SEQ ID NO: 43 - DNA, 851 bp, oryzias javanicus
cagcatggcctcggattccgacgcgacccgcttcgatttcctggtgatcggcggcgggtctggaggtctggccggtgctcggcgcgcggcggagctcggggccaacaccgccgtgatcgagagtcacaaactcggaggtacctgcgttaacgttggctgtgttcctaaaaaggttatgtggaatgcagctgtccatgcagagtatctacacgaccactgtgactacggctttgagactgcaaatgttcgtttcagctgggaagctcttaaagccaaaagggatgcatatatagcccatctaaatcgcatttatcgcaacaatctagacaaggctaaagtcaccttcattcaaggctatgccaggttcacagacgacgctgagcccaccgtggaggtcaacgggaaaaaatacacggcgcctcacatcctcatctccactggagggcagccttctgttctgagtgacgaggaagttccaggggcgagcctgggcatcaatagtgatggcttttttgaactggagaccctcccaaagcgcagcgtgattgtgggcgctggttatattgccgtggaaatggctggcattctttgcaccctgggctccaaaacctccctcattatccggcagacgggagttttgagaaacttcgacgccctcataagcgcaaactgcaccaaagaaatccaaaacaacgggatcgatttgtggaagaactcccaggtgaagtcagtgtgcaaaacggacaaaggcctggaggtgaccatcgtcaccaaagacccggacaagagcaacgacgagaagatcagcgtcatcgaggaggtggactgccttctgtgggccatcgggaggc

SEQ ID NO: 44 - DNA, 496 bp, oryzias javanicus
aaacatctttcttgctttaaggaagttgttgggactttaaattttttttgacaagtttgaacatataaagttgaggaaaggctaaatagagcagtataacccaatgcaagggtttattttgatgatcattgcaacaccaggaaactatgttacacatatgcctattaaacccccctttcttcaagggggatgggacccaaagggacatccccttgccattccaaaacttcaaatttggatgctgcccaaactttccttgaagggtctgaaggttttccaaatccgtgaccccccccactaataattgaggggccaggggatactgctgtattggtatgagccgaaaatcccctcaactcacagggggcgcaaactggatctgcaccactgacctccctccatccattgacataaaagcaacctttgtgttcaccgtcaaggggaaaagcaacaaatgtccccttcccctggcaggggatgtaaatcattccgctgt

SEQ ID NO: 45 - DNA, 833 bp, oryzias javanicus
cctcccagatttaccagctttttatttagagtattttgtggcaaaaaaaaataaaacacagtctccaaaagcatataattgaacagtgctttccccaacacatttagcaaattatgcctcctggtgagagagaaaaagacatccattaggcatttgtctaccccaaaagataaaaaaaccaaccaaaaaaaaggtattcttgtagactgcatggaaagtgttcagtgccatgcactcaaaggttattgtctccgccgtctgaagattttatgtccttgaaagacctcggttgtcaagatctttcaccttgtatatcctgaccagccagtgctctgttgtgtaggcctcctccaacacatccagttcaaagtctttgttaccgatctcagcgttccttactctgtcgtagccaggtggtcttttgccctctgtgtagacctgaccaaagcgatagtagcacatcttgtacatcaggcagttgagaagcacaggtgagccttcacgatctactctgaactctccagtgggagtgtagtagtcatgttctttgatatgtttgcccgtctctgtgcttcctccaatgcggaccatccacagaaacttgttgatatcatctgaagagtagccagtgagtcctccaaagatgacgagaacataactgacatccagctccctcataatctcataggctctctcctctgttgatgccatggcctgtccaactctagagatatgcgtgttgttccatgtgttgttgtccactagaatagttcgattcgccatggctgttatctgatagccgtaatcccaccatgacatgactttggcatcct

SEQ ID NO: 46 - DNA, 760 bp, oryzias javanicus
gaatgtggcctgcagggaggagcataacaatccagaactgaaaagcaggggtgcagcgtcggccttcaccgtggactactcccagatccgcggcggacaggagcagctcccgcgacggtgcgtcagggtccgctgtcagggcttcgagtcatctctggccgagtgtgagatctacaacaaggtggacatcggtgacggggcggtggctgcagctaactgcagcgactccaaacctgaggactgcggcttcacctgtgcgaacgacaagtgcgtgtctctgaagcagacgtgcgacggcgtggacgattgcggcgaccgcagcgatgagatgtgctgcaaacaatgcaggaatggagcgttccgctgcaacacgggggtgtgcgtgcacgccgacgccgtcggcgacaaccagctggactgtctggacggtgaagacgagtcgtccaaacagctgaattccttcgctctcagaaccgccaaacgaccagaagaaccttcagactacgtctctcgcaaaaacgagactgtaacctccagacgtctgctggagtccaaactgagctgtgggattcccaacatggacctggtggatgataccgaggagcaggcgcgtgggcagagaagcagaaagaaaagggtggtgggggggctaccggccaggcctacccagatccagtggcaggtggctctgcaggagtacaacaagatcgactgtggaggagcttatgtgggcgggtgctgggtcatcactgccgctcac

SEQ ID NO: 47 - DNA, 763 bp, oryzias javanicus
aaataaagtagatgaattccacatcctttatttttttaaagccagagatctgagcaagagatagaaaggaaagattcatctttatttaaaaaaaacgagagattcaggaggaggagactgtgatgagattcagaaagttggagacacctataggatctgctttaaagtgaaagaagatcaagaaagggttttgcaaaagaggtcacacatcatctgactgtgagcagcagcagctccttacaggcctctgatcagccatcgacacgccacgggcagataatcgccaaagcaaacacaaaaaaacttgagaagatttttcaaattccaaaagctaacaaaagcagctcgataaaatccactgtagtctgaggttagattttgatacagaggaggcagtggtgaaaggggatgttgtgaaggaatcaggaggagcttttgggactgcagctgagaaatgggagcaccaggtagacaagatcttcatcaatctcactgagaaatatacttgctatcatgtgtttgaacctaaactggtcaagaagctacagcaggatctcagcaatgtggctgatgatataaaagtgtactctcaggcaggttatgtcgtggtggtgggagaaactgttgttgtgaaggagatggtttcagttttagagaagggtcttacaaccaccatggagattccaatcacagagaaacacctcaaactgatagaagaagagttcagaagagaaatgcaagctgattctccagaagttaaaat

SEQ ID NO: 48 - DNA, 845 bp, oryzias javanicus
ttttattcacaaagttgtctttttttttaatagagaagtccatcggaggcattacctgtagaaaaaattactttatttcgtcaacgctgggggttagtctttctctccatcagaacggtcttcttctctagtcgtacgctggcaggggggaagcgagctaggtcgacggcgaggttgtggtaaccttggatgaaggctatcggctgatgagtgttccggagcagctggattctgttgtcgtcggcctccatcaggagatgggagaacatttggatttcagggcttgtggggctcaagtcttctcttttgttatatctccagattctgttttgctgcaagaagcgtgatactccagtttcagctgcgtaagtgtcaatatgaagagaaacatctgctgtggggggcagtattctgtgcagctccagcatccctcgtcctcctgggtaattgtggtgagaaatgtagagacagacgccggagtagagagcgttgagcagcagctgagcgaccacaactgctgaacccagtttgtacatccaagacttccgatagttgttcaaaataaaagagcagccgcgcgccgccaccaaactgaacaccgggaaggtgtagatgatgaagcgcagctccttgtgaggcagcagcgagtagatggcgatgaagcccacggcggggagcagcagcggcctcatccgcgcatcaagaaggccgaggggcacgaagagcagcgtacagccgaggccacgcggcacggcggagtaaaagtaccacagaaaaggggaggttccccagttggagcttttgttaagtatggtgttgtaccacaacacctgcccctcgggcca

SEQ ID NO: 49 - DNA, 850 bp, oryzias javanicus
agccatcgaagactgacactgagggtgggtgcatgaagacagacgctgaattgaaacttaaattccaagaaggcttcatatatttccactttaacaagagtggtgacatggtccacgtcgatactctgtcttttaccctgaaataccccttagtgaaaggaggtatggaatacagtgcctccagcaagtccacgacattgttctctgcacgcagcggacactcttattcctgcaggagtcagtctgtgaacatgggtgacggactgtatctggatgtgactcaggctcaaatgcaggcctttgatttacaaaatggcaattttggtaaacctgacgcctgccctgcggaccagccggactaccgtgttgccatcgcggtgggggtgaccctgctggtactcatcgtggtggtggtgatcgtgtacttgctgggtcgcagaaagaggaccgacggctaccagtctctttaagcagggggggcggagctttacgaagatgtctactctattaatgtggctttttgtggctttactgtcaccctgagaggacatttgtattaacctacttattagagccctggttttttcccgtccctggttcccatccacagaaagtgcttttaaacaaaagtattgagcgagctactgagcaccttactccacacagtccagtctgataatgcaggctgattgtcatttgatctaaaaaatgtaattttattgtagacacaaaggcagtttcaagttacttctgccatgaatttattaatggatctcttatacaaataaacaaccacgggaagatagcaaaaaaaaatgaatacaacaggttaaaaacgcatagccactga

SEQ ID NO: 50 - DNA, 855 bp, oryzias javanicus
gttttaaagaaccaacagcgctcaagttttggacaatttggagtcatatttcaacacagctccttttctttctccgaatctgcatcttgctgagagttgtcggccagctgagcagcatgtcagaggaggtgaaacctgatagcgtgttgctctgctctgtcaacacagcagccgtgccacgacacgagctgttgtgcatctttgggacgggggacttggggcgctctctgggtcagcgcctgctccagtgtggatacaaagtggtctacggcagccgcagacccggcagttgtggccccttgcctccaggaactcaggcgctgaatcatgaggcagcagctcaacatgcccatttgatctttatttgtgttcaaagagaacactacgactttatggagccgcttgcgccacaacttaaaggaaaggtgttggtggatgttagcaataatctcaagaaaaatatgtacacagactctaatgcagagtatctgcagaggctgatccctggagctcatgtggtgaaagcttttaacacattgtctgcctgggccctccagaacggacccgcagatgctaacagacaggtgtacctgtgtggaagcaatgctgaggccaagcaagttgtggcagaaattgctaccaaaatgggtttcagtgttctggatagaggatctctgtctgcagccagggaactggaagacttccccttgcagctgttcccagggtggaggctgcccatcaggctgaccattgggctcactgcctcgtttttcttctatttgctccctagagatgtcgtgtactcctacattgtacaacaaaaggacatgtcctttccggatcatggtgtctttg

SEQ ID NO: 51 - DNA, 733 bp, oryzias javanicus
ttcttcctggccagagacggctcgctgcaggagcgctgctacgccgagcagaaggcggtgtgtggagaagagcttcctcctctggactttgatcagctgaaggacctcagcttgctggagcgctgtctgaaggagacgctgaggctccgcccacctatcatgaccatgatgagaatggcccgcacgcctcagacggcggcggggttcaccatcccggcgggacaccaggtgtgcgtctcgccgaccgtcaaccaccgcctgcgggacgcctgggccaagaggacggagttcgaccccgaccgctatctccacgacaacccggccgccggggagaagttcgcctacgtgccgtttggagccggccgccatcgctgcatcggagagaacttcgcctacgtccagatcaaaaccatctggtccacgctgctgcgcacctaccagttccagctggtggacgggtacttcccctccatcaactacagcaccatgatccacacgccgcacaaccccgtcatccggtaccggaggaggacggcgtgagggggaggagccgggacggcgtgaggggaggagccgggacggcgtgagggggagggaccgggacggcgtgaagagggggaggagccgggacggcgtgaggggtccaggaagatccatgaatgactgaatgaaagcgatgtttttctgtttccatgtttccaaaaatgaagaaaaataactcatttttatattg

SEQ ID NO: 52 - DNA, 863 bp, oryzias javanicus
gaaggcggacatgttggaaggaaacgtgtttgttaaaagtatcgaggtggaagacgagatgattattggaagaaaggtttcgcattgttttggatttacacatcgatacttattaataatttaatttaaatgcttttttaaaaaacctaaaatctcgttattttccttttttgattttttttaaatcaaggatttgttaacatgatcaaaggatgactggttttggttgtataaatttagtcgtccatattgtttttcttcatttcttagtgacattgtggggaatatgtaaccttggtgtgggggatgggcctgttggttaggtaacttttgttaagcacgatttaagttaattcaatttaaatagattgttttaattattttctgttaattcacgagtattgtgatctaaaatcttaatgtaattcaccagtgcttatagagttattttttggggggttaaatttcccccctttcactgtttttaatatgtattaattagtctaaattactcaccttcttgttttatgtttcgttttttttgtttgaattttaaatacaggtcgaggtcactttgtctaaagtgctgtgtcattctgttcgtgtagcacagcgacgtgaccctctcaatgttgtcgtctgcttgatgtaaatctgcgtttcagtacgttctgttctttcctttcttttacgtttacagatattcgtataggttgtataatttattttttaagggttgatttttggcctttacaggccttctttgtctgatctaaagattttgttttgacacgttttagatgttggtgaatgtagttagtgtatcattttgtaaatgattttttttaatttatttcaattga

SEQ ID NO: 53 - DNA, 639 bp, oryzias javanicus
ccatggataaaatttatgctcaactgaagtacactggaaagaatgaagcaaatctttttggcattgaaaccacagggaagcacagtgttcagaccaacatcgacctcacaccatccgcaggactaaaagctaatgccgaggttgatctgcatcagtcaagcactctgagtgaatttatttacctcgagaagctttctgctgatattacactgtccaaccaaaagatctctttcgaaaacaagcttgacagtcctctttactcctcaaacaatgaagtggctttggagggcagttttcctgcctacacggcagcattcaagtcctctgcctccactcccatcgttctcttggaatatgatgtagatgcttctacttctgtcaactttgaaaatgaccttttggaagtacttaacaagtttgcttttacacacccaaaactgagagtgtcttccaactatttaataaccaaagctttcagaggacaaattgaagatgacgacagctgctctcatcatgatttaaatgttggcatcaccagtcctttgttcactgaagtcaaccttcgttatggcgtacagaaatgtgccatcagtgcatctgtatctactacctctggttccctcatcggccttcagctca

SEQ ID NO: 54 - DNA, 797 bp, oryzias javanicus
ggacacggtgaggagctgctgtgtctctcattctaccccctccgtgtagtttagttcttttctatttttttctcgtttacatcggtagcttcttgttttagtcatgtatcggtattttggggaactgttgagccgctctgcgggcagtgccctggcctcgggatccgtggactccgctctccccgtgtcgtcgtctctgatgcgggtccgtcgctatgccgatgcggccaacgagcccgacgaccccaacttcttcaggatggtcgagggcttctttgaccgcggcgccaccatcgtcgaggacaaactggtggaggacctgaagaccagggaaagccccgagcagaagagacatcgcgtgcggggcatcctgcgcatcatcaagccatgcaaccacgtcctgagcgtgtcgttcccgatcaagagggacaacggagagtgggaggtggtggaggggtaccgcgcccaacacagccagcacagaacaccctgcaaagggggtatccgctacagtacagatgtgtctgtggatgaggtgaaagccttggcctctctgatgacctacaagtgtgctgttgttgatgtgccatttggtggagccaaagctggagttaaaatcaaccccaagaattattccgacaatgagctggaaaaaatcaccagaagattcactattgagctggccaagaagggtttcatcggacctggcatcgacgtccctgcaccggacatgagcaccggcgagagggagatgtcctggatcgccgacacatacgccaacaccattgcacacaccg

SEQ ID NO: 55 - DNA, 573 bp, oryzias javanicus
ggtacctgaaggtcggcctctggtctttgttttggggcgtccaggtgtgcgtctgcctctcgcgggtcttcatcgcggctcacttcccgcaccaggttgtcgctggtgtcatcacaggcatgattgtggccgaggccttcaaccgaacccagtggatctacagcgccagcatgaagaaatacttctacaccacgctcttcctcacctccttcgcggttggcttctacctcgtgctcaaagctctgggcgtggacctgctgtggaccctggacaaagcccagaagtggtgcgtcaggcccgagtgggtccacctggacaccacgcccttcgccagtctgctgcgcaacatgggcacgctgttcggcttgggtctgggcctgcactcgccgctctacacagaaaccaaaaagagcaacagcgccgcggtcaaagcagggtgcatcatcagctctttggtcctcctgcatctgttcgactccttcaagccccccacccacaccgccgtcctcttctacctgctgtccttctgtaagagcgccacagtgcctctggtcacggttagcatcgtcccgt

SEQ ID NO: 56 - DNA, 426 bp, oryzias javanicus
ttcaggtgcctggctgagggagcaggcgatgtcgcattcatcaagcacacgattgttagtgaaaacaccgatggtaaaggtcccgaatgggctcgcagcctggaatctgacgactaccagctgatctgccctggaaaggctccaatggccatctctgaatttgctaattgtaatttggcggttgtgcctgcacacgcagtagtgacccgtccagagagccgcagtgatgttgtgcgcatcctccaggttcagcagactttctttggagccagtgggagcgatccttccttcaaactcttccaatcagagaatggaaacaacctcctcttcaaggactccaccaagtgtctccaggaggtttctgctggaaagagcttcgatcagtttttgggagccgagtacctcggccgcgaccacgctaatcac

SEQ ID NO: 57 - DNA, 21 bp, Artificial Sequence
  forward primer
aagctaatgccgaggttgatc

SEQ ID NO: 58 - DNA, 20 bp, Artificial Sequence
  reverse primer
ggaggcagaggacttgaatg

SEQ ID NO: 59 - DNA, 21 bp, Artificial Sequence
  forward primer
tcaaccagtggcagataaacc

SEQ ID NO: 60 - DNA, 20 bp, Artificial Sequence
  reverse primer
ctggcacttcctcaaatctc

SEQ ID NO: 61 - DNA, 21 bp, Artificial Sequence
  forward primer
cgatgccaggtccgatgaaac

SEQ ID NO: 62 - DNA, 22 bp, Artificial Sequence
  reverse primer
gcccaacacagccagcacacag

SEQ ID NO: 63 - DNA, 20 bp, Artificial Sequence
  forward primer
aggtgtcccactatcggttc

SEQ ID NO: 64 - DNA, 21 bp, Artificial Sequence
  reverse primer
cttcatcctccagagcttgtg

SEQ ID NO: 65 - DNA, 21 bp, Artificial Sequence
  forward primer
cctggaatctgacgactacca

SEQ ID NO: 66 - DNA, 20 bp, Artificial Sequence
  reverse primer
cctggaatctgacgactacc

SEQ ID NO: 67 - DNA, 21 bp, Artificial Sequence
  forward primer
acaaaaggttccactctcagc

SEQ ID NO: 68 - DNA, 21 bp, Artificial Sequence
  reverse primer
ccaactttaacctccatctcc

SEQ ID NO: 69 - DNA, 20 bp, Artificial Sequence
  forward primer
ggtcaaagcttttgtgactc

SEQ ID NO: 70 - DNA, 21 bp, Artificial Sequence
  reverse primer
tctggcttctccttcttgtag

SEQ ID NO: 71 - DNA, 578 bp, Oryzias javanicus
ctggatgaatatggacctggtgaaacactcaggcggctgtcactgtggagctgttagatttgaagttaggagttctccagatctccacgtcttccattgcaactgcagcatttgtatcaaaaaacaaaaccatcatttcatcgtccccaaaaacaacttcaaacttttacaaggccaggaaaatctgacaacatacacctttaacacccatgcagccaaacacaccttctgtaaaacctgtggagttcaaagtttctacacgccgcggtccaaccctgatggctatggtgttgccccacattgtctggatccaggtagcgtacgcagtgtgacggtggagaggttctgtggagaaaagtgggaggagagcatgcaagctcacaagaccatccgagacatgtccaaactcaaagaggacatgcagcagaaagagtaaacccccaacaaaggcatttattatttcaatctgtgagcatattcaagtacttagtgttcttttacactttgttacattcaagcacaataatgtttttccaaaaaactttaacttttaaaaatgttttacataataaagtatc

SEQ ID NO: 72 - DNA, 385 bp, Oryzias javanicus
gagctccacggggccggacagagcgcccagggaccactacatgacgcggggtcctcagaccaccttcatgaagtctgttcctgattgcttgggtagagacattcacaccagtggccctctgcaggtcattctgaagggcacgagcagtgctcagcctgttcctccttgcacaaaggagcaggtatgggacctgctgaggggttgaggaccttctacggtcctgtccggctctcccagaataaccagcagtctcctgaaatctcctccatgttctggagattgttctgggagacaccttaaaccttcttgcagcagcacgtgtggatgttccatcctggagaagttggacaacctgtacaacttctgtagggttaaggaatctcct

SEQ ID NO: 73 - DNA, 698 bp, Oryzias javanicus
ccgtttgatgcattgtgcaaatattttatgttatagcccttgctctgtttgtttggagccggtacattataatcagggcagggactgttgctactcctctctacacgaccaatcagctgttgggtcaagagtcactgctatattaactctgcagttaaaactgtcttttaattgaaagagacaggagctcggcgaaaacattttttttttaaaactttgcttcaaagcatctcctactgtcatggcaatgcttcttctgaaacgcttggcgaaagagcatgaggattacatcaaacaacaagtccatgtgaaaaatccttatcttgactccatggaggaggacattctgtaccacttcagcctgagtacaaagactcacaacctcccagagatgtttggagatatcaaatttgtgtgtgttggtggaagcgcgaatcgaatgaaggctttcgcccacttcatccaccaagagctaaatgttcctggaaacccagacgacatccaagacatctgtgagggaacagaccgctactccatgtacaaagtggggccagtgctttctttaagccacggcatgggcgtcccatccatctcaatcatgctgcatgagctcatcaagctgctgcatcatgctcactgccaggatgtggttttgtttcgtctgggaacatctggaggagttgggttaaatccaggga

SEQ ID NO: 74 - DNA, 871 bp, Oryzias javanicus
aaacacacacacacgtccccaaacacaaacacatgtccatagccccagaggttgggaaaaataaataaataaatatatggaaaatagaaataaaaatctaaatagaaatctggctttgtactgaagtgaggaaacagtattttaggaatctgtccacaccagtgacccccaaataagttcacagagtacactactataggaccccccaggtcctagcaaagagtcagacccgggaggtcccactgcagcgaccccgttcactgacaaaggacaatcactgatgtgaaggatccctcagaggaagacactggagttaaaaatgtaaaaggagtaaaaaattaaaaataaataaaaacaggtataaataaccagacagataaatagatcaaaaagacagacataaactagtaaaactaagtaaatatcaactaggtaaaaagcataatgagagaacataaaacaacataaaagcatgaattaaagtataaaataactttctagatttcaactaaaagccagattaaaaaggtcggtcttgagccatccttctctgcggccctgaggctctctggaaggctgttccacaggcgacgtccataatgactgaaccagcgtccctaaacatgctagctttggggtttttttttttgccaattagacatttactgagattttttgggctcttttggaatttggctaatattttttactacatgctcgtcattttggcattttcttaagttttttagacaatttgtagttttgctaataattaagcaacgagctattattttttctcttgctaatacggcaactactggagaaaaattggcatttagctaatattttagcaatttgtcatcctcagctt

SEQ ID NO: 75 - DNA, 888 bp, Oryzias javanicus
gctgttcgaaatggagtaagtcaggctaaagtcaaaaacatccaaaaacagatccaactgacagctgctgtcaatgagacaagtctaaatgttacactgaagacacctcaaagtactctatacaaacttggactgggtctcccaatttctctgccacttggagatactgctgctgagttggaggcttatcctgacagctggactcacaaactgtcctacatgctcaccaaagcccatgctgctcagtgtgtcttggtcaacgacagattgatcacattcaacaacaaaaaatacaaaaatgagatgcctcattcttgctaccaagtcttggctcaagattgcacttcagaactcaaatttatggttctgctgaagagagaccaaactgcacaagagaacgagattaatgtcaagattgaaaacatggacgttgatatttattcaaggaacaatgctaccgttgtgaaggtcaacggagtggaaatccactcttccaacatgccgtatcagcattcaacaggcaacatacagatcagagagaaggatgagggtgtttctctgcatgcacctaaccatggccttcaggaggtcttcttcaaccagactactgttaaggttcaaattgctgactggatgagaggaaagacttgtggactgtgtggaaaggctgacggtgaagtcaaagaggagtatcgcacacccaacgaacgagtgtcgaagaatgcaaccacctttgctcattcctgggttctgtctggacagagctgccgtcacacctctgagtgctacatgaaacttgagtctgtgaagctggagaagcaggtcaaccactttggtgaagactccaaatgctactctgttgagcctgtgcttcgctgtctccccggc

SEQ ID NO: 76 - DNA, 867 bp, Oryzias javanicus
tccatcgctccactgcccaccgactgcggtgtctggctgccggagcaggagctcggctccgccccctgcagacacgccgtctctgtatcactgaaggaacgacagacattgaaatcaaaagagtctgcatccactgacgtgacgcaggacacgaagagctccttagaggtcgagtggccgacgtgagggttcctctgaggggacgaggcggccgcatgcacggagcagccttctgaagcggcggcgctcatcctgggctccggtttaaccagggtcacactggcagctgtcccgcagcagatcgcgggctcacagtccaggtcaaaggagtcttgtctctctatggtgcggaaatgagagcaggagacctctgctggtgtcttctcaggctgacaaagcttaccgtcgggaagcctccccagttccactgcatctggagcccagacgaatcctggatttccaggagatcagagtcactccctggagatgctggctgactgcagaaaacgctgcggaggagatccgtccgttagagaaacctttgtgtttcaattagtgctgccacaaacgagtcgactaatcaccgattattttttcatattagtcgactaatcggatcatgcacaaactggatgtaaagcacacatctgaaccatcattagctttaaactcactaaaatctagatatatagcattaccttccataatgctagagtgaaagctgtcagcggaatttggccgctaaagatgctgaaatagatagctaaaaacactgaagctgatagccacctaaaatattgattaaatgcctaattagcccagaaaaacccaaaaaaggttaagttagccaaaacagctagcatgcag

SEQ ID NO: 77 - DNA, 842 bp, Oryzias javanicus
ggttggagtcatgtcggcagctgctcagggaaccagcaaggcgacccacggcgatgtagcctctaagaaaaagaagggacccggcgccctggctacggcatatttagtcatttacaatgttgttatgacagctgggtggctggtgatagcggtgggtctggttcgagcttatctggccagagggagttaccatggactttattattccatagaaaaaccactgaagttctttcagacgggagctcttctggagattcttcactgtgctgttggaattgtcccgtcctctgtggttctgaccggctttcaggtcctgtcccgggttttcctcacctgggccgtcactcacagcgtcagagaggtacagagcgaggacagcgttctgctgttcgtcacggcctggacggtgacggagatcatccgctattctttctacaccttcagcctactcaaccacctgccatacctcatcaaatgggcacggtacacctttttcatcttgctgtatccaatgggagtgaccggggagcttctcaccatctatgcagcgctgccgtacgtgcagaagaccggactgtactccgtcaccctccccaacaagtacaacttctccttcgactactacactttcctcatcctcgtcatgatctcctacattcccctcttcccgcagctgtacttccacatgctacgacagcggaagaaggtgctgggccggacagaggattacagcaaggtggagtgagtccagcagggatccggatggtttttaagacaaaaaaagggaggaaaaaccccacaaacgaagaacaaaccacatctgcttattaaactcaaaagta

SEQ ID NO: 78 - DNA, 837 bp, Oryzias javanicus
agatattggttctttgttctttctgggaaaacatcctctgaaaaatgacattaaaagccgccagtgtttcaattcttcctttgtcaccttgatgtattctgtttagaagagatgtggacgtccaaaaacggagagaagggaggtgtttgatgataatctgcgatctccgtttgacgcaattcaacatcaccctctgccctgtctgtttaactgagtcgtagactgccagtatgatggctgttctctctgtcagaggcaggaggactgctgatgtaaacacttgaggatctttacatttggttcctctgtttttgaatttctctctggatggaaagtggttgtgaccatctgcatcagtttgatggacgttgtggttctttgtggttgatggacggttcccctgacctgtttaaaaccactgagaagcagtttaagcaggtcaggggaaccagaagtgacagacggctgagtctatgcaaatccgttcacatcagtagcatcacctctgtaagaccttgcagagagatgtggaggaaaaaaaatgttcattgtaaaaattctgctgaaacactgtgtgtcttagtgaataaagaattctcctaacgcttcctgtgcggcaggtgttgtggagggagatctggccgcagtggaagcctacaagtcgtctggcggcgacatcgcgcggcagttgaccgccgacgaggtgcaactgctgaaccgctcctcagcgtttgacgtcggcttcactctggttcatctagctatccgcttccagaggcaggacatgctggccatactactgacagaggtgagccaacaggctgcaaagtgtattcc

SEQ ID NO: 79 - DNA, 793 bp, Oryzias javanicus
tagtttcttccgtttcattttgtggttttcttcgattacacgcatatttttgcaggtgtgacgacagaaatggcccgggggcaccagaagtttcagtcccagcagaaaaacgccaaaaagcaggcagagattaagaaggccaaaggacatgaccagaaagcagcagcgaaggctgctttggtttacacctgcgctgtgtgccggacccaaatgccagacccgaaaaccttcaagcagcactttgaaagcaagcatccgaagtgtcctctgccgccggagctcgcagacgtggaggcgtgacggctccacccactccctgtgaggaatactgtggtcgctggtgtttagtgtttagtatctccaggtgactaattaacctgtgtgggttttctggcatggcactcacctacctgtggaaatctgttaaattaaaatgctttaattatttaattagtttttgccctcactttccagaatatgaaggactgaagaccacccgcccacctgcccacttgcacaatactattgggtggttgcagtattttcaaactgtgttcattccgagcattaaagtgtacatttcaaatctgcaccccccctgcagaaaaacttagcagaagctgcctctctaaactaatctgtgtaagaggaagtgatttagatccagagttctggatgttcagcagaactttattagtcgggtttgacccgatgatttgaaaagaagactgaatcttaataggtttgcaaaagggctgccacgattagtcgactaatcgatgactaatcgact

SEQ ID NO: 80 - DNA, 766 bp, Oryzias javanicus
  misc_feature (657)..(657): n is a, c, g, or t
ccatggcagctccgaagaaagtctgcatcgtgggctctggaaactggggctccgccatcgccaagatcgtggggaccaatgccgttcacaatccaaagtttgacaacacggtgaaaatgtgggtgtttgaggagatggtgaacggcagaaagctgacggaaatcatcaactccacccacgagaacgtcaagtatcttcctggacacaaactgccggagaacgtgctggcagttccagatctggtggacgcctccaaagacgcagacatcctgatcttcgtgattccccaccagttcatcggtaaagcgtgcgactccatgaagggcaagattaaaagtgacgccctggggatatccctcattaagggcgtagacgaaggccctgacggactcaaactcatttcggacgtcatccaggagaagctcggcatcgcgatgagcgtgctgatgggagccaacattgccaatgaggttgcagacgagaagttttgcgagacaacgattggctgtaaaaacaagaaccacggatctcttctgaaggagctcatgcagaccaagaacttcagagtaactgtagtagaagaacttgacgtggtggaaatctgcggtgctctgaagaacattgttgcggtaggtgcgggcttctgcgatggcttgngctttggggacaacaccaaagcagccgtgatccggctgggattgatggagatgatcgccttcgcccgcatcttctgcaccgccggacccgtgtcctccgcaaccttcct

SEQ ID NO: 81 - DNA, 913 bp, Oryzias javanicus
tttggattagtctgttcctgtcagccattttagacttttccatttccatgttaacaagcaaccctattacactggagccaattaaaggcctccgtaggctgtcacccactgtaatcacttgagagccagaatcccacaaagatcttggtttctcttggatgtatcagtctaataatgttttaagccaattagaagaacagtctattttatttatctctttgtggggttttggatccaatcatcccatgaagtttgtgcacaataaattttgagagcagtccaattttcactctggcagaacttttctatatgacagttggatacagtcgacatttaacttttgcagcctctcctgccaccgtctgcgcagtgttgagacactggtgtgtggtgatcgatccccatcctgctgaaacgaatccccttcatttaaagcaggggtctcaaactcgcggcccgcgggcaatctgcggcccgcgggatgataatttgcggcccccgtcttcatatgaaagattactgttagtgcggcccgcaaggctgatatgaatgacagttgttgtgtccagagctgaacgaaccaatcacggtaaggtatatggttcttgagggcgggacatcagcgggctgtccgagcctcctcactctttaattcattcctccagtcagctggacggaggaggagccatgaagctgctggaagagctttctcactgaacgggattcgcgggccccgctcctctttcaccacgtgacaacacgtgaacacagcctgccgcttgtcaaaaagcgtgttccacgcgacaattccgcgcacgcatcaaatattccgcgcggacaaatcagaccctcgcgagcgctttttcctcctccacgagcagaaaaagtcctcgcgagagagactgactacttc

SEQ ID NO: 82 - DNA, 795 bp, Oryzias javanicus
tgaggtctctggtcttcgttctgctcattggagccgccttcgcccttgatgacgacaagatcgtcggagggtatgagtgcaccccctactcccagccccatcaggtgtccctgaacgccggttaccacttctgcggcggctctctggtcaatcagaactgggttgtgtctgctgctcactgctaccagtcccgagtgcaggtgcgtctgggagagcaccacatcatggtcaacgagggatccgagcagttcatcacctcctcccgtgtcatccgtcaccccggatacaattcctacaccattgacaatgacatcatgctgatcaagctgagcactcctgccaccctcaaccagtacgtgcagcccgtgtctctgccctccggctgcgctcccgctggcaccatgtgcttggtctctggatggggtaacaccatgaaccccgctgatgatggcgacaagctgcagtgcctgaacatccccatcctgtctgactctgactgcaagaactcctaccccggcatgatcaccaactccatgttctgcgctggatacctggagggaggcaaggactcttgccagggtgactctggtggccccgttgtgtgcaacggccagctgcagggagttgtctcctggggatacggatgcgctgagagggaccaccctggtgtctacgccaaggtctgcattttcagcgactggctggagagcaccatggctagcaactgagtgatccaactaaaaatatgaagtccagcagctttacataattagctgcttgtattccatctgactt

SEQ ID NO: 83 - DNA, 859 bp, Oryzias javanicus
acagtttgcgcagcggcactccggtggctgacgaacctgctgctgcttctggcttgtttgcggcatttgggacagaaacggggcggtttgagactcggtgacggaagcaggctgtcagtgcgggaccgagcggctggactccgcgcagggggcgttcctgtgaggccagaatgtggggcatttggagcaaaacggcggtggggatggtgaaagtgtgtggattcgtgagacctcaccacttcgtcaagccggtgtcagcgactcgagtgtctgggagataccgctcacatcagcaggggttgcccactctgcctgtgccccctctgcagcagacctgtgagcgttacatcgctgccttagaacctatcacagatgtcgacgacctgacacgcacaaagaagttggtggaagagttccagaaaacaggagggattggagaaaggctgcagaaaagcctcgaaaagagagcatgcactgcagaaaactggttgtccgactggtgggttcaagtcgcgtatctggagtaccggatgccggtggtggtccattccagccctggattggttttgccgcggatgaatttcagcgatcagaaggatcaaatcaggtttgctgccaaacttatagcaggtgttctggactttaagacgatgattgacaatgagactcttccagtggagtacatgggggggaagccgctgtgtatgaaccagtactatgaagttctgtcttcctgccggatccctggtctgaagagagattctgtggtgaaccacgccaagagttcccacccccccagacacattactgtggtacacaactttcagttctttgagctggatgtgtatagcagggatgg

SEQ ID NO: 84 - DNA, 771 bp, Oryzias javanicus
gctggtcagtgttccctctgttaaaagttcatctctactaactaacatccatcatgagggaaatcgtgcatatccaggccggccagtgcgggaaccaaattggtgctaagttctgggaggtgatcagtgatgagcacggcatcgacccgacaggaacgtaccatggcgacagcgacctgcagctggacaggatcagtgtgtactacaatgaagccacgggagggaagtacgttcccagggccatccttgtggatctggaaccaggcaccatggactctgttcgctctggacctttcgggcaaatcttcagacctgacaactttgtcttcggtcagagtggagctggaaacaactgggccaaaggtcactacactgagggggcggagcttgtggattccgtcttggacgttgtgaggaaggaggcggagagctgcgagtgtctgcagggtttccagctcactcactccctgggcgggggcaccggctccggcatgggaaccctgctgatcagtaagatcagagaggagtaccccgaccgcatcatgaacaccttcagcgtggtgccttcccccaaggtgtctgacacagtcgtcgagccgtacaacgccaccctgtcggtccaccagctggtagaaaacacggatgagacctactgcattgacaatgaagctctttacgacatctgtttccgcaccctcaaactcaccacgcccacctacggagacctcaacccactggtgtcagccaccatgagcggcgtcaccacctgcc

SEQ ID NO: 85 - DNA, 860 bp, Oryzias javanicus
tgtgatttatttttctgtccgaaatcatcgtttttcactttcgggtttgccatatcaaataaatgtacagatttaacctttaagtttagggggaggagccatggatcctgttcttcttttagggtttttttcagtagctaaaacttcctggattgtaaaaatgtttttatgagagatttatatttttcgttgttgttttttcactttttcaaatcgttgttttggataacggagcggctccgcagggcgacgctcctccgcggcggcgtttttgtcagaaagtttggggaaaagccggagcgctttcgctccgttcgggaagttttgagggaagcgttgggaagtccaggtgggaaagaagtttgggtgtggaagtgatgtgaaaagggattttcagagatggctgtatgcaataactcgcttgggaattccgtctggtttgggaaaagtgcctgttttggttggtttggatgcaggaagggatccggtcggcttttttctttcacgttaaaacgttccgttgtcgtcgggatggtcgttctctcagatgtgggatatatttttctacgtgtttcgcttttggtcgtctggaaacgggtgatgaggctgaggtgctttagcatttatgaggcttttcagaataaaagaccgttaaaaaaaaggtttttcatcaaagtaaaagctcaaataacaccatatccaaactactatgattcttatttaagttttgtggctgtaattgatttctttaataggccgtttgtgtttctgttgctgaactttttctttttttgtagttcacactgaatttcttttttttctgttttcccgccttttatttggacaaagttctgtcagcc

SEQ ID NO: 86 - DNA, 767 bp, Oryzias javanicus
tcccctctccaaattggggtggagctgaccgactctccctacgtggtggccagcatgagggtgatgactcgcatggggaaggcggttctgtccgctctcggaagtggcgagtttgttcgctgcctgcactctgttggatgtcctctgcctcttaagaaacctctggtgaacaactggccctgcaaccctgagcagacgctcatcgctcacatcccagatcgcagacaaatcatctccttcggtagcggttacggtggaaactctctgctgggaaagaagtgcttcgctctgcgtatcgcctcacgcattgcaaaagaggagggctggctggcagaacacatgttgatcctgggcatcacaaaccctgccggagagaagaagtacatggccgcagctttccccagcgcctgtggaaagaccaacctggccatgctgtgccccacattgcctggctggaaggtagaatgtgttggagatgacatcgcctggatgaagtttgacaaaaatggtaatctaagggcaatcaaccccgaaaacggcttctttggcgttgcacctggcacttcagtcaaaaccaaccccaacgccatggcaaccatcaccaagaacaccatcttcaccaacgtggcggagaccagtgatggtggcgtgttctgggagggaatggatcaaaccctgcccgagggagtcactgtcacatcctgggagaaccagacttggacttcaaaggatggagagccctgcgctcatccaaactctcggttctg

SEQ ID NO: 87 - DNA, 725 bp, Oryzias javanicus
gggactctcactctgacagaggacgatcttcggggcatattcaacataggaggtcccctgtctgtctcttttcactcctaacgatccgcgcaggctaaaagaaaccgctggattgaggttgcggtctgtttgtgcgtttcccccatcctgtctcggagcaaggatgaggataagggagctttctttacgcccggatcctgacctgaggaaggagctggccttgctggccacgaggatgtgactttgttctgccttctcggtttgagaagaggttaatagcctttcatgaggacaggcccgggtgaggacggaagtgccggtcaccgcagcgcacagcgaagacattcccaagttctacttcccacatggccggcccccagccaacctccacatcatactgcctcatttccggagttgagaaaatattttcccaattcccaagtgagagggccaccattgaggatatgggggcggatgccaaggtctgtgagtgtcccctctactggaaagtgccattgatctgcttggctggaggcgacaagaccggtttcgtgtctgttcaccagtttgtggctgtgtggtgaacaactctgcagacctgccacgatgatgcttctaaatttgtgcatctcttggccaagtcgggctgtatttacctggaacaagatgactgtttttcattcctgcacgacgttgtgaactcgcacgcgagcctggcctttctg

SEQ ID NO: 88 - DNA, 903 bp, Oryzias javanicus
aaagccaggcttaacaaagctatgggaacttctccaaaccggctgcccagagtctgaaccccaccagcgacggattgcctttcagccaaactttaaacccctcccggacagctcgtgcggaaaaagcgtcgtcaactctggaagaagcagcagcagcagcagccatggactcgagagacacagccccggattcatgggagctggaggaggatggcgaggccccggctcccgcagcggagctcccgaccgccttcgccggcctcaacgtcaacgccaagccgttcgtccccaacgtcaacgccccggagttcgtaccgagcttccttcaagccagctcggcggaaatgccggcctcagacggttcccctgaccccgctgctagcatggaggtggcagacgcagccgccccggtggagaacggaaactccgaggcggacatgaacacagaggaggagacgtgggagcagaaggaggagcccgggggaggcgaggggggcggaggccaggagaacctgggcacggagggaggcgcagcggggagagaagacgaggagatgatggaggaggaagaggaggagatgcccgtaccaaaggtggctccgccgccgcccgacgcgcccaaaaaggagcacgtgaacgtggtgttcatcggtcacgtggatgctggaaagtcgacgatcggaggacagatcatgtacctaactggaatggtggacaagagaactctggaaaaatacgaacgcgaagcgaaagagaagaaccgggagacttggtacctgtcgtgggctctggacaccaaccaggaggagcgggacaaggggaagacggtggaggtgggacgcgcttactttgagacggagaagaaacacttcaccatcctggacgcgcccggacacaagagct

SEQ ID NO: 89 - DNA, 496 bp, Oryzias javanicus
gatgtctctggtaatccccgagaagttccagcacattcttcgtgtactcaacacgaacatcgatggtaggaggaaaatcgccttcgccatcaccgccatcaagggtgttggcagacgttacgcccatgtcgtcctgaggaaggctgacatcgacctcaacaagagagctggagagctaacggaggaggaggtggagcgtgtggtgaccatcatgcagaacccccggcagtacaaaatcccagactggttcctcaacagacagaaggatgttaaggacggcaaatatagccaggtccttgccaacggtctggacaacaagttgagagaggatctggagcggctcaagaagatcagggctcaccgtggtctcaggcacttctggggtctgcgtgtgcgtggtcagcacaccaagaccaccggccgtcgtggtcgcaccgttggtgtgtctaagaagaagtaaactcttcattttgtttcaataaaacaacaacaaaag

SEQ ID NO: 90 - DNA, 766 bp, Oryzias javanicus
tgcgtctgggagagcaccacatcatggtcaacgagggatccgagcagttcatcacctcctcccgtgtcatccgtcaccccggatacaattcctacaccattgacaatgacatcatgctgatcaagctgagcactcctgccaccctcaaccagtacgtgcagcccgtgtctctgccctccggctgcgctcccgctggcaccatgtgcttggtctctggatggggtaacaccatgaaccccgctgatgatggcgacaagctgcagtgcctgaacatccccatcctgtctgactcttgccagggtgactctggtggccccgttgtgtgcaacggccagctgcagggagttgtctcctggggatacggatgcgctgagagggaccaccctggtgtctacgccaaggtctgcattttcagcgactggctggagagcaccatggctagcaactgagtgatccaactaaaaatatgaagtccagcagctttacataattagctgcttgtattccatctgacttacaaaaatataaaatatttgaagctgaaaaaaaaaaaaaaaaaactcgagacaaccagaaaggattctgctcagacgcagttactctgcaacgatgcaaacaaatcttctcctggtgactctttgtctcctcggttcagtaatcctctgtggagggcaggatccctcaagtacaactgctcccaccaccgcaacagaaagcagtactctcagtcaagcacctccaactactgactccatcccaaccacaa

SEQ ID NO: 91 - DNA, 833 bp, Oryzias javanicus
caccagagaggcccccgtccccaggacttctgccggccatgtacagcccccctccaggcatggacagccacaccgtctgcattccctctccgtacacggatagtagccatgaatatggcaacggacctggacctttgaccttctatagtccatctgtgctgagctacagcagaccaccgatgaccgacagtccctcctcgctgtgcccccccctcagtccttcagccttttggccctctcatggtcatagcgtgccctccctgactctgcattgcacccagcccatcctctacaatgagcccaactcccataccgcctggctggaacccaaagtccacaacatcaatcccaacagctgcaacaagctgctggggaggaaatcggacataggagtggaagacatgaaacccccctcgtgttctccctctgcagggaaagctgacatgcacttctgtgctgtgtgtcacgattacgcctctgggtaccactatggggtttggtcgtgtgaaggatgcaaggctttttttaagaggagtattcagggtcacaacgattacatatgtccagccaccaatcagtgcaccatagacaagaaccgccgtaaaagctgccaggcctgccgcctgcgcaaatgctatgaagtggggatgatgaagtgcggtgttcgccgtgaacgctgcagctacagaggagctcgacaccgccgaggggggctccagcctcgggacgcagcagggaggggcttggtccgggtcaggatgggtcctcggacccagcgccatttcagtctggggatacctctttccccgttgccagggggccctcccatccct

SEQ ID NO: 92 - DNA, 880 bp, Oryzias javanicus
tggaacctacaaaggtgaccctttctactacctgaaagagaagacgagttcccatcatgcagttctgggcggattcctcaccattctgaagagtgagacctgtcagggagctccagattctgtttttgactgtgttgcctttgttggggtggccgtggtgcagcggtagagcagtctgatcagaagattgcaggttccatacacgccttacccgtccatgtgttgaggtgcccttactgaagatttggtggaaggttggtgccagtgttccacagtggagccaccctcagtgtgtgaatgggactgcaaagcgctttgggccttcaaggaaggattatcaccatcatcatacaagccgacctgagtccgcctctccactactgcagtcctgattcacacaggaaccctgcctccagtcacgccacgtccccacgcccactactcacctctgggctctgctatatattggaaccaaccaccctggaacgactcagctgctccagccagttgtggggggaaactggtcccatccctgcctgagtgccattctgaaagagacttaagaaactttagttctttctttccataagaggtttattgagagtttggggtgtcaagccccaaaacgcatagaaatcctactttcatcatataatttccatccattttctaaacccgctatatctcttttggggttgccgtatcctgtcacggctactgttggctactcctgctaacactacaactactttatttaccgggcttgggaaaggcccaagccattggtttgtgccctgttagggctacatttatcttttttcttgtctctatgtgtgtttgtccattctcatgttgtgtttttgtcttttattcatgtatt

SEQ ID NO: 93 - DNA, 774 bp, Oryzias javanicus
tttgctcagaagtgtctgatccagcaactcgatcaacatgtctatgactctgggatactgggatattcgtgggctcgcccaccccacccgccttctgctagagtacaccggcaccaagtatgaggaaaagacctacgtctgtggagaagcaccaaactttgacaaaagcggctggtttagtgtgaaattcaaacttggactggacttccccaatctgccctacttggtggatggcgacaaaaagattgtgcagagcaatgctatccagaggtacctggccaggaagaacaagctttgtggagaaactgaagaggagaagattcgactggacatgctggagaacgtggtcgtggacttcagaatgggcttcatcaggatgtgctacaccgactacgacaacctgaagccagagtacctcaagacactccctgatgtcttgaagcagttctcagagttcctgggaaagaggaagtggtttgctggtgacaggctcacttttggagacttcataatgtacgatgtgttggatcagcagaggatctttcttcctacatgcctcgatgcctttaagaacttgaaagacttcctggaccgctttgagtctttggagagaatttctgccttcatgaagtcaggcagatacattcggaccccgatccacaacaggatggccaagtggggaaacaagaaaatgtgaagactcaagtttcataaactccagaacagcagtgctactgactcatataaacaacacttctttgtatttgttattta

SEQ ID NO: 94 - DNA, 867 bp, Oryzias javanicus
gcgcacagagatccccccctctgcagcacagagatggagcaaaccgagctggtgaagcccgaaactctggacgagctgatcaaaactctgcaccagatcttccagagtgaccgcatcaacgtggaggaggtgcagaacctcatggaatcctatgaaagcaagcctcacgagtggaggaaatacgcaaagtttgaccagtacaggtacacgaggaacctggttgatgaaggcaacgacaagtttaatcttatgattctctgctggggtgaaggtcacggaagtagcatccacgatcacacaaactcccactgtttcatgaagcagctgcagggccagctcaaggagacgctctttcagtggccggacggcaagtcgcagggagacatggtcacaaagtcccagagagtcctccaagaaaacaaggttgcatacatcaatgactccatcggtctgcatcgcgtggaaaacgtcagccacacagagtgcgcagtgagtctgcacctgtacagccccccgtttgagagctgccagacttttgaccagcggacgggacacaagaacacggtcaagatgaccttctggagcaaatacggagaaagaactccatttgagaccacagtttcgcaggaaaacaactaacggaggacgacgaagacacgcatttgaaaatgtcatgaagctgttttctcggaaactgctggaggggagcaaagtgtggaccaaccaacgacgacaaagcaacccattttcaaatcaagagtgggcttgttcaaaaggacaacccgctaattagaatggctttatcgctgctcagtgtgagcttggcaaatactgatgagcacaggacgaatgcatgaatgggctcgg

SEQ ID NO: 95 - DNA, 779 bp, Oryzias javanicus
  misc_feature (687)..(687): n is a, c, g, or t
agaactccagcggggtgtctgcaaagtgcagagacagccgggtggaggagctgtggggtctgaccactttctttggctacagcacccacaccttcgttcaagctgtcaatctgcttgacagatttctcaccttaatgaaggtgcagcgcaaacacgtcccctgcatcggggtctgctgtctccacatggccgccaaaatgatggaggaggaggcagacgtctcgccgacccacgagctcatccgcatcagccagagcaagttcacggtgtcggacctgagccgcatggagaagatcatctcggagaaactgtgcttggaccccgaagcggtgaatgcgttgaccttcctgcagctctaccacgccgccatcagcgctcagtctgccggagggtaagcgtcttctccgtcccgctgccgtttgatccactttgagctggtcacagaggttgttctttatgaggtctgtgcggcggatcacgtaaagttcccctatatatttgaagggaggagatcccaagctttgggagactggaagcccagcttaaagcctgtttatgccggcttgttttctctaaagcaaaagtaagacttgttctcagctttgctgccgtcgccgcccgcccgggagctaacgtggcgctccttcttcacagccgtccgtgttggcgctgtcgctcatcgctcangacccggacggccttcagtcgctctcggtgatggagattgtccaacagtttcagaggcatctgaaagtaggttttctcatttccccttttgg

SEQ ID NO: 96 - DNA, 804 bp, Oryzias javanicus
aaagtggtaatcagagatgctgatgtaagtgtcactgtcatttttcttcctgacttattaatgcatgagagcatctttctcctgcaaaccccacacaacggactctactcccccaaatccaggaccatgtctgaaatctatgatgattctggcagtttggaagagaccaccgacagcttctgggaggttggtaactacaaacgaacagtgaagcggattgacgatggcaatcgactctgcaatgacctgatgaattgcatccaggagcgggccaaaattgagaaagcgtacgctcagcagctgactgaatgggccaagagatggaggcagttggtagacagagggcctcagtacggcacagtggagcgggcttggatggccctgatgacggaggctgacaaggtgagcgaccttcaccaggaagtgaagaacagcctgatcaacttggactttgagaaggtgaagaactggcaaaaggactcgtaccacaagcagatgatgggaggattcaaggagaccaaagaggcagaggaaggtttcaagaaggcccaaaagccctgggccaaaaagttaaaggagctcgaggctgccaaaaagtcttaccacatgacctgtaaagaagagaggcttgccgtcactagagaggcaaacagcaaggcagaggcctcggtgacggcggagcagcaaaagaagctgcaggagaaagtagacaagtgcaaacaggactctcaaaaggcgagggagaagtacgagaaggctctggaagagctgagcaactgcacccctcagtacacagaaagcatg

SEQ ID NO: 97 - DNA, 853 bp, Oryzias javanicus
tggacaacagtaactgctggcagccagtgatcagttacattgacagcaagtgtgaggactttcttaacgctgagtccagggtgaaccgcagatcaatgccggacaacagggtgcactgttgcctgtacttcatcgcaccctctggacacggtttaaagccccttgatattgaatttatgaagagactccatgataaagttaacgtcatccccctcattgcaaaggcagacaccctcaccccagaggagtgccagctctttaagaagcagataatgaaggagatccaggagcataagattaaaatctacgaatttcccgacacagatgaggaggaggacaacaagcagatcaggaagatcaaggagaagatgccgctggcagtggtgggcagcaacgtggtggtggaggtgaacgggaggaaggtcagaggtcggcagtacccctggggggtggcagaggtggaaaacggcgagcactgtgacttcaccgtcctgcgaaacatgctcatccggacacacatgcaggacctgaaggacgtgacgaacaacgtgcattatgagaattatcgaagcaagaagctggctgctgtcacctgcaatggggtggacacctccaagaggaacgggcagctgaccaagagccccctggcgcaaatggaggaggagcgcagggagcacgtcatgaaaatgaagaagatggaggcagagatggagcaagtctttgagctcaaagtcaaggagaagaagcagaagctaaaagactcagaggccgagctggaacgacgccacgagcagatgaagagaaacctggaggcccagtacaaagagctggaggaaaagaggcgcgtctttga

SEQ ID NO: 98 - DNA, 816 bp, Oryzias javanicus
agactttcaggtgaaccaagagcagacgatgaagagtcttgtacttttgctgtttgtggttggagccggcgctaaagtatttgagcgctgcgcgtgggcccgcttgctgaaggagagcgggatggatgggtatcgcggtgtgagcctggccaactgggtctgcctgacgaaacatgaatcaaatttcaacaccagtagtgtcaaccacaacagggacaggtcaactgattacggcatctttcagattaacagtcgctggtggtgtgacgatgcacagaccccaacagccaacgggtgcaacattccctgcagcgcgcttctaaccgacaatgtggccatagcaatcaactgtgccaaacgtgtcgtcagggatccgagcggcatcgcggcgtgggtggcttggcgtgtacactgcaaaaacaaagatgtgagttcctacatcagcggatgtaaattgtaatcatctcacatgaatttacactgcaataaacacaagtatgaatctaaaaaaaaaaaaaaaaaaaaaaaaaaaaaaacctcgaagcccccaccaccgccggcccacgttaccatctagcaccgggaaaaatagcagagagccgccgccggcagaagccattaccaactagtagtaactatgagcagcgaggccgagacacaacaacaaccaccgcagcctgctgccgatgcggaaagcccatccagcccggcagctgcagcttccgcgggggataagaaggtcatcgcaacaaaagtcttgggtacagtcaaatggttcaacgtcaggaatggatatggtttcatcaataggaatga

SEQ ID NO: 99 - DNA, 781 bp, Oryzias javanicus
tcagatgtttaacgcgaaattcatccctccaaactcaagggacggatagagaaggggattactataaaagcaggcagatggatccactgaagaacaagcaaaactctccattcagcacccctgtgtgtgtgcacaatccacatctggagtctttgaaagatgacatcctctaccacttcagccttggaactgagacccatgatcttccagcaatgttcggggatgttaagtttgtgtgtgttggaggcagcccctggaggatgaaagccttcacggagtacattgctgctgagcttgcaatggaagatcccaaatcagagtatcctaacatctgtgctggaactgatcgctatgccatgtacaaagtcggacctgtcctctctgtcagccatggaatgggcatcccctctattgccataatgttgcatgagctgataaagcttctctatcacgctcactgcaaagatgttacgattatacgaataggaacatcaggagggattgggcttgaacctggcacggttgttgttaccagacagtctgtggatgccacctttgagccgaagtttgagcaggtgatactgggaaaaacggtggtgcgcagcacagatctggaccaaagcctggctgaggaactgttgcagtgcagcaaagagctgggccggtttgagacggtgataggaaacaccatgtgcacactggacttctatgaagggcaggcccgtttagatggtgccttctgctcctactctgaaaaggacaagcgagactacctcatcaa

SEQ ID NO: 100 - DNA, 602 bp, Oryzias javanicus
aaaaagaaatcactttagtcaaagagaccgaatcccatgtcgtcatcggactcctcggactcctccttcttctcttctttcttttcctcctccttagctggggcgtctccagcagcagcagcagctccagccgccggcgctccggccggagctccgcctccagctccgacgttacagatcagactgccgatgtcgatgctggagagggcctttgcaaacagcgttggccagaacggctccacggtgactccagcagccttgatcagagcgttcagcttgtcggcggtcacggtgacttcatcgtcatggaggatgagagcggagtaaatacaggccagttcggacacagacgccatcgtagatgagagttcgaagaactgcgggctcggatgcctaaatgtgacggtgctagtcgccggattgaaggccttagcttcggatgaagaaccaaacaccttaggcacgctgggtttgactttaattccagcggcgttaattgcagacgacggccactacaggaacagatgacatcacatataaaaaatgaagctctgaagaacgtatctccagagtatcctgaagaaccacacggctcacgcttg

SEQ ID NO: 101 - DNA, 794 bp, Oryzias javanicus
ctctctcagtctgccgagcctctgtcgctgcagtcttctctgtctgactcaccccatcttcatttgtctgcaaacatggccgaggcgtttgtgggaacgtggaacatgaaggagagcgagaagttcgacgagtacatgaaggctttgggggtgggatttgccacgcgcaaagtggggaacctgaccaaacccaccaccatcatctccatggacggtgatacggtgacggtgaagacccagagcaccatcaagaacacagagctgtctttcaagctgggggaggagtttgatgagaccactgcagatgacaggaaggttaagtccattgtgacaatacaagatgggaagctggttcacgtgcagaagtgggaggacaaagagaccagtctggtccgagaagtcaacggaaacaacctcacactgacgctcacactcggagacgtagtttgcacacgccgctacgagaaggcagagtgatccccgttcctcgggcacctacagaagcactcgcatccgccccaaaaactcccttctgccacttgaaaggccaaaaaacgcccccacactagaatggctgctcgttttatttttttcttactgtacgctcagtattaactgctgactgtgacatccctgttctgttatattattccttttactttaagaaccactgaataaattccaatgagtacatttttgttgtttttcctcaaaaaataatttgagaatggtgcataaatgcctttggaaagtgttaaaatttgaatactactaaataaaagtatccaaaactg

SEQ ID NO: 102 - DNA, 762 bp, Oryzias javanicus
tatgctgtatggccatggcgatgaggcttatacatttctgtcttttggcagttgctctgcatggccatctggcaaatggtcagggtgacaaggtaggtcatcagcctcagtatccttcatatcctcagaagcctcagaatcctcagcaacctcagcagcctcaatatcctcagcaaccccagcagcctcagtatcctcagcaacctcagcagcctcagtatcctcagcaacctcagcagcctcagtatccccagaagcctcagccacctcagcagcctcagtatccccagaagcctcagcaacctcaacagcctcagtatccccagaagcctcagcaacctcagcagcctcagaatccccagaagcctcagcaacctcaacagcctcagtatccccagaagcctcagcaacctcaacagcctcagtatccacagaatcctcagcaacctcagtatccccagaatcctcagcaacctcagtatccccagaatcctaagcagcctcagaatcctcaggtgccaacatcaacatttcacacttgtgaagtggatgagcaccataagataccatgtggctcctcaaacatcacttcctccgagtgtgatgctataaactgctgctttgatggatacagctgctactacggcaaatatgtgactcttcagtgcaccaaggatggtcagtttatcatagtaattgccaaagatgctacactaccccacattgacttgaagacagtcagtttcctgggagcaggtgctg

SEQ ID NO: 103 - DNA, 655 bp, Oryzias javanicus
ggaatggaatgatcttgaacccccatttccacaaagactggcagagaagagttcgcacctggtttaaccagcctgccagaaagatccgcaggcgaaaggctcgtcaagccaaggcgcggcgcattgctcctcgtcctattgctggaccactcaggccacaagtcaggtgtcccacagtcaggtaccacaccaaggtccgtgccggacgtggcttcactcttgaggaacttaaggcagcaggtatccacaaaaagacggcccgcaccattggcatcgcagttgacccccgccgtcgcaacagatccacagagtctcttcaagccaacgtgcagcgcctgaaggagtaccgctccaaactcatcctgttccccaggaaggcctctgcacccaagaagggagacagctctgaggaggaactgaagatggccacacagctcactggtcctgtcatgcccatcaaaactgtgtacaagaaggagaaggctcgcgtcatctctgaagacgagaagaactttaaggctttcgccagcctgcgtatggctcgcgccaatgctcgtctcttcggcattcgtgctaagagagccaaagaggctgccgagcaagacgtggagaagaagaagtaaaagcagtgtgaaactttgcaaaataaagtctt

SEQ ID NO: 104 - DNA, 718 bp, Oryzias javanicus
acggcttttgtctctggcgccggagacttcgttcaagcaggtaactcagaatgaagcgagtctgggtggtcgggcttctctgtgcacttctggcatttgctgctgtgagagctgacgatgaagttgacctcgatggaacagtggaggaagacttgggaaaaatgccgggatggttcctgagcagatgacgaggtggtgcataaagaggaagacgctatccagctcgatggcttgactgcgccgcaggctgaacagttgagacagaactcccaccaacgtgcctttccagctgaactcatccgtatgatgaaactcctcattaactctctctaccaacacgaacagatcttcctcttgcagctgatttctaatgcctcacatgctctggacactattcgcttgctgtctctgaccaatgaggatgctttggctgccagtgaacaactgagcatctcgatcaaatctgataaagataatagcatgctccacctcactgacaccggtattggaatgaccaaagaggagctggttaaaaacttgggcaccatcgcttagtccggcaccagcgagttcctcatctaaatgacggagatgcagtctgagggccagtccacctcagacctgattggacagtttggtgtgggattctactctgctttcctcgtcgctgacaaggtcatcgtaacatccaagcacaacaatgacactcagcacatct

SEQ ID NO: 105 - DNA, 698 bp, Oryzias javanicus
gccttctccgtcacaggaaacatcgagggtcagtggttcctgaaaaatggaactctgctgtcgctttcagaacaagagctggtcgactgcgacggtttggaccaggcatgccgaggcgggctcccgtcaaacgcctatgaagccatcgagaagctgggcggtttggagaccgaaaccgactactcctacacaggaaagaagcagaaatgtgacttcaccaacaggaaggtggccgcttacatcaacagctctgtggagctccccaaagatgagaaggagattgcagcttggcttgctgagaatggacccatctctgttgccttgaatgcttttgcaatgcagttctacaagaagggtgtttctcacccatggaagatcttctgcaacccctggatgatcgaccacgccgtcctcctggtgggatacggcgaacgtaatgggatcccattttgggccatcaagaacagctggggcgaggattatggcgagcagggttactactacctgcatagaggctccaatgcctgtggcatcaataagatgggctcatcggctgtagtcaactaaacacatccagtgacacaagcaaacaaacatggaaacatactaacaccgcagttgcagctgttctgcttcgtcatctttacacaacgcatgaaccttaaaatccaacactcgaaggttttactgattccgtt

SEQ ID NO: 106 - DNA, 895 bp, Oryzias javanicus
gagcttcgtcggccgtcatggtgaaccagcgcgtggaggcgggctggcagagctgcaggagcgactccagcggcggcagcaacagcagcggggagagctccaaggacagctcccgctgctccacgccgctgccggacggagaccgcgcggaccggctgcgggacaagatgcggaggaggaccgagtccggagaccgctggttctcgctggagttcttccccccgcgaaccgccagcggggcggtcaacctgatctcccgattcgaccggatgggcgccggaggaccgctgttcatcgacgtcacctggcatccagcgggagaccccggctcggataaggagacctcctccatgatgatagccagcactgctgtcaactactgcggcctggagagcgtcctccacctgacctgctgcaaccaaacccgggagaagatcacgggttacctggctaaagccaaacacctgggtctgaagaacatcatggccctgagaggaggtacggatgaagtgttcctcagcatccagactcacctttaaagacccttttaactctttgactttaaatgatcaaaggacagttttctgtatcttttgattgattgtaaaatagtttccagagatctttgaattatcataaaaggcgggatctttccaggacatagttcctgcagagcggcaggagtttattgtgggttggactgttggtgtagaagtgagcccgcactcatttcccatcatcctgttgtaaacaacagggaccttaaactggcggcccgcgggccattttcggccccctgatggtattttacgactcccgccttaatatgaaagttcaatgtctactaatcaataccttctttgatgttagctctgtgtttgctttgtgatgtttc

SEQ ID NO: 107 - DNA, 725 bp, Oryzias javanicus
aacgcgccgctcgctcccgcgcgtacagctcagcagccgtgcgctcgctcctgtccgcagacccgtccacggagccgaaccttccggaccgagtccgcgccgcctccgcccccagagcccccgaagcgccgctcctcatcatggcgacagctgcggcgtcggcccccgccggctgcggccccagccccggcgcgggcacggagctggtccgcgggcaggccttcgacgtggggccccggtacagcagcctgtcctacatcggggagggcgcgtacgggatggtgtgctcggcgtacgaccgcgacaacaagatccgcgtggccatcaagaagatcagccccttcgagcaccagacctactgccagcggacgctgcgcgagatcaagatcctgctgcgcttcaagcacgagaacatcatcggcatcaacgacatcatccgcgcgcccaccatcgagcagatgaaggacgtatacatcgtccaggacctgatggagacggacctgtacaagctgctgaagacccagcacctgagcaacgaccacatctgctacttcctctaccagatcctgcgaggcctcaagtacatccactctgccaacgtgctgcaccgcgacctgaagccctccaacctgctgctcaacaccacctgcgacctgaagatctgcgacttcggtctggctcgagtggcggatcccgaccacgaccacaccggcttcctcacggag

SEQ ID NO: 108 - DNA, 615 bp, Oryzias javanicus
cgccgttctagtaaaaaagcagacagcctgtttaactcgacgtgttgtttgttgtttgtttgtttgtttgtttgtttactgaccccgtccggaagcggccgtcctcatggcgtaccagctgtaccggaacaccacgctgggaaacagcctgcaggagagtctggacgagctcatccagacccagcagatcaccccccagctggcgctccaggtcctcctccagttcgacaaagccatcaacacggcgctcgccaacagagtccggaaccgggtcaacttcaggggttctctgaacacgtaccggttctgcgataacgtctggacgttcgtgctgaacgacgtggagttcagggaggtgacggacctggtgaaggtggacaaggtgaagatcgtggcgtgtgacggaaagaactcgggctccaacgctgcagagtgacggcggcggcggcggcgccgtgggcggaccacgactgtacatataatttattacagcacggttacctgtgagcgtgtgagatgacacgcggccggtctttttttattctgatggagggtgtgtgttttcagtttgtgtgtgagctggatcatctttaataaaaactttatttaaaaggc

SEQ ID NO: 109 - DNA, 634 bp, Oryzias javanicus
cagaggtttaacatcacagcagaacatggcgctgaccgacgcggacgttcagaaacagatcaagcacatgatggcgttcattgagcaagaggccaaggagaaagttgaggaaatcaattctaaggcagaggaagaattcaacatcgagaaaggccgcctggtgcagactcagagagtgaagatcatggagtacttcgagaagaaggagaagcagatcgagcagcataagagaatccagatgtcccacctgatgaaccaagccaggctgaaggcgctgaaggcccgagacgacatgctgacggatctgttaaatgaagctcgtagaagactcgcagagctcgccaaagaccctgagacgtactccaagcttctggacggcctgatccttcaggggttttaccagcttctggagccgaaggtgaccatccgctgccgccagcaggatgtggaactagttcaggcgtcagtcaacaagaacatctccgtctataaagaggccgtgaagagcagcatcaccgtcaggatcgaccgggagcgtttccttccatctgacatctgtggaggaatagaagcgtgtaacgataacggaaagatcagagtttctaacactctggagagtcgacttgaacttctg

SEQ ID NO: 110 - DNA, 867 bp, Oryzias javanicus
gccaacactagcttctctctggatttgttcaaaaagctgagcgacaaagacaaaacggcaaatgttttctactctccgttcagcatctcttcagctctggccatggtgatgctgggagccaaaggaaacacagcctcacagatgaatgagagcctgaaacttctggactccaaggatgatgtccatgccagcttcgctcacctgctgagtcagctcaataagccgaaggccagttacgctctgagtgtggctaacaggctttttggagagcagacctaccagttcatcgaggacttcctggggaacaccaagaagcactacagcgcagagctggaaaaggtggacttcataaatagttctgaagcagccaggctcaatatcaacagctgggtggagaaacagacgcaaggtaaaatcaaagacatccttgtcgagggcgtgctgaacgacctgaccagacttgtgctggtcaacgccatctatttcaaggggaactggaacaagaagtttgatgcaaaggaaactcgtgacgctgagttcagactgaacaagaacactaagaagactgtgaagatgatgtaccagaaatccaagtttccgctgaccttcatccccgaaatcaactgccagatccttgagatgccctacaaagacaaggagctgagcatgcttgtcttcctgcccactgacatggaggacagcaccaccggcctggagaagctggagaaagccctgacctacgagaacttcatggagtggactcgtccagacatgatgaatgaggtggaggtcagggtgggcttccctcggttcaagatggagcagaaatatgacatgaagaacgtcctggtcagcatgggcatggag

SEQ ID NO: 111 - DNA, 773 bp, Oryzias javanicus
gcagctggtggccagcagagtgctgaacagcccaatgtgaaatgggacaatatcttcaggttctgtgcataggatccactgtgagcggtcagacaggtaacctttgtcactgtctttaaatggcgtcgtccacatcagcaaaaatttgcagtagggcccgttcttcaggcccagcaccgccacgatgaagctgtacagaccgccggcgacgcccaccgcagcaaaggcgatggacaagaacatcccgcagcggtttccacagcaaccctgttttccagtcaagtgaatgtaaagagccgggagcagcaccattatacctcctccgatgactccccccatgtacttcacctcctcagtaatttttccatcactgacgtacttggtgtctcccgcagggaaaaacagcacaatgttacagatgatggaaatgaccaccagtgggtacaggccgatagcgatgaagcgggagcattttccagtgcacatgttgagcagcttttttcttttaaatgtaggttagagtttcctgttctcagagggagcttccacctgaaagtgcagaaaaccaacatcaaaatgaaactgtacctcgcagttgccgtgctgatgctggcctttgtggcttacacagaggctcaggaggaagatcagacctacaccaagataagagatcagctcacagaatttggcaagacgtttgctgagaaggccaaaaccacatttgataccatcccaactagtgaatttgcagagaccaccaggaactggttcac

SEQ ID NO: 112 - DNA, 778 bp, Oryzias javanicus
tgattctgcaacttttttgaaagcgaaattgagtcttttgttcatgttgcattgagattactcttcaccaatagtttattattactttgcggcttattttgttcacctgactgaaggaaaagtgcagggctcggagcggctacttcagagcagaaagtcggtttgttgtcggggcagttgtttctgcaggaatgtcgcagaaagaaagacccaacttttaccgacaggaggtcaacaagacaatatgggaagtcccggatcggtaccaaaacctgtccccggtgggctctggggcttacggatcagtttgttcggcctacgatacaaagacaggactaaaagttgctgtgaaaaagctttctcggccgtttcagtccatcattcacgctaagagaacctacagagagctgcgcctgctgaaacatatgaagcatgaaaatgttattggcctcttagatgtcttcacccctgccacttctctaaaggaattcacggatgtgtattttgttaatcatctaatgggcgcagacctcaacaacattgtgaagtgtcagaaactcacagacgacatggagtgatgagatgagatgagatgagccaaactacttcaaacgaggcctttaaacgtgacatgtgcattaaatgtaaaactttctggcacacgccagactttatcctatttttttagtctttgtactttcagaggaggctatcagtgatttaaatgtgatgaaattcatattgatacaaagacagcaactataagagctatcatttt

SEQ ID NO: 113 - DNA, 848 bp, Oryzias javanicus
gactgttatgtaaaggttttatttccatgtcactctagcactacagaagaaaagcattgtgggtgaaatgttcacttctttgctgcctccttctggagcttcttcaaccagagcgtgatgatcttgttcctcatcctcttggccttcatcactttgtagcggtcaaagtcggacattttggctctcttctgtcttgcttcgatcttcttggcccagttgctctgcgcccatttctcgttgacttgagctttctcccaggctttcctcacaaacttctgccgggcgctgtggggcactttgatgacatagtctgtgagctgcatgcacttgaagggcatggcttgcctcttcactccagagcagggtccatccacaagagccctgttttggtcaatgacgtctatgatggcgaccagcttgccagcatgaggtccgaaggagacgtaggcgacacggccgatctcgacgaagcgggtgaacaccatggcggcttttcttccgtcggcagaaaagccacgtcgtatttatagtttttaaaagttacctttgttgttcacattatgcctggatattccgacagagatcgtggcagagacagggatagagacagaggttatggcggaggtcctcgttttggtggaagtcgcggcggcggtgggggaggtggtggaggaaagtttggtaaccccggagaacggcttcggaaaaaaacactggaacctggacgaacttcccaagtttgagaagaacttctaccagcagcacgccgatgtggcccggagaccactgcaagaagtggaacagtacagaagaaccaaggtgattacggtgaaggggagagattgtcc

SEQ ID NO: 114 - DNA, 831 bp, Oryzias javanicus
attgctgaagatgaaggcgtgtgtgctggtcctggctttgtccctggttctgaccgtgtgcgtggcccggtctccctaccagacggttctgcagcacagccggatccggggccggacgcaggggccaaacgtttgtgccatgctgcaggttaaagactcaggcaagaaatatttcaccaactgcaagcagtggtaccaccggaagatctgcagcaaacccacggtgatcgtgtatgagtgctgtccgggttacgagaagatctctggagagaagggctgtcctgcagcgctgcctctggtcaacatcttcaacactcttaaagtcgtgggagccgacaccactcagatgtacacggagcgagccctcatgagggaggagatcgaaggtcccggaagcttcacgttctttgctcccagcaacgaggcctgggccgccctgccagcagagatcctggacgcgctggtgagtaacgtgaacatcgagctgctgaacgctcttcattaccacatggtgaaccagaggctgacgtcggaggagctgaagcacggctccgtcttcacctccatgtaccaggactcaaccgtgcacatcaaccactacaaaaacgggatcatcacagtgaactgtgcacgtctcatcaaacctgatcagcacgccaccaacgggattgtgcacgtggtcgaccgtgtcatcacagccacctctagtgatctgcagaccttgattgaaattgaggacgacctggaaacgttaagaacagccatggcagccgcaggtttcaacaccatgctggagctggagggtgaagatcaatacaccatttttgcccc

SEQ ID NO: 115 - DNA, 476 bp, Oryzias javanicus
aaggttcacaagcaagatgccaggcgttacagtgaaagacgtcaaccagcaggagtttgtccgtgccctggctgcttttctgaaaaagtcaggaaagctaaaggtgcctgactgggtggacctggttaaactgggtaagcacaaggagcttgctcccagtgatgagaactggttctacatcagagcggcatccactgttcgccacctgtatcttcgaggaggtgccggcgttggttccatgaccaagatctacggtggtcgcaagaggaacggcgtgtgccctgctcaccacagtgttggctccaagaatgtggctcgcaaggtgctgcaggctcttgagctgcttaagatgattgaaaagagtcccaatggtggccgcagactaaccccctcgggaaccagagacctggacagaattgctggacaggtcgcagctgcaaacaagaaatctgcttaataaatttttgaacaaagtc

SEQ ID NO: 116 - DNA, 772 bp, Oryzias javanicus
acacctttatcactcctgtctggatttaaaccactgctttagaaatgaccacagctggaagggtcattaaatgcaaggcggcagtagcatgggagtacgggaagcctctggtgactgaggagattgaagtggcccctcctgaggccgacgaggttcgcatcaagattgtggcgactgcagtgtgccacacagacctgtaccacctgtttgaagggatggaaactgaactatttcccacaatcctcggacatgaggcagctggcatcgtggaaagtgtcggttctggtgtcaccgagtttcatccaggagacaaggttattcctctgttcatcaaccagtgtcgagaatgtcgcttctgtaaaagtcctaagaccaacctctgtgacaaaggagaaaatggtggacacgacgagttagcatttgaaaagtcaagggtcacctgtaaggggaagaagttgatgccattcatggggataggctccttctctgagtacatcctgatgaatcagactgctgtggctaagatcgacccggcggctcctttggacaaagtctgcctcatcggttgtgcgatcagcaccgggtttggagctgcagtcaacaccgctaaggttgaagcgggctccacgtgtgcagtctttggtctgggagctgtgggtttggctgcagtcatgggatgcaagtttgcaagagctgaacgaattatagctgttgacaccaatcctaaaaaagctgagaaggccaaagtttttggtgctactgactttgtcaa

SEQ ID NO: 117 - DNA, 720 bp, Oryzias javanicus
aaagctccgctgtttattgttggagtgaatatttagcatcagtaacaactacagtaagtgcatcagcagacatgacagtatttcctcctgtatcattcagcagggaacaagtacatgaaaatttgattttttattacattttttcgacattcacaaaaaaaaaatatattaaaaccgtcacagtacagtttttccggtaataatattcacaaagaaaatccaggactggaaaggaaaaaggaaatatacttaatgatcactctgcaaagaattaataaagaggaaacactgacagaacttcagttactctgatgcattttaagacagaaaagatgatccatcaaaactgtggttgggttttgaaaatagtgaatgacttatgggttcacttcatgttagcgacaaaatcctcgcccttcttgatggaggccttcagctcgccgatggcatcagccacaagcttctcctcgaaggcggacaacttgcccagccccaggttcttctcgatgccgttcttgcccaggaggagaggtgtggagaagtacttgcactcagtctcttcagacctgacaaatgagcactccaccacaccttcttttccattcatggcatccaggacagagaaggtgaagcgggcgccggcgtaggccatggagagggtagctgagccagctcctgccttggccttcaccacttctgtacctgcttcctggattctgc

SEQ ID NO: 118 - DNA, 815 bp, Oryzias javanicus
catgcttgggattttggttgccgttgccgtgtttttgcagacggtctctgctgcctctctcggtgtcgtgaacaccgagggagggagggtggagggcaaaaccatccgcctggggttatttcgtaccatggacgtcttcaagggcatccccttcgctgccgctcctggacggtttgagaagccgaggcctcacgatggatgggacggtgttatgaaggccactgagttcaagagcaggtgtcttcagctggaaattaccttggttggtacctatggcagtgaagactgtctgtacctcaacatctgggttccccacggctcctcagtatccaccaacttacctgtcatggtctggatctacggtggcggtttcttggctggagctgcatcaggtgccaacttcctggacaattacttgtatgacgggcaggaactggcggacagaggaaacgtcatcgtggtgacgctgggataccgtgttggaagcctgggcttcctgagcaccggagactctgaacttcctggaaactatggtctgtgggaccagcaggctgccatcgcgtgggtgcacaggaacattcggtcatttggaggagaccccagcaacatcaccgtcttcggagagtcagcgggtggagccagcgttaacttccagattctcactcctcacaacaaagggctcatcaggagagccatctcccagagtggggttgccctttgcccttgggcaattaaccacaacccccgcaagtatgctgaggaggtcgccctcagagtcaactgccccaccgattccaggatggctgcctgtctga

SEQ ID NO: 119 - DNA, 870 bp, Oryzias javanicus
tcaccccagccatcaaggagaaggctttcaacttcctgaccagtggttaccaaagacagctgaactacaaagattatcaaggagcatacagcacatttggaacaggaccaggcaacacttggttgactgcttttgtagtaaggtctttttctaaagcggagtcattcgtctttatcgacccaagtgtgctagaaagatctacgatgtggctagtaaacaaacaaaaaccaaacggctgctttgagaagtctggcaaactttttaacaacagaatgaagggtggagtgtcagatgaagtgactctcagtgcttacatcactgctgcattcttggagcttgagacattcaaaaataactcagtggtaactaggagcctctcctgcctgaaaggatccattaacgagctcaacaacacttacaccacagctctgctagcatacgtcttcacactggcaggagacacagagactcgaactcaccttctgcagcaccttgacagagtagcagttaaaaaaggaagtttcatctactggactcagacttcagctgaaacatctgactctctgtctgtggagatcagtgcttatgtcttgatgtctttactaagtagctcccacactgctgaagatctggggtacgcctcaagcattgttaggtggatgactgggcaacagaactattatggagggttttcttctactcaggacacagtggttgcccttcaagcgttggctctttatgctacagttgttttcactccagagggttccagcacagttacagttcagtcccctagtgcccaactagagtttgatgtgaacccagacaacaaactgctttaccaagagaaggtgatgaatgatgcaaaag

SEQ ID NO: 120 - DNA, 697 bp, Oryzias javanicus
tttctctgaacgtctttgccgcgacgcctgaagaatgtcagccgctggttacgccggtgtctctggccgacccttcagtgattcttggaaaggcatacctcctggtaggctacaccgattatgatacttttaaagtcgtcctgaaggctacagagagctcctggctgaacatcaccgagtctccgtccaacgtcacctcagaggtgttcatgtctcaggagaacaggataaacggaacctgttcggggtcgacggtgaggatgaccctggaaggaaaccaggcaagagtggcgtttacagaaatggaggcggcgatgtctgtgctaccgagctgcgacggctgcttcgtctttcacatcaactatacggcctctgatgtgaaaaagctcctcgaacccatcaatgttagccacaaagatgtggcgaatgaggctcggggtcgcagtctgtatcttttgggcagacagttgacgctcagcccctcagatctggagcatttcaggaagcaagccagctgcctcggcttcccagaagaaccggattttctcttcaatccagaaaaaagtttttgtcaagaaggggaaggaacccgaatacattacgcccaataatttcatctattaaatgtatctctatctgagagtcaatgacaaatgttcctgaacataaaaagctttttcacagctcattccatcaa

SEQ ID NO: 121 - DNA, 795 bp, Oryzias javanicus
acctcttcaagcaggccgaccgatctctgagcaagctccacaatgagtagctattcaatcatgcagatgtcctcatctggacggtcctctggttcaggaggctttggttcaggcctgagccagatatgcttccagcgtctacggtggctcaggtggcagaggaaccagagtctctgcttcctctgcttcctttggggctggaggtggtctcggttcggccgcttttgctttcggctctggtggaggtgcaggcggggctgcagagtttcaagtcggagccaacgagaaagccaccatgcagaacctgaacgaccgtctggccagctacctggagaaggtgcgcagcttggagaaggccaacgctgagcttgagctcaagatccgaaacttcctggacagcaaggctgcccccaccggccgcgactacacaaagtactacgtcaccatcacagaactccaggcaaagctcatggatgggtaccgcttaggtggaagccttcagatttctattgacaacgccaggctggcctcagatgacttcagaagcaagtatgaggctgagctcgccatgcgccagtcagtggaggcagacatcgctgagctgcggcgagtgcttgaccagctgacccttgcccggtccgacttggagctccagattgaaggcctcagagaggagctggtcttcctcaagaagaaccacgaggaggaaatgatgcaaatgaggactcaagtcactggccaggtcaatgtagaggtggacgctaagccacagcaggacttgagcgtcgtcatggc

SEQ ID NO: 122 - DNA, 868 bp, Oryzias javanicus
ggacatctacacaagcttcactcatttctgaaagtcaggttaaacacaaggagtgcgcacttacctcttgaatagagcagtgtgaatggtttcaggaggatgatactctcacatgttaagtgttcattaggacattttgcccgcagcatacccatagaaaatctctgcatggacatttgcgctcctctggctcaccaagtggtctacgatcctaaagttccatgcagagcaccggcgttccccgtacgggtttgctcatgtttctcctgcagacgtcccgtttccacccgttgctccacaacatttaccagctgactttcatgaaactgtgctctaattcttgcatcttctcatgcctgatcacatgtaccaatgcgtgccaaagaggagcagagcgacacagatgtacaaagttacggtcactgttgtaatgtctctgtctgatgaagcttttggcatcacactgtctgtggctcaatgtgactgaatcaaagtctgacttcctttttatgactgcaaacttttgagtaagaactcgaaataaactgtagatatttgtacaccaaaaaaaaaaaaaaaaaactcgagacatgaaattcgtggcccttgctcttttgctggttgtcggctcccaggccgcttccctgcaggctgatgccccctctcagctggaccagatccggtccgctgcagatgtctacatgactcaggccaagcagggtttgatcaaagccctggatcagctggatgactctccataccaggagctcaagaccaccctgactcagcgtgtggaggacctgcacaccaacatcaaggcccttcaggcccaagtgtccccttacaccgacagctttgtcagcaccgt

SEQ ID NO: 123 - DNA, 758 bp, Oryzias javanicus
tttcattgacactcccaagctttacaacccaaattcaactctggaaggaaaggtcagagccgttcactacaatgacactcccatagttggaattcagttgcatgtgttttctggctggatgtggtcgcctacagagctgaccagagttacgaccgataccaatggaaccgcacctttctcaattagtacaagttcatttgctggtgccgtcaacctccatgtaagcgacaaaccaacgctggaacaccctcaatatcgagaagcttttaaccagttaggtttacacaccgttaccatggccacgcctccctcccctgacgctgcaacagccagctctctggcgatcaagagggaggcatcgctgtcttgcaacaaggacgaggagatctttataaagtacacctttgtgagagaggcccagggctcagtggatgtcatgtttctggtcttatccaaacaaagaatttacaaacaagaatataaaacgatccaggtgcacgatgtgccagtgaatcagggagaagtgtcattcacattgaacgtatctcctgacatggcgccaaatgtccagattgttgcgtacgccattctccccagccaggaattcattgcacacgacgttttactgtccactgaggactgcttcaagaatgaggtgtcggtagagttttccccctcttctgctgtcccaggagaggaggctgaaatgcagttggctgcccacccctactcgctgtgtggtgtcagtgctatagatc

SEQ ID NO: 124 - DNA, 722 bp, Oryzias javanicus
aatctctttgtactttcagtgaaagaataatcaacatgtgctgtgaattcttaagaatttatttataactattaggtattaagatgatatttaagataattgatcttgtggttaaatcagaatattcaaggtaatttatacagatgacctgttaatgtctattttatgtcactacatcaaagatgcttcaatcatttctaatgtttcttactccatcaggactgtttgctggaatattaatgatgatttttctgtttttacaggacaaatccaccttaaagcagtgactttattcagatttattttttcaacaactttatttgtggtctagcatgtttttatcaacaagatttatcgcctttaagataaaagaatgcacacttcaacaatatatgaagtttttttacacaaatttggaacgattagcattagctaagaaattagcaattttattaaatgaataaaatacaaagtatctgctgtttgtttatatttttggatgtttttttatcacattaacagtagtatagtgtaatatttcctcatcagataagaaaactattgtttttgagttgataaaagcatgcttgagttttcaggagtgtttttgtattgttaaggatacctggatgcagcagaatggacttttttttaaccttttttttcccagcagaagtgctcccctgatcggttgacgggctgtcgcctccacattttaaccc

SEQ ID NO: 125 - DNA, 426 bp, Oryzias javanicus
caagactgatctgactgagaatcttctccaacatccaaagatgaagagcttcattgctgcagtttctctggctgttcttctgacatttgtctgctttcaggagagctctgctgttccagtcactgaggtccatgagctggagggaacactgactgatgacagtccacttcctgctgagctcaagatgccagtgacctcatggaagacgctgtatgacatcaggaggaagcgtgaggccagagctgcaggctgtcgtttctgctgtggctgctgtgggatgagtggatgtggagtgtgctgtcctttctgagatgtgctgcttctatacttggatggtccaccaaatcaacctttgtcaagcaaatgtgcaacaaatgaatacatgtacaataaacatgtacaataaagttcagtttctcacaaatg

SEQ ID NO: 126 - DNA, 778 bp, Oryzias javanicus
ggaacgcttaaatgcaggagtgtattttctattttacacccttgcaggttccttacccctacttgtagcccttctcttactacaaaactctgcaggcagcttatcgtttttaaccacccaattctttcaaccgcttgaaatgtcttcccaagccagcaagctatggtgggcaggctgcttaattgccttccttgtaaaaatgccactatacggtgcccacctctgactgcctaaagctcatgtcgaggcccctattgcaggatcaataattcttgccgcagtactcctaaaactgggagggtacggaataatacgagttattattgttctagacccccttacgagccagcttagctacccctttattgtactggccctttggggagtcgtaataacaggttcaatttgcttacgacaaacagatcttaaatccctgattgcatactcttcagtcagccacatgggacttgtagcggctggggtccttatccaaacaccctgaggttttaccggggccctagtcctaatgattgcacacggcttaacatcgtccgccctcttttgccttgctaacacaaactatgaacgaacccacagtcgaaccatacttttggctcgggggctccaaatagtcctccccctaatggcctcatggtggttcatttccaccctagcaaatcttgcactgcccccgctcccaaatttaataggagaactaataattattacctcactattcaactggtctttctgaacactatttttaacgggggccggg

SEQ ID NO: 127 - DNA, 796 bp, Oryzias javanicus
ctcgcgcctgccctcacgagatgctttccgccgcgtggggatgcggggcccgtgcttagtgcctccatcacgcacccatcctccgtgacagcgcccccaaccaccatctccccctccctcccgtggatccgcggggcgccgctcccacggatgcgctctccggtgatccacgctgaccggcccggcgtcgctggtccccgtatcggcgcggagaggatagtagttcgcgcacccgccgctcgtgtccggggcttttgaagccaacaagaatggggagcacaacctcatgctgcgtgtccgccagtcccaagttccgcagaaatgcgcactccaggctggagtcggaccagcaggagtcggatctgagcagggaggagacgggctgcaacctgcagcacatcagcgacagggagaacatcgacgagctgaacatagaatacaatccttcagaccatcccaaggcaagcactatcttcctcagcaaatctcagaatgaggaggggccacacagaaacgtgcaagacaaacgaaaaagcctcttcatcaataataaatcagaggttcctctggactacaaccagcacgacccggaccagaagcagatctatcggtttgtcagaacgctgttcagcgcggcgcagctcacggctgaatgcgccatcgtcacactggtttatttggagagactcctgacttatgcagagatcgacatttgtcccggaaactggaagaggatcgttctgggagccattcttctggcctcaaaggtctgggatgaccaggctgtgtggaatgt

SEQ ID NO: 128 - DNA, 788 bp, Oryzias javanicus
tatattttaattgcataagacaatagaaatgaccaggacaataactgatacagacattttaaccaacaatgaaacattgatgatcaaaatcaatactaaatgtgtttaacagcttttcaataatttgatttcaatctgaagaatctaatctgcatacaaaaattcagcattaatgattcgctgcacattatgagctttcgaggccggatgaatcaaaccaagctgtttgaaagcctttttacacacaggtgggtaaactaatcacaccctcaatgcactgctgacgcagccccactctctcatcatgtctttttccatctttacatatgaatgtaaggtgatgttcatgtgggcaatatatcttgtcaaaccaaggttttcgagactgaacattacgtttgtccatctcttctttagtcacagtgcacggtttgatgcatctaacatgtccagtccattgtccattcctgcaggttcgtcgtggccctccttccattacatagtagtttggacaaatgtattcaacagactcaccctcttggtattcggttctacttcccattacatctccaaattcaattgcaggtggagctgaacataggccaacatctccacatcttggaagttgattactccagtctccactttccaaacattcaatctctgcatttccttgaaggaattttccacgttttttgcattcaaactttaacttttgtcccttcttagctcttctttcctccacaggtgtcatcaagcgtagggtttgtggtattacaggcatcgca

SEQ ID NO: 129 - DNA, 734 bp, Oryzias javanicus
aatgtaagaaggccagtctttaagacaacaaaacctgaagaaaaaaaaaaactgtcctcccaataagcgaggattaaactaaactactcattccttcactctcgctagatttcttccacataattagccggatagaggccgatgtgtccgtccttaaggcgacctttgcaccaaccctgatcgtcctcttcaccgattttggtaaattcgtcaccggctttgaagctcagctcgtcctgctcctgcccttcatagtcgtacagggctttgacaggcaccgatacgagcggagaggccggctcttcctcaaacgggtttccgtcgccgttggcggaaaaggggtttccgacggtgtcttcatcggaccagtcgggggtcttttctgtactgctcgcactgctgctagtcttggtgggctgaacgggctggtccgccccggtttgattgatgcccgtcagtgtgacgccttcgggtttcttcttttcccgcctgctgagggttctgttgatgtctatggaccagtcctcaaactgaggccagttcatgggcatgccggggccgtgattgaaccggaaccatttcaggtcctcttcagcgtcggtggcagtgagagtatcttccaatgtttggtagatggtctgaaacctgtgattggttgagaggtccaggtgctgcttcacctccagaagcagctctttgaaaaagcaaattcttttgtcttcaaactgctgcag

SEQ ID NO: 130 - DNA, 922 bp, Oryzias javanicus
tcgcacctcagtaaacagcacaggcattgcatagtggaaattcacaccagagagcagcagagtcttcaaagcttttagaccataagcctgaacagaaggtccactggcaagctcaatagcccgttcaaccatgggtttatcaatgttggcaaaggcaatgtctcgtccaaacagcttgcagtagattgaaaacaaagctttgttgttgggcaaagatctccactgagaaagagccttgatgacacgtttcatcttggtgattctgtcagcgttgtcaagaattgaagggtttttcagaatggtctcttgcagtccatcggttctcactccaatctccaggacatcagcagcagctccagaaatgaaggctcttgtcttggcaacaacagaccttggcaggaaggtggcagcatcgttgatgtagaaagcggtagcagcagcacccagcataaaagaactgctataggcttctccatagatggctttgctataacgtaggctcagtctgtccagctttgggctcaagatcttcatggcaacattacaagctgcagccactgatggatgaagaacggaatcacttctgctcagggacttcatgtgagagtatgtgaagctggcaacctgcaaattctcttctgacttaacagcgttggcgagggtggaaatcagtgccattgaaggcttggtctcaaacagaacaatgcatgagagcatacgaagttctgaatcaagagccttatccatgtagagctgaagagccagttcctggaccattcttggttctgttttagcaatgttcctcagagccatgatggcttcaatatggactctgattggcagagatgcagcagcagtgccgtgaataggcataatctttgtgattgacttgaggctactggggtgtcctgcatttcccaaa

SEQ ID NO: 131 - DNA, 851 bp, Oryzias javanicus
gctcctaaataaagaatgatgggatgttttagatggactctgtggataacactcgtcttgatctgtagaggtgagcacttcagtttgaggggccggtctttgatggtttgtggtcgtgtacggtttgtgtgcagatgtcaccagcactttaagcttttcggttcaagtgtcttcgcaggaatctgactacgactatgactatgaagaaccaacaaactgctcagtggcagagaaaatcaaaggcggacatgtcacatactcacagggaggcctgccgggcagtctgctgacctatcactgcgggccgggtcagtacccatctcctgttagctccaggctctgtggcgatgatggagagtggtcacccatgaggttagcctctggcaggcttgcatcaagtgcttcttgcaaaaacgtactgtgtccaggtcagctccagctggacaatggggatttttggcccagaaaccagtggttccgcgttggaacgacgcagagctttacttgtcaagatgggtttagcttgtacggttcacctgagaggaactgcactgtctctggagactggacgggaagcactcccatctgtgacaatcatggagatgactgcgatgacccagggattcccccgggagctgaaaggtcaggaggtcagtttgacaggggagataaggtcacctaccagtgccagacgggtctgactctactcgggtcggctgagagggtttgcatggcgcaccgggagtggagcggctccacgccacgctgtcaaggcccgaacacttttgactctcccagcgatgtggctgcagccatggcaggatcattggcaggaatgatggatgtggtct

SEQ ID NO: 132 - DNA, 904 bp, Oryzias javanicus
tcgttcttagaggagtcgcctggcggtatccgcacaggcgtccaacggttaacttgcgtcttatttctgctgccttccgacgccgggaatggaccctgcgagtcggtaccaagtgttgcacaatgaagatgacgggtccgaggcttccaccagtgaggagcagccgtccacatctgcggccaccagtgcacccgcacaggaccagaatcacgctcagcccccagctttagcggcgggagaggcagcaacaaccagttctgtgggggaggcagagatgccaccccctccttatgcctccatcgacctgggagcagctgccgcagcacctgaaacgtgttttcaaggcgactttccggtgcctccaccctacagcgttgctacttccctccccacgtatgacgaggcagaaaaggccaaagcagcagctatggctgcttctgcagtggaggtgatgcccagggacgatgacttcccacccagagacgatttcagtgatgcggatcaactccgagttgggaatgatgggattttcatgctagcttttttcatggccttcctcttcaactggatcggcttctgtctgtccttctgtctgaccaacaccatcgcaggacgctacggtgccatctgtggctttggcctctccctcatcaagtggattctgattgttcggttttctgactacttcaccggctacttcaacggtcagtactggctctggtggatcttcctgcttcttggactgttgctgttcttcagaggcttcgtgaactacttaaaggttcgcaacatgacagagaacatggccacttctcacagaacgcgtctcttcttcctctactaaaggtttgcacagcattcctttccaatgtgaaccccctctcccctcgaatgacctgagcaa

SEQ ID NO: 133 - DNA, 863 bp, Oryzias javanicus
tgatttgatgctagccaaaattcccatcagctgagctggatcacacactgccaaacaaaactttctaatacttgattagtttgtgtagttctcttcagtgtccgttctgaggccacaggttattatgtctggacagtaatttatttttttcccccaatttatctttaatttccaaaaataaaatttccttaaaatcatagttttttggtaactttagtttttaaaattttaactaaaacagctgtgcatgttgttaatttttcctaaagtatgagtgggagatcattggttatgtatagaatattttattatatgactctagactctgtaaatctatccatgaaatgttcagagcttttatgttttaaactgctgatctttataaaatctgtaggcgacggaggcctctggtaagactactgaggccctggaacagctggctaaagagaaagcagctttgatgaaggagaagagtgaagcccagactttactggaggagctccagacctccaagcaggaaatgcagactcggctggaaacgattatgaaagagaattccaaatctaaagaagatcttaactcatccaaagagcaactttgcactgaattagaaaaaaccaagagtctatcccagaaaattaaagagcttcaagaagcagtttcagtcaaatcagagtccatgcaaacgctgcttgctgaaaataaaaaactcagtcaagagctgaacagcaatcacaacgagcagaaggatcttcttaagatgaaagaagagcactcaaaactcactaaagaggtggaagagttgaaacaaagcgaaagaactttgaaggatcagctgaacagggagaaagctgctgtccaac

SEQ ID NO: 134 - DNA, 818 bp, Oryzias javanicus
atgatttgaagcacatttttaattgaatgtactcaattaaagcaagtttaaacacaaagttgacaaaagaatcacttatacttatgtattttacataagttaaataagttttaccctcaaagctttttacaataatcgtaatacctgcttttacataaggattaaacaaatcaaaataccaaataatttttgaacattagattatatttcaggttatcatgtttttaccattttttctttttcatgaaatctgtgattccgtatcgttttgctcaattccatattccatacttcagtttttgataaactgtaaaaatataaacattatcaatatatacatcaaaatagaacaaaatataacatttacattgagttaatatacaagaaccacaacctcaaagttccattgtgttcctttttaaaaccttaaatcacttttttttacaccgcacattgaaattcatttcttctgcatagactaagagcaaaagtatcagaaactgctgaatgaatgcatcatatgcaaacaaacttaagtaaggtttaggaataatgataattttgcttattttatcactttctataataacagataagaaaagagagaaaaaagtctgactaataagattaaacataattataattatgaatttaaacttggtggtgagttgttcgaaatctgaggtatctatctgcaaagtttctttgcaaaatatgtctttaaaattctaaaaataaacacattcatcctttacaaggagacatattttttatatatgcacaaatgccactttaattttccttaatgactacagctt

SEQ ID NO: 135 - DNA, 777 bp, Oryzias javanicus
atccttccaggcactttctgtcaagatggccgagaacagagacaaatctaccgaccaaatgaagacgtggaaggagaacagaggttctcggaaaccagacgtcctgaccactggggctggaaatcccattggagacaagctgaacctgcagactgctggacccagaggccctcttctggttcaagatgtggtctttactgatgaaatggcccactttgacagggagcggatcccagagagagtggtgcacgccaaaggagcaggggcgttcggctactttgaggtcacccatgacatcacccgctactgcaaagccaaagtgtttgaacatatcggaaaaacaacacccattgctgtccgtttctccaccgtggctggagagtctggatcagctgacacagttcgagatcccagaggatttgctgtcaagttttacactgatgagggcaactgggacctgacgggcaacaacacccctattttcttcatcagagatgctctactgtttccgtctttcatccacacccagaagcgcaacccccaaactcacatgaaggacccagacatggtgtgggacttctggagcctgaggcctgagtgtctgcatcaggtgtccttcctgttcagtgaccggggtctgcctgatggctaccgccacatgaacggctacggctctcacactttcaagctggtcaacgctggcggcgacggcttctactgcaagttccactacaagactgaccaaggaattaagaatctgtctgtggacaaggcggc

SEQ ID NO: 136 - DNA, 858 bp, Oryzias javanicus
  misc_feature (732)..(732): n is a, c, g, or t
gtttgaagctaacggagagcttatttgggaagtttggaaagaaactcggcgccgccgctgctgctgcctccaggatgggccagcagatctcaggtcaggcggtgattaaccatctgcctgagaagctggtgaaacatgctggactggtgcgggacagtggctacctaacctacgaggagtttctggccagggtagctgagcttaatgacgtaacagccaagctagcatctgggcagcagaagcacctgctgtttgaagtgcagccaggatctgatgcctctgctctctggaaggtggctgtcaggatcgtctgcaccaagatcaacaaggacgacggcgtggtggaggcctctcggatcatgaacctgtaccagttcctgcagctgtaccgggacgtcaccagccaggccgccgaggtgctctctgcagagggagccacatccgcccagctgccctccacagactcctgccaggccagcatgtggatggggagagtgaaggagctgacggaccaggaggagtgctgcatctgcatggacggcaaagccgacctcatcctgccctgtgctcacagcttctgtcagaaatgcatcgacaaatggagtgggcggagtcaaaactgcccaatatgccgcctgcaagtgaccgctgccaacgactcgtgggtgatgtcggacttccccacggagcaggacgtggcccgatacatcctaaacctggccgacgaggcangtcaccctcacaggccttaacgcagtcacgtgacctgcgcggcgttcgacctgacacgctgaaacacagagtcactttcactgtgagaacacgttcctggtgagaaacgcgaggagacggctgcag

SEQ ID NO: 137 - DNA, 768 bp, Oryzias javanicus
tcaacctttatttcaattatatatatataaaaaaaacatgtcagcggtacattctgcagaaaattcatctgttcattcattttctttacaggctatttcctgtacagagtcagttgggttgcaggaacctctctcctctagtgtgaaggcagggttcatcctggacagtcaaaatggttctcctggtccaacttcaacttgcacagccttgcactgtttttaacaatctccacaacagaaagtgctggtatcttatctttgggttatttttgccatttaggtgtgagctgattttactgtggtctaattgatgctctggttaatagtctgtttatgtttcattcaaagcatttcattaggttcctggactggaagcgttgctactgaactggtttaactgctcagacaattaggaccactgaggaccttctctctacaacatcacgcctcattggctctcagtctgtagcggaagcgtatatcagtggagggcagcaggatccccagtccagctcgactggggtccagggcaggtctgggctgcccctcctccagctccatttcaaagtagagcagcagcagggacaagaactgtttgatctcattgacagcgaagtatctccccggacacatggaggagccagaaccgaaaggcatcaggtagtaccggagcttctgcccatctttgtagaagtctgtcttctctgcgccgtcctgaacgaagcgatcaaactgaaatgtctgaggatcttcatagatttctgggtc

SEQ ID NO: 138 - DNA, 806 bp, Oryzias javanicus
ctagctaaagcggagccgtacaccccccagtgtattctgtcacgagcccacattaacaccatagaaaactgacggtacacccgccgtcctcacaagcacgggaccaccaaaagaaccccccctcccgatctgaagtgaggaaacaaacatggcgacgtccagctcctgcgttgtgatcagtgatgaggagcagggctacgacctggatcttttctgcatccccaagcactacgctgccgatctggaaagggtgtacatcccccatggtctcatcttggataggacagagaggctggccagagagatcatgaaggaaatgggggggcaccacatcgtggccctgtgcgtgctgaaaggcggttacaagtttttcgccgacctgttggattacatcaaggctctgaacaggaacagcgaccgctccattcccatgacggtggacttcatccgcctcaaaagctactacaacgaccagtcgactggagaaatcaaagtgatcggtggagacgacctgtctactctaaccggcaagaatgtcttgattgtggaggacatcattgacacaggaaagacgatgaagacgttattgcagctcctcaagcagtacaaccctaaaatggtgaaagtagcgagtttgttggtgaagaggacgccgcggagtgtcggctaccggcccgactatgtaggatttgaagttccggataagtttgtggtgggatatgctctagactacaacgagtattttagagatcttaatcatatctgcgttattagcgaaacagggaaggagaagtacaaagcatgag

SEQ ID NO: 139 - DNA, 663 bp, Oryzias javanicus
ctcattcttgaatattttaataggataaactcaaaacaataaccaaacaaacatcatcaaaaacagtaattccataaaatatttctccaatgcaagattttagcaaatattttgataagaatgttcagtcgtttattttggctattttagtttcagacttgtaatattatacaggacagcattaggcataactcatttatatggtaaaataaggatcatattgcgcaaatcgacaagatgttcctatgaagtttttttcctgttagtatccgaagctgtgtttatgaagaatgataccatataacatcacttgtttgttcataagaaaaacatttaaaaagacaagagtttctgttgattttgagaatacgctcgtgggtggaccttcattcagcgtggagtggcgatgagctcatacctgggaggcacgttggcaaggccgctgtactctggaatcaagttgatgctgtcaggatcgttgggggaagaaaagttgaagttttgcaggagagacactataaagacaaacagctccatccgagccagagactctccaacgcaggctcgtttccctgcagaaaatgatacaaatgcagggttcttcttgaagtttccattctggtccagaaagtgttgggggttgaaggaccagggagttgacca

SEQ ID NO: 140 - DNA, 834 bp, Oryzias javanicus
gatcctctcttgcctcgccttcattggtgctgcccatggttgtgggaggcctgccatctcccccatcgtgaccggttactctcggattgtgaatggcgaggaggcggtgcctcactcctggccctggcaggtgtccctgcaggattggtctggattccacttctgtggcggctctctcatcaatgagaactgggtggtgaccgcagcccactgcaacgtcaggacttctcactacgtcatccttggagaacacgaccgcaactccaacgctgagaacatccaggtgttgaatgttggccgggtgttcaagcaccctcagtacaactcctacaccatcaacaacgacatcacgctcatcaagctggccacccccgcccagctcaacacccgcgtctcccccgtgtgtgtggctgaaaccaacgacaacttccctggaggcatgatgtgtatgacctctggctggggtctgacccaccactccaatgccaacacccccgctctgctgcagcaggcctccctgcccctcctgaccaatgctaactgcagcagctactggggcagcaacatcacagatctgatgatctgcgctggtgcctccggagcctcttcatgcatgggggactccggcggtcccttggtctgccagaaggacggagcctggactctggtgggtattgtgtcctggggaagctcaacctgcagcacctccatgcctggtgtgtacgcccgagtcactaagctgcgtgcctgggtggaccagaccatcgctgccaactgaagcgcccagttcccctccagcaccccgatcccattcagaacctctacatcaggaaa

SEQ ID NO: 141 - DNA, 857 bp, Oryzias javanicus
tcacgtcgaggaaatgatgcagtgatggccagggggacgttcgcaaacattcgcctctacaacaagttcctcggcaagcaggcgccgcagaccgtccacctgccgagtggagaaacgctggatgtgtttgatgctgcagagaggtaccggcagtccagtgagccgctgttggttctggcagggaaagagtatggctcagggagctccagagactgggcagctaagggtccatttctgctgggagtcaaggcagttctggctgagagctatgagaggatccaccgcagcaaccttgttggaatgggagtaattcctctggagtttctcccaggagactctgcagacagcctgggcctgacggggcaggagcgctacaccatcgccatcccagagcagctaacacccaggatgctggtggatgtgaagctggacactgggaaaagattccaagtgcgcatgagatttgacacagatgtggagctcacctacttccaccatggaggaatcctcaactacatgatccgcaagatgttatgaaactaaacccaccattcacacagaaaaccgctgacaaaacactgtctaaaaggattgaattattttccagaaatagaaaacggtttctcagcagtaacacagattacgggctatctttagagttataccctgagctaaatgtggaatgtaggatagaacagttccactgaaggaagctgcaagttttgttgtttaattgaatccttttcacaaagtttaacctgcaaatgggagcaatgcccttccagacaagcagcagcttgtcagtcagtctgactgttgtttgtgccacatggtggggcatacaggaaaagagtggc

SEQ ID NO: 142 - DNA, 804 bp, Oryzias javanicus
caaaggacttagaacttaaaggaaacttaagggaaaatgtaattttaaaagaaaacttacaaggcaaacataaacaaagactcagagaagacttaaaggttttgttagtcaaaatcatacacacacaaaagcagtaactcactcagacggctttattgcattcagtctgtttgaggtagtctcacacacacaaacacatccacgtcacgtgcacgcgcagcacctcactcattgacaggctcttttacattcactctgtccggacagtagcacactctttcacccttttatttttattttatgagctcagatgacaacaaattattcttattaaactcttttttactctcaatttgtcttaaccattaattttgtacttttaatcacttttattttgtacttacacgcgacgcttcttttgttcagttcacttcttttcctccttcaggaaactttattctcttttatctcactttttaacttcttatttgttcaactgctcaacgataagcacgctttacacggtcattactgttgattagtcgatacaaaaacactttcacttttccccgtgtgaaaaaaacggtcttttcggttcgatccagttaagtgaagattatttcacttcccattaacagacaacctatttagaactaagcacttgtccagaccaccaccatgaaattcgtggcccttgctcttttgctggttgcgacaacccaggccgcttccctgcaggctgatgccccctctcagctggaccagatccggtccgctgcagatgtctacatgactcaggccaag

SEQ ID NO: 143 - DNA, 777 bp, Oryzias javanicus
aggtctcaaaatagtttttaattacagcaaaaatctgacaagaatgcatgtactatacatgtaggagatccttaaaaagaacagaacagtgatcatttaaacttaagaaattacaaaataatgaacaatttcatttcaggtttttattgtgttgtgtaaaagtgtaaaagttgcttattttcacaagttataaagaatttacatgttcagtttcatattaatgttacggttcgtgtcaaaataatttaacttatttcagattcattgctcatataaaatcaccagatgtaaataatttcaagtgacttacagattttaaagttttatttatatcaccaacatttgttcagaagaatctaatcaaaaacccctttccagtccgataacaagactaaaaactttataatttaaacttcactgaaaacaaattatgaatccaaactgtgtaagtggatccacaaaggtacaatatttattttattgcaaacacattttttattttattgttgtttggtttatttgtgcaggtatgcatcaagccacagctctccagaagtcctcagagacctgacaacatccacaatcccttgccataaagatttgacctggtaagggatcccatatttcttacaaagaacacgaacttgtggggcaataaggtggtagttgtggcggggcatcgttggaaacagatggtgctcaatttgaaaattgagatgtccactgaaccagtcattgaagagggactgctcaatgttacacgtggactgcaactgc

SEQ ID NO: 144 - DNA, 794 bp, Oryzias javanicus
tgaataaattcgaaagactggttggcaggccgcctcacgaagggctgctagctggtcatcgaagcccccccgcccgcagtgggcaccgctgtgtcgctgacaacactaacctctatgtgtttggaggttacaatcctgactatgatgagtcaggaggctcagagaacgaagactatccgctgttccgagagctttggaggtaccacttcgccacaggctcctggcagcagatcagaacagaagggtacatgccaaccgagctagcttctatgtcagcggttttgcatggtaacaacctcctggtgtttggcggaactgggatcccttttggcgaaaacaacggcaatgacgttcacgtttgcaatgtcaagtacaagcgatggtcgctgctcaactgtcgaggaaaaaaacccaacagaatctacggacaggcgatggtcatcatcaacggcttcctgtatgtgtttggcgggacaacaggttacatctacagcacagacctgcacaggctggacctgacgaccagagagtggattcacctcaaacccaacaatcccccggatgaccttcctgaagaacggtataggcacgaaatagcccatgatggacaaaggatatatattcttggaggaggaacttcttggacttcttatcctctggacaaggtacatgcatacaatttggaaaccaattcctgggaggagatttacactaaacctcatgaaaaaattggttacccggctcctcggagatgccatagctgtgtacaaatacgtgacgatgtgttcatctgt

SEQ ID NO: 145 - DNA, 759 bp, Oryzias javanicus
tcagcttcgtgttcccctcacaccgcacacgctggtcccgcagcattttgccccgcaacctcagagatgggagagcgcgtgagcgaacacaccgcgcgcctgcagcagcaggaaagggagatccagagtttatcggccgagatcgagagtctaaagaacccgcagaacctcaacctgttgccccgtctggatgagctgcgggaggagaacgccaaactgaagtaccgcctcaacatcctcaagaggagtctgcaggacgacgcccccccctgcagtaaatccatgaccaacatcaaccagcggctgcagcacgtcttcggcgccgccatccgcacgtccttcccagagctggacaacccgccgctcgccgtcacgcccaaccagcaagccaagtttggagactaccaatgcaacagcgccatggcgatcgctcagatgctgaaggcgaaggggctgaaggtgagccccagagacatcgcagagaagatcctccagaaccttccagacaaccagctcatccagaggacggagatcgccgggccaggattcatcaacatccacctgaagaagaactttgtgtccaaactgttgagcaacctgctggtcaacggcgtgcagccgccgcctctggcgtccaggaagaaggtggtggtggacttctcctccccctacatcgccaaagagatgcacgtgggtcacctgcgctccaccatcatcggggacagcatgtgtcggctcttcgagttcctgggctacgac

SEQ ID NO: 146 - DNA, 716 bp, Oryzias javanicus
agtggatatcttgtcaatctcaatgccatgttggactggctctcttggcttaaatggatcagcattttcagatatggattaaatgctgcattcatcaatgagatgaaaggacaagtgttctacagtaacaacaccatcttcccaggagagttgtatttggtgagcatgggactggactactctgtttggggtttctggcagaacatcgttgctctactgggaatcatcatcatctgcatgatcttggcctacgtgcagctgcgacgggtcaaccgctggaaatgaaggacacccatctgcttgcatcgcccagcatcaattctgcactttcactttattttgacaagtaaaacaataatagagagggaaaaacatgtttttgcattggtaacgtatttttaaattctacaatcttcctatatgctcaaaagcacatgtctgactggctgatgctctttcaccaggcacacatgctgtaacatttgaccaggcgataagctgtcactaagtgccgttgacattgctttagtgagtgtctaatgaggggaacaccagtgtctaagtccttacaaagctgcttatagtgaatgagctaactttggctgatgggttaaaaaaaatatataaacaaattctcccttccacaggcagtctcatgataaattaaaaaggtggatcatgttttttttttttttggccactgttaactgctgcta

SEQ ID NO: 147 - DNA, 484 bp, Oryzias javanicus
gagtttgacagagaaggacaaggcggcggtcaaggcgctttgggccaaggttgccaagtccgccgacgcggtgggggctgacgctctgagcaggatgcttctggtgtacccccaaaccaagacctacttctcccactggccagaaatgaaacccggcggcgagtccgtgaagaagcacggcaagagggtgatgggcggggtcgctctggccgtgtccaagatcgacgacctgaccaccggcctgctggagctcagcgagctgcacgccttccagctgcgtgtggatccggccaacttcaagctcctggcgcactgcatccaggtggtcctcgccaacatgttccccaacgacttcaccccggaggcgcacgtggctttggataagttcctagccaacctggctttagctctttctgagaagtaccgctaaaggttctgcagtccagaacatgagaacccaaagcagcaaataaaccaatcaatc

SEQ ID NO: 148 - DNA, 850 bp, Oryzias javanicus
ccaactccgagtaacgcaacacgcagaaagtgatccaacaagtttgcggctgtgcaggaagtgacggaaagaaagatggagcactccaaacagagcaggtacggctccccggaccggaaggacgccgccgccgccgccgccggctccgtcatcaacggccactttgacgggctggtgaggcgcgtgagcggcgccccgcaggcggcggagcccggcgcgcgcagggccgcctccgccgtgatggagagctggaaggaggagcggaccaggagcgtggaggacaacgagatgagcctgccgtccctggccgccgcctacaccaccatcctgcgggggctgggggaggacccgcagcgccaggggctcctcaagaccccgtggagggccgccaccgccatgcagttcttcaccaagggataccaggagaagatcatcgacgtgctaaatgacgccatcttcgatgaagaccacgacgagatggtgattgtcaaagacatcgatatgttctccatgtgcgaacaccacctggtgcccatcgttggaagggtccacattggttacctacccaacaagagagttctgggcctcagcaagctggccaggattgttgaaatctacagccgcagactacaagttcaggagaggctgaccaaacaaattgctgtggcgatcacagaggcccttcaacccaccggcgtcggagtggtggtggaggcaacgcacatgtgcatggtgatgcgcggcgtccagaagatgaacagtaagacggtcaccagcaccatgttgggagttttcagggaagatccaaaaacccgtgacgagttcctgactctgatccggagctgaggag

SEQ ID NO: 149 - DNA, 814 bp, Oryzias javanicus
cgctgatgtccaccaagcagaaactcgctggttagctgccgtgtgaagcgtgtctgctgtaaaattagaaacactgtttttattttcgcgactgcaaagacgtgttcaggtttcgggtcagaggaaccaggaagttctgcagaaccagcggaggccttaaggctttcgtcgttcctcggagtgttaatctgttttaacagaacccgtctatcggaacaaatagaggagagcctcgtccatttcgacccgagcgcaaaacatgtcgaaggctccggattctccgtccagactccggtccagatcccggtccaggtcctattccagatccagatctagaagccattcccgttctaggtccagaaagcgccgctacagttctaggtctcgctcccgttcccactctccgtcctacagaaactatccctcccgggactatcagagcaaccgaggaggcttcagaggctacaaccggggctaccgccgcccgttccacttccgcggccggaaccggggcttctacccccgcggccactaccaaaaccggggaggcggcggctatggctacaagtctaactggcagggtggcggcgggggcggtggcggaggtggtggcgggggcggcggaggttggcacgaccgccgccacgaccaggagcaccacagtccgaggagggggcgctcccgctcccgcacgcccaagaagcgctcgggcagccggagccgttcccgctactcggaccgctcctcgtccggacggtcccgccactccaggcgttccagctactcctccaggtcccgctcctcgtcgccacgg

SEQ ID NO: 150 - DNA, 872 bp, Oryzias javanicus
gccgctgacgcctggggaacgctcggaccacccaacggctctggagagtgcaccctgggaggcctggggtgggtgaaagatggcagtcttttggaaagctgtagcaggtggactgctcctcactgtcggcctgctggggggaccagctgagggccaggggaatgaacggggtaagaaacaagtgtcgggcaattccgtcttcttcctgagtcaggtcagagacctctggaggccgctggtccacacagcagtcaacctctcagaaataaacagcatcaagtcgtccttccagtttcggacctatgaccccgaaggtctgattttctacggagacactaaaaacggagaggactggttcgtcctgtccctgatgaacggcatccctctgatgcaaatcagcaaagagggcatgctggtcagcgtggaaggcgggctgaagctgaacgacgggaagtggcacacgctggaagtaagcaaccaggaaaagtttgtcattctggaggtcgacggttcaactggactaaaggtggggatgcagtcccagcaaacagaggaagtcatgtcaggaggacttcgactggccgtcggtgggatcctgatcgaaaaggaaaagttgatcgttcagtttaagccacagttggacgcttgtgtgcggcatggcagctggctgaacatcagcgttccatgggaggcggaggtggaggagctatggccatgctatgaaaacattcgacccggcagctacttcccggggacaggattcgcagtgtttaacacttcagtttttcaaacagatgaagatggagagctcagggttcagctttgggcagatttccgtcagatgaatggaaccattctgagcataaagcctt

SEQ ID NO: 151 - DNA, 878 bp, Oryzias javanicus
tcagacagaccgagtgacagcatctgagtcccgcggaggaacaccccggagcctcggtggataatctctgcggagcagacaaaatgccaaacaaaaccaagaaggagaaggaaacgggcaaatctgcgaagagcggcaagagcagcagcagcgggaaggatgccgcctcggagaactctgatgagggcaacagcaagcgctccagcaacagcacgccccctcccacccagctcaacaagatcaagtactccggggggccccagctggtgaagaaggagcggcgccagagctcgtcccgcttcagcctgacccggaaccgggagctgcagaagctgcccgccctcaaagatgccccccccgtggagcgagaggagctgttcgtgcagaagctgcgtcagtgctgcgtgctcttcgacttcgtcagcgacccgctcagcgacctcaagtacaaggaggtgaagagggcggggcttaacgaaatggtggagtacatcacgcacaacagcgacgtgctgaccgaggccatctaccccgaggccgtcatcatgttttcagtgaacttgttccggacactccccccctcctccaacccgaccggcgccgagttcgaccccgaggaagacgagcccactctggaggcagcctggccccacctgcagctggtgtacgagttcttcctgcgcttcctcgagtctccggacttccagccaaacattgccaaaaagtacatcgaccaaaagtttgttttgtcgctcctggagctgttcgacagcgaagaccctcgagagagagacttcctgaagaccatcctgcaccgcatctacggcaagttcctggggctgcgcgcgtacatccggcggcagatcaacaacatcttc

SEQ ID NO: 152 - DNA, 629 bp, Oryzias javanicus
tggaaccttcccgcctgttctggtgggccgtctcggatcagaaccaggcctttggtggtcctgtcgccgtgacgatgttgaccccaccccgatcacgccgctgtggtttgtctgttgatcatgtgatgtcatccgaaagcttcagtccgaaccgccgtgaccgggtcatgtctaactccgcccccgagccagaactctgaggagcttcatcctggttgtagatcagcagtactgtgacccccccccccccccaacccctccccctgcagtgctgcgacgcgtttaaagactgaacactgaactctggatctgctggtcaaactttaactctaacttagtcgcctccgtgctggcccggcccgtttggaccaacacgccgcaagattagaaaatactgtgatgcaggctggtgtgtgcgtgtgtgtacgtacgtgtgtgagtccgattttgttttgaataaaaaggccaaatggaaaaaaaaaaaaaaaaaaaactcaagactagttctctccagcaggaaaccgcgcaccttttctaaaacaaaagaaagatcatccattcaaccggcaacagctgaaacgccgtggctgagctcgggggttgtttggataatcagctggccaggtccc

SEQ ID NO: 153 - DNA, 662 bp, Oryzias javanicus
gccccaaatacaattttactgagcacattgaactcatttgagccttaaaaatgtctgtaaactgcaaaacataaataaaaaaatttcaaaaatcaaaaaaacaaacatgtctcccttaaaaatgaatgcaaaggaaacgtaaactaaaggaatggctttttcaagaaaaaccttctgatttgcttttatttgcccaaaactttatccaaattatttctgaaggcatccaaaaaatccaacgggttgacgtaatgttcattccgcttgcagttagctaaaccatgaatgccccccgcaaaatctttggtcatcccccccttttccccagtttccccacaaaccttctccaaagtttgggcagagcaacagacttcaaaattgccctcccgcttcccccggggttcccgcccccccccccacccaaaaatactggcaatgggggtggggctggttggtttacccctctggggttcacggtaatgcctggggacggggccctgagcaacctctgcctcaatcgtcttaccatcaggacacaccaccactgacgtcatgaaacccaaaaacccaaagccctgaacaaaaatgtctgattggacatctccatcatagtttttacatgcccaaacaaatccccctgaaaacttgaggacctgtgcccc

SEQ ID NO: 154 - DNA, 658 bp, Oryzias javanicus
caaaaaccatcctctctctcatgactcgggaaaaacaccctcaagggaaagtgttgatcattggagggagtattgcaaacttcacaaatgttgcagctacttttaagggcattgtcagggccattaaagattatcaagagcctctgaaggagcatgaggtgaaaatatttgtgcgacgtggaggacccaactaccaggaaggcctgagagtgatgggggaagtggggaagacgacgggcattccgatccacgtgtttggaactgagacccacatgaccgccattgtcgggatggctctgggacacaggcccatccccaacctgccccctatggctgctcataccgctaacttcctgctcaacgctagcaacaacaccgtgactccggccaacacgagaacagcttcattctctgagcccaagacggctaatgatgtgagccctgcaaaaaagtccaaagcaggacttccagcagccaaggcaaccacactgttcagcaagcacaccaaggccatcgtctggggcatgcagacccgcgccgtgcagggaatgctggattttgactacgtctgctctcgggaagaaccctccgtggctgccatggtgtaccccttcactggagatcataagcagaagttctattggggtcacaaagaaat

SEQ ID NO: 155 - DNA, 857 bp, Oryzias javanicus
tcagccacttcagtctcatcctcacagctccggtggatttcgggagtcaaagctcatttcagcggctcttcgtccaacttctgagaacaggctccccttcagcagcaccatgaggtcagcctgtctcttcctgcttctggtcaccgcctgctgggctctgcccttccgccagtcaggattcctagactttatgatggaggatgaccccggatcggggatgactgaggtgcccggaggaagtgaaccctctgtcccaaatcaaggacccaagtgtcccttcaggtgtcagtgccaactccgggtgatccagtgctctgatctcgggttgaataaagttcctgaggatatcccagatgatacaagcctgctggacctgcagaacaacaagatcactgaaatcaaggagaatgattttaaggacctaaaaggactacaaaccctgattttggtgaacaacaaaatcaccaccatccaccctaaagcgttcagctctctgggcaagctccagcgcctctacctgtcgaagaacttgctgaaggacgtgcccgccaacatgcccaagagcctccaggagctccgcatccacgagaacgagattacaaagatcaagaaagcctccttccagggcatgtctaatgtcatcgtcatggagcttgggtccaaccccctgaagagcgcaggagttgaagcttcggcttttgccgacctgaaaaaggtctcctacatccgcattgctgacaccaacatcactgagatccctaaaggtcttcccagctccctctccgagcttcacttggatggaaacaaaattaccaaagtgacatcgaacagcctaaaaggcctgaagagcctggcca

SEQ ID NO: 156 - DNA, 711 bp, Oryzias javanicus
cacacttgcaatccaagggtgtaggagtcatgaaggtccttgctgtgcttgtccttgccgtttttactggctgcaatgccaacctcttctatgctgatgctccaaagcctcagctggaagtgctgactgatgctttctgggactatgttagcaaagccacccagactgctgatgacacactggagatgatcaagaagactcagtttgggcaggaagtcagtgcccgactgacagaaagtgcgaccctggcaaacgaatatgcaacctccattcacgagcagcttccccccgaagccaagcagctgatcaccaaagtcaccactgaggctgacgtgttgagagagcgcgtgaaccaggagctgaccaaagccagggagaacctggagccctacgccgagaacttgagatctcagatcgagcagagagtggagcagctcaaacaggagctggcaccatacaccgaatccctggattccgagaccctgagagccaccctgatgcagaagagcgaggagctgaaggccaacctggagcagagcgtgcaggacctgcagacccagctgggaccttacaccgaagacctgaagcagaaggttgaccagcacctgcaggatttcaaggagagagtggcaccaatcagcgtcagggtccagagtgagatcggtcagagagcccagcaagtggaagaaatggcaagcccctacgttgaac

SEQ ID NO: 157 - DNA, 803 bp, Oryzias javanicus
aaacatggctgctaaaggaggaaaacagagccgctcgggtctgtcgggtaggaaaggcgcaggtgagcaggatgaggaggatccgccgctgcaggccgttttagtggctgacagctttaaccggaggtttttcccgataaccaaggaccagccgcgggctttgctgccgctcggtaattctgccatgattgactacaccctggagttcctcacctccactggagtccaggagacctttgtgttctgctgctggatggccagcaccatcaaggagcatctactgaaatcaaagtggtgtcgacccacctccccaaatacagtccacatcatcacctctgacctgtaccgctccctcggtgatgtcctcagggacgtcgacgccaagtccctggtccgatctgacttcgtgttagtctatggagatgtggtgtctaacatagatgtcagccaggcgctgcaggagcacaggcaacggcgaaagatggagaagaacatctctgtgatgactatgatgtttaaggcgtcatctccaggtcacagatcccgctgtgaggaagatgacgtcatcgtagctgtggacagcaagagtcagcaaattttacactaccagaagacgcaaggcttgaagaagatgcagtttcccatgaacattttccattgtggaactgatgagtttgaaatcagacatgacctcctggactgtcacatcagcatctgctctccacaggttgctgagctcttcaccgataactttgactaccaaactagagatgattttgttagaggaatactggtcaacgaag

SEQ ID NO: 158 - DNA, 510 bp, Oryzias javanicus
gaagaagcttttagacatggctccccgcaaaggtaaagaaaagaaggaggagcaggtgatcagtctgggtccacaagtggccgagggcgagaatgtgtttggagtctgccacatcttcgcctcttttaacgacaccttcgtccacgtcactgatctctccgggaaggagacgatctgccgcgtgactggagggatgaaggtgaaagccgacagggacgagtcgtctccgtacgcagccatgttggcggcccaggacgtggcgcagcgctgcaaggagctcgggatcacagcgctgcacatcaagctgagagccaccgggggcaacaggacaaagactcctggacccggagcccagtccgctctcagagctctggctcgttctggaatgaagatcggccgtatcgaggacgtcacccccatcccatcagactccaccaggaggaagggaggacgccgtggtcggcgtctgtgagcaggacctactctttacaataaaagtttgttaaagtc

SEQ ID NO: 159 - DNA, 590 bp, Oryzias javanicus
tcctcgtgtttttatcttcagtttaccggactctataccctggagaaatcctctgtggaccggccgtgggtgcgcgggtgtccctttgaggtgcttcctgattgaggtccagcaacgaagtttgttgccatggcgtccccatcatgatggtgtgcatgatggcgtggaggcccatccaggctctcatgtccatgtcagccaccttcaagctgttggagagctccagccagcagtggctgcaggggctggtttacctggtggggaaccttctgggctcggcgctggccatctacaagtgtcagtccatggggctgctgcccacgcactcgtctgattggctggctttcatcgagcctcctcagagaatggagatcatgggaggggggatggtgctgtgaagcccgacgaccagcccctcccagcagagggcgccgcaacccttcatatttcatcgtgttcagaacagttgcattcatgtttgtgagctgatcctggatctgctcaggtttcctcctgtgtgctttcatgctaacctgcttcattttctgtagattttttttgtgaaatgttctaataaatccataattatt

SEQ ID NO: 160 - DNA, 884 bp, Oryzias javanicus
ggagatcagcgatgatgaggcagaagaggagaaggaagagaaggagaccaaggaggaaggtgaggacaagccaaagatcgaggatgtgggctcagatgatgaagaggacgctaaagacaagggcaagaagaagaagaagatcaaggagaagcacttttcagtagaaggtcagctggaattcagggctctgctctttgttccccgccgtgcaccttttgacctcttcgagaacaagaaaaagaagaacaacatcaagctgtacgtcaggagagtcttcatcatggacaactgtgaagagctcatcccagagtacctaaacttcatccgtggagtggtggactctgaagacctgcccctcaacatttcaagagaaatgctgcagcagagcaagatcttgaaagtaatccgcaaaaacatcgtcaagaagtgcctggagctcttcgcagagctggctgaagacaaagaaaactacaagaaattctatgaagctttctcaaagaacatcaagctcggcatccatgaggactctcagaaccgtaagaagctgtctgagctgctgcgctaccacagctcccagtctggagatgaaatgacctccctcacagagtacctttctcgcatgaaggagaaccagaaatccatctactacatcactggtgaaagcaaggaccaggtcgccaactcggctttcgtagagcgcgtccgcaagcgtggcttcgaggtcctgtacatgacagagcccatcgacgagtactgcgtccagcagctgaaggagttcgacggcaagaacctggtttctgtcaccaaagagggcctggagctgccagaggatgaggaggaaaagaagaagatggaggaagataaggccaagttcgagaacctct

SEQ ID NO: 161 - DNA, 770 bp, Oryzias javanicus
ctgtgcaggctttggaccagaggacaccacgagatgaagctgctccctcacactttgtttctctgcctggccctcgctctggcctgggctgaccaccatggacagcaccaaaaaggtgaccgtggccgtgaccgtgaccgtgaccatggccgtggccgtgaccatggccgtggccgtggccgtgaccgctgtaaaggcattgagatggacgctgttgccttgaatgaagagggaatcccttactttttcaaggatgactacttgttcaaaggcttccatggacaggcagagctgttcaatgaaacttttgctgagctggacgaccatagccacctgggctacgtggacgctgctttccgcatgcactacgaggacgaccccaaccaccacgaccgcatgttcttcttcttggacaacaaagtgtttgcctattaccaacacaagctggaggcaggctaccctaaggacatctctgaagtgtttcctggaattcccgaccatctggatgctgctgtggagtgtcccaaatcggagtgcggtgaagactctgtcatctttttcaagaagaacgaaattttccacttctacgttaaaaacaagaccgtagatgagagggatttccagtccatgccgaactgcacctctgctttccgcttcatggagcactactactgcttccacggacacaagttctccaagtttaacccaaaaaccggcgaggtgcgcggcagataccccaaagatgcccgtaaatttttcatgagatgccc

SEQ ID NO: 162 - DNA, 791 bp, Oryzias javanicus
attttgtccacatcacaaaatctaaactcagagactgacacaggacacggagtggttggaaacagaaacggtgaagcccttcagatgtccttcagcacttcctgtccacgtccgtcactccaggtgggaggccatgttttcttgatgctgggtctgtccttcagcttcctgaagtaagccgccacgttggggtaacgctcctcgctccctctgaaggaagcaggagacgtggtgaaacttttgtatcaaggtttgactgtgtgagtaaaactaaatgatttggagaacttacccgttttggaagaaatacgcaaaaactggaaaaataatcacatcggccaaagaaaaagactttccagccaggaagccactgcatccctggaagaagaagaagaagaaatgatctctgcaacatgagatcagtctgaacatggatgcaactcgggctgtgtgcatccagaacttctctgaagaagacaacaccatgggtttgtgactacagttgtgcaaacacacacgccgctgatgcagcttcccgcctgtttcctaaactgtgtcgcaaccgagggcaacttctgctttgaagctgcagcttcactgaaacgtctcaggtctgtctggtgttacactcacatttactaaactgtgatgaacatcagcttcttcttgtttttccagagacactttctgcctttctctaattttaatcaataaaaatgtgtaataataatactgaagcaaaaacacagtcaccatctataaggttaagtgtttctattcttatataatat

SEQ ID NO: 163 - DNA, 761 bp, Oryzias javanicus
gacgaatctcctctgctggttttccagctccctatcaaaagactttgttgttgtagctctttaaggtaaggtgcactattttacagcatttttgtaccaattacattttggaactaaatagactttttttttttgtcaaatacaacattttcttatcagtcttggtcaaggactaagatttatttttatgattttgtttaactttgttgagaccattgccgcagtgctttgcatttcacagacaagatgaacttggatgagtacttccagaggattggctttcacggctcttgtgaaaaagcagatcttgcaacactaaaggtgatccacaagctgcatgtcatgacgatcccatttgaaaacctcagcattcactgtggtgagaggatctccatgaacctggagctcattttcaacaagctggtgaggagcaaacgtggaggctggtgcctggagagcaactacttgtttggatgggtgctgcgggcaatgggcttcaacgccacaactttgggtgccagcgtatatagcagtatcctgaatgcctattttccgggagagaatcatctcattaaccgagtcatcgttgatgggaaagcttacattacagacgtgagctttggagtgtcgtcccaagtgtgggagccgctggagctcatctctgagaaggatcagcctcaagcagcgggggtcttttgtttgactaacaacggagacatgtgggtgctgggaaagtcttctagaaaaccagtagtgatgaa

SEQ ID NO: 164 - DNA, 775 bp, Oryzias javanicus
cttacaggtgctcgtgctcatcatgagggttcttgccgtgatcctcgctctggctgtcctctcagggtcccacgcccgcagcgtgcccctggaggacctgcagaacacgtgggaggtgaccgtggagaagttccagaattacatcacagacctgaacacaaaggctgacggcgtgctggagaacatcaggagctctcagatcagcagagagttggaaactctgatccaggacagcatgtcagagctggccatgtacagagacgacctgcagaacaagctgggcccctacacccagcagagcgcggagcgcctgagccaggacctgcagctgatggcggacaaactgcgcgagaacatgaatgaggcgcgcgagcaggtggaccgctacgtccaggagctgcagaccatgatggagcagaacgccgacgacgtcaagctcagggtgtccacctacagccgcaagctgaagaagcgcctcaacaaggacacgcaggagatcaagaggtacgtcagcgagtacgtcgaggagctccactctcgcacctccgagaatgtgggagagctgaaagcccgcctggatccatacttctctcaggtccgggacaacgcccaggccaagatctccaccctgaacgagctgctccagtcccagatcgagaacgtgaagaccagggtgcagaccacccaccaggacgtcaaggagcgcgttaaggtcgccaccgaggacctgcgctcttctgtggagggaaggattcaggaggtgcgcaactggttc

SEQ ID NO: 165 - DNA, 788 bp, Oryzias javanicus
  misc_feature (711)..(711): n is a, c, g, or t
cgtgttcgagaccaatcctcagatgaagaagaccattgaagtcaaaaccgccccgcacgtttctgcctacaaacactctgagcacggcagtgagaacgacaaaggcgtgatgacctgcgtggctcacggctacccgctgcccaccgattgggtttggatcaaagaagaaggtggaaaccagaagcctctcaccaacgccaccagcgaccgatacgagataaagagcacccccaacaagacggtgctgaccatcaacgacctgaacatcgagacggacgccgggaactacgtctgtcacggaaccagcgagaagggcaccgacaaggacaccatctacctgcgcgtgcgcagtcgcctggccgctctgtggcccttcctcgggatcgtggcggaggtcatcatcctcgtgaccatcatcttcatctacgagaaaaggagaaagcccgacgaggtcaatgacgacgacgactcaggagctgctccactgaaaagcaattccaccgccaaccacaaagacaagaacgtgcggcaaagaaactccaactgagaaggtgtgaggtggagctgcagcgaggactcacttcctgtcgcatcaacgtgctttctgcaaagttcctgtggttcatgttagaggaaaatccttctgttcctgttttagtttttattttctttttaatcacgttgtggaaatgatgtgaatttctgcttcangctgtctggcaaatattgcatgaatcaagtgtatcaaatccctgggattctggttggaggagtgttagtttgtgatg

SEQ ID NO: 166 - DNA, 1001 bp, Oryzias javanicus
agtcgcatgctccggccgccatggcggccgcgggaattcgattagcgtggtcgcggccgaggtacaggcagatcttcagagactttggcacacactacatcactgaggccgccctcggaggagactttgagcacactatcatcctaaacaaggagaagctggtgagctcagattattctctggaggaatacaagaattgtgtgcaggctggccttaacatgggggcaaacatttatggactttacgtgtcaggaggggttgagggtggatcctgcgacgggcttctgaacgaaatgggagaggacaccaagaagggcagcatggtggaggactttgtcgctgtcgtgaggggggggagcagcgaaagcatcactgctttgctgtctaagaaacttcccagcccacagctgatgaggctgtggggggaaggcgtgcactataaccccgacttcattcgcatgaaaacgcagccgctgtatgagctggtggtctccagagacttcagctatcatgatactctcaaaagaaacctgaagagggtcctgtcggagcatctggctgaatcgagctcgtgtcgctgccctccctgccacaacaacggagtggctgttttgaaagtgacgcggtgtgagtgcgtgtgccctgtcggctacactggaaggagctgtgaggtcactcaaaggccaaaagatatctccatcgatggcagctggagttgttggggtgcgtggtcgtcttgtggaggtgggaagaagtcccggacccgacagtgcacaacccagcacccaacaacggaggacttgaatgctggggactgcagaggagtccaccaactgcttttaatcagcaaatatggcaaagatggcgaatcgtttgttaaagaacagtttttttggctgtgcacattcatttatacataaaacataagatcaaaaaaaaaaaaagctgtttactctgcccgggcgggcgctcgaatcactagtgaattcccggccgccctg

SEQ ID NO: 167 - DNA, 773 bp, Oryzias javanicus
tcagtcgtaactttgaaaaagtttttttctgtacataaatctgagaatcaagtgtaaataaatatgaaaaaataccagaatttagaattaaatcttcattgaactgatttctgaaactgatgcatgttaataaaaatgttctgacacttatagtttaaataatattttcttgatggtctcctgcagcatccgtcttcatccaggtttctctaactgttattagccaaagcggagcggaccgcctgcagctggaagaagacgcgtcctccctcctcctgacagaccgtcttcagctcctcccgactcatccccagcagcatggagccgctgagcacgcccagacagcgcacggtgattgggctgaagcctttgtcttccagccaggctttaacttctgctggtttggacttcttcgtgaggacgggagacgccaccgcctcatcgggctgctcctcctgttgacgttgttgctttgcggcgcccaattggcccacgatgttgcggcgggaacacaaggggcctgtgaatgcgcctcgtcctgtccggatgtcggcagtctgcagagggagctcagggccttgacggagaggcaaacatctgtggaagccagtctgacagacgcccaaagccagatcgccgaactgaagagcaaagcacgaaccccagtagttttttctgcaacaactgctgaaagtggagccatcggaccccttcacacccacacaaacatcctctataaaaaagcaataacaaatgtgggcggggcctacaat

SEQ ID NO: 168 - DNA, 755 bp, Oryzias javanicus
tccgaacgcggttcttcgggcggaaccggactcttgacacccgtcggacgtgtctcggctgtccggactctgtggtttttttttacgctcgctttggttagccgctttgctaacgcgaacattcgggtcgtatcagctggtggtttcgttgcgcaagaagacccagtgtgacccggtggcgcctcgctgctctccgtgatgggatccagggcgtcgacgttacttcgagaagaagaaatcgaagaaattaaaaaggagaccggcttctcccacagtcagatcactcgactctacagccgcttcaccagcctggacaagggggagaacggcgcgctcagccgcgaggacttccagaggattccggagctggccatcaaccctctgggagaccggatcatcaacgccttcttccctgatggagaggatcaggtgaacttccgaggcttcatgcggactctggctcacttccgaccggtggaggacaacgagaagaacaagaacgtctccaacacggagccgctcaacagcaggatgaacaagctgcactttgccttccagctgtacgacctggaccgggatgacaagatctctcgggacgagctgctgcaggtcctgcggatgatggtgggcgtgaacatttcagacgagcagctcggcagcatcgccgacaggaccatccaggaggcggacaccaacggggacggatgcatctccttcaccgagttcatgaaggttttggagaaagtggacgtgga

SEQ ID NO: 169 - DNA, 784 bp, Oryzias javanicus
cgcgggacctgtggagggggaacccgactgtctgacatacctgtactactctgctgctgacccagttaaagacaccagttctggtctggtgggacctcttctaatctgtcggctgggatctctgaagaaaggaatacagaaaaactacaacaaagagtttcacctcatggccacagtgtttgatgagaacctcagctggtatcttgatgagaacattcagacttttaccactgctcccaagacggtcaaaaaagaggatgatggcttcatggagagcaataaaatgcatgcaatcaatgggtttgtgtataacaacttaccaggcctgaggatgtgcaaaggggacaaagtctcatggcacctgtcaggactgggaacagagacggacatcgtcagtctttatttccacgggaaccgtttcatctaccggcagaacagacgagacaccatcagcgttttccctcacatctcacacaccgtcacaatggagccagacagcatgggtcagtttgaagtggtctctgccacactggaccattatcgtaatgggatgcgagccaactacactgtggagaaatgtggcctatttcagcgtcagggtgaggtgatgctccactcaaaaacctactacattgctgctgtggagatggactgggactactcgccaaaccgcacctgggaggcagagatgttcccggatcaaggaatgaaccctgcccccattttcattgatccacaaggaggattcataggttcccgctacaaaaaggttgtgtaccgacag

SEQ ID NO: 170 - DNA, 894 bp, Oryzias javanicus
agacagagaagaggttggaagctgagacagaaacatggcaagagtgctcgcagcggtgtgtgttctgctcactctgcacatcgcttcatctagcaagctggtgtgtcacatgaccaactggtcccagtacagacctggcagtgctgcattcaccccagagaacatcgacccattcttgtgcacccatgtcatctacgctctggcgaccatcaacgacttcaaccagatcgtcacagtagaatggaacgaccagcagatgtattcgagccttaatagcctcaagaacgcaaatcctgcactaaaaactctgctgtctgttggaggctcagtcaacggaatcagtccattcatccccatggtcgccaaacctgagagccgtgccgtcttcatcagatctgccatcagcttcctgcgcaacaacaactttgatgggctaaatctggcctgggaatatcctggacacaacggcagcccgatgcaggatagagagaggttcacccttctggtctcggagttgtcccaggccttcatagatgaagccactgaaaacaagaagacgcagctccttctttcagccaatgttgctgcactaccctcatctgtcgacaaagcatacgaggtcaacaagatttcccctcatttggacttcttgaatgtcttgacctatgacttccatggatcttgggagccagttacaggacacaacagcccattgtatcgcagtgctgtggacactgcctcacaaatgcattataacattaattcttcaatatcctactggctgtctctgggagcagcagctgataaactgctgttgggtttccccacctatggacggacctttcaactcagtggatcagcaaatggcctgggggccctggccaatgggcctgc

SEQ ID NO: 171 - DNA, 787 bp, Oryzias javanicus
accccttcttcagagccgacgcccagaagtacccctcaaagacccagcagctccacttcatcgaaagctacctgcgggagtctgaccccggcttccacaacctgagcgccgccgaccaaatgaagctgaaggaggagctgtttgtggaggtgaacaggtttgccatggcgtctcacttcttctggggcttgtggtcgctcatccaggcccgtctgtccaccatcgagttcggatacctggagtacgccaaggccaggtttgaggcctacttccagcagaagaagatctgggcggcctaaaggatccgcggcatcggctggtttgatgttgagccgacgcgtcctgaagcgggacgcagaggccgttccgccatccatgcttttctcccacccccccttcaggggatttgctgctgctgacaggcgacctgggcgctctgaggacgcgcctctgtccgctgcagctcagagttcactctgtgccagcacagactcttactaaccctcatcagcagccagaggcgctctgcttctgctttgttcaaaccacacggctaacaccacgcctcagagcgggacagctgtgcagacgctgctagtttggaaactgacattgcatcgatgtgcgcctgtagatcctctgtcaatcggaaaccaaccaaaacgaacctttgggtcgtctcactcgccgagcagcgcctggttagctgtcggggcgatgccccgcctgtaggtgaccgctctgttgtctgtaaacactcactgctagtctgactttctattcttaa

SEQ ID NO: 172 - DNA, 944 bp, Oryzias javanicus
ccgaggagagttatcaaaaactggatgctgctcctttagtttgcaggtggatttgcagcagtgaagatgggaggtggaggtcagcagacggagccggtcagcggcaaagggacaggttctttcacctgggaggaggtgcagaagcacagcaacaggaatgatcagtggctggtggtcaaacgaaaggtttacaacgtcactaattgggccaaaaggcatccaggggggtttcgtgtcatcaaccactatgctggagaggatgccacagaggtatttgctgcctttcatccagatcaggacttcgtacaaaagtttctaaagcctctccacattggagagttggcagcaacagagcccagccaggacagagaaaagaatgattccataataaaagatttcgattcccttcgagtccaggcagaaaaagatggtttgtttaaggctaagcctttgtttttcctcctccatctgggtcacatcatactgctggaggcactcgcatggctctctgttctttactgggggaccagctggacaatgactttcctgtgtgcgacactgctggcaattgctcaggcacaggcagggtggttgcagcatgactttgggcatctatctgtctttaagaagtctaaatggaatcacattgtacacaaatttgtcattggacagttaaagggagcttcagccaattggtggaatcacagacatttccagcaccacgccaaacccaatatcttcaggaaggaccctgatattaacgtgatggacgtcttcgtacttggaaacactcaaccagtagagtacggaataaaaaagatcaagaaaatgccctacaatcgccaacaccagtatttcttcctcgtgggtccacctctactaatcccaatattctacaattttaatatattttacaccatgattacccgccgtgactgggtgga

SEQ ID NO: 173 - DNA, 677 bp, Oryzias javanicus
gtttgattttgtgatggagttcctgaaaagcatggtgggtgcagagtatgtgggattcagcaatgcaaccttccagtcagaaagggagactggcgatagaaattacgcgattggttattacctaaaagagaaaaagtgttttcctaagaatgcagatatgatggcagcacttgacttctactttcagttgtgctctatagaggtcacctgtgagtcaggaagtgtcatggctgccacgcttgccaatgggggcatttgtccaatcacgggggatcgtgtgttgagtgctgaagctgtgcgcaacacgctgagcctcatgcactcctgtggcatgtacgacttttctggacagttcgccttccatgtcggcttaccagccaaatctggcgtttccggggctgtactgctggtggttcctaatgtcatgggcatgatgtgttggtccccgcctttagatcgccttggtaacagtgtgcgaggcattcacttctgccaggacctggtgtcttacttcagcttccacaattacgacaacctgaggcatttcaacaagaagcacgatcccaggagacgatcagatgatgacccaaataaatcagtggtaaacctgatgtttgcggcgtacagcggtgacgtctctgccctgaaaaggtttgccctttctgctgtggacatgg

SEQ ID NO: 174 - DNA, 723 bp, Oryzias javanicus
gagggattggagggaagcaaaggatccgaggaaatccacttcctgacacatccatcactggagctgaagactaaacagtggacgcagtggtgctcttcggtggccagacacctatgagagatcagctcttatctttaaaggattaaagcaaaaaaaaaaaaaaaaaaaatgaaggcattcagcattgcagttgcagtgacactcgtgctcgcctttatttgcatccaggagagctctgccatcccagtcaatgggcaggtcacagagctggaagaagcagcaagcaatgacactccagttgcggcacgtcacgagatgtcaatgcaatcgtggatgatgccaaaccacatcagggaaaagcggcagagccacctctccatgtgcagcgtgtgctgcaactgctgcaagaactacaagggctgcggtttctgctgcaggttctgaggggtcctgcgacaaccaccacacattaatttattattaggcttgtttttccatactttccacaagaaattcactttttttccctctttatctcagtctgtcatgtgataggatacttttattgtaatgaggaactcaagaaaacagataaaggctgttggttttatttattttctactctttttgtaagatcaataaattgtcaatttccaaaggaaattaaatgaatgaattaaatgcgtttttttttcaatgtaattattcaaataaaacagttgt

SEQ ID NO: 175 - DNA, 792 bp, Oryzias javanicus
  misc_feature (691)..(691): n is a, c, g, or t
ggacgtcctgcacaccaacacccggggctcccccgaccgcagcatcggcatccagaggcccgtggggcacatcgacatttaccccaacggaggcaccttccagccgggttgcgacatccagaacaccctgctggggattgcatcagcggggatcaagggtctccagaacatggaccagctcgtcaagtgctcccacgagcgctccatccacctgttcatcgactccctggtgaacaccgatcaccagagcatggcctaccgctgcaactccaaggaggccttcaacaagggggtgtgcctgagctgcaggaagaaccgctgcaacaagctgggctacaacatcaacaaggtccgcacggcccgcagcaccaagatgtacctgaaaacacgcgggatgatgccctacaaagtttttcactatcaggtgaagggacacttcttcagcaagagtgagcagaacttcacggagcagccgatgaagatttcactgtttggaactcacggggagaccgaagacatcgcctttgtcctgccggaactgagctgcaacaaaaccgtgtccttcctcctcaccaccgacgtggacatcggcgagctgatgatcgtcaagctgcgctgggagaaggacaccctcatcagctggtccggctggtggggcagcagccagttccacatccggaagctccgcatncaatccggagagactcagtccaaggtgatcttcaatgcccaggaaggagagtttgctgatctggtcagaggaggggaggacgccgtctttgtcaagtcaaa

SEQ ID NO: 176 - DNA, 883 bp, Oryzias javanicus
cagccattgacttttaccagaagtttggcttcgagatcatcgagacaaaaaagaattactacaaaaggatagagccagcagatgcccatgtgttgcagaagagcctgcgctccccatgtgcaccacccagtggagagcttcagaaggccgagtaggggcacaagattgtgaaagacccaaatgtgccctccagcacagaaacagaactgtgacaaaagccccaccgtcaacacatttaaaagtttcttcaaaggacacaaaaagacaaattttgacgataaaaaaatgctgcattctgtttcaaaggcaatttctgcttattttacttctcccctttctcatagagaattgcaaattccaacaagctcggtatcctaaattacccccaacggcaaaggccaaaaccaggcaattgtagatgaaatggaggataagaatgtgatgtgtttcaaaccataccgttttgttttgagtagtttttatgcagttgaagcttaaaaaggacatatctacaatgaaagtttattttaaggggtgaaaatgtttaaaagacctgctctgaaagacctagttatagctgaccagcttgaagtggtaactggaattgttccctggatcgaacaagttcatctgatttgagtttggcaagtttgttgatatgtccgcaggggaggacgagaaatgtctcctgagtattgacacaaatgagatggaacggttttgtggaggtgatacgttgtcaccatctagcttttcagattacgttggtttttttggtctgttaatttaatgcccgtcatcaactgatgtttttatttttcacatttggaaggaaaatggttttgaggttctcctgggtgcactattaactgattgatggaaa

SEQ ID NO: 177 - DNA, 827 bp, Oryzias javanicus
gggattcgtgcgagttctctgaggatgagctgcagctgctgagcagatctcaacatgaacctctgctggagagctgctttcctgctgggagccttcctcggttctgcctgcagctctgatgtggaaggatactccaaaaccgacggggcgtgggtcctctcgctgcacaggagacagtattcagtggactctgtggccgactgcgccgccaaatgtgacgctgagacatccttcacatgcaaatccttcgcttacattgaaaaggatcaggaatgttggacagcggcatcaaactccaaaacagagccggtcctgcggagacggagctcagctttgtatgagaaaaatgattatcttctggaatgtgttaatggattaggaacagattacagaggaacgaaggccaaaacaaaatcaggaaaagcgtgtcagagatgggattcccgattcccgcacagacccaacattactccacagacccacccccgggcggacctggactcaaacttctgcagaaacccagacgaggacagcggggggccctggtgttacaccaccgacccggacactcgctgggagcactgcaacgtgacgagctgcagcgaggactgcatccactgcaatggggaggactaccgagggaaagcctccatcaccgagagcggctacacctgccagcgctgggactcccagagccctcacagccacggttacagcccggacgctcttccagagaagtatctagaagagaactactgcagaaacccggacggagacccccggccgtggtgcttcaccaccagcgctgccaaacgatgggactactg

SEQ ID NO: 178 - DNA, 758 bp, Oryzias javanicus
tttaatcacaagaatgctgcggtacgttgtagccctctgttttctggcctcctccgtctactccaacgagtccgatcccttggagaacaactgccaggtcgccaatttccaagtcaagcaggactttgatagaaccaggtatgcaggtgtgtggtacgctgtagggaagaaggaccccgaggggttgttcttgcttgacaatgtggtggccgagttcaaagtggaggaggatggcaggatgaccgccaccgcaaagggcagggtcatcatcctcaacaactgggaaatgtgcgcccacatgttcgccaccttcgaggacacgcccgaccctgccaagttcaggatgaaatactggggtgcggcgtcctacctgcagaccggaaatgacgaacattgggtcattgacaccgactacgacaactacgccatccactactcctgcaggcttctagactccgacggcacctgcctggacagctactccttcgtattttctcgccaccccacgggcctgagggccgaggaccagctcatcatcacccagaagaagaacgatatttgcctgctgggcaaatacagacgggttgcacacaacggtttctgcgagaactcagccgacttgagcccacagtgatccccacctcaaggacttcgttcccctccgctggattcctctcaaccttatgagagagtcttcctttttgtgtagaaatgtgtaaattctcacggtcacttcctcacttcggtagctctccatt

SEQ ID NO: 179 - DNA, 325 bp, Oryzias javanicus
tcacacagactccttcctctacctgggtgctcattgcttgatcactagtgccgttccctccaaaaagagctttcctgctggaccacctccaatctcatttgatggtgagccgggcccctccccaagactgcataatgtgagacctggaggatctgatctgtgactgataacacctccaccattgaatgccacaatgccccaacacctgattaatgcttgaagaaaattatgtgtaaacaaacttatgcgatgttcgtggatggatgacaggtgtacacaactggaaagtgtggtctggtcccgatcatggtggagaattacgatg

SEQ ID NO: 180 - DNA, 938 bp, Oryzias javanicus
tggtgctcgctctatgtctggccctagtggctggacaaactgtcaactatgctcctgaatttactgctggaaaaacgtatgtgtacaaatatgagatgtccgtctcgagtggtctgcctgaggagggtttggcaagagctaaaatgaacggcagctgcaaatttctcataagtgcttttgacaaaaacacatatatcatgaagcttgttgatcctgtgatgcatggtttcaccggcatggggccgaaggaccagggagaacaagttgaggatatccctaaagcacttgaaccagagtggagtactcccatcaagtttgaatacaacaatggcattgtaggaaaaatctatactcccgatgggttctcagatatagtgctgaacttctacaaagccgttctgaacattcttcatcttaacatcaaaaagacacagaacatttatgatcttcaggaggatggaattcagggcgtgtgcaagacattctacactgtaaatgaagatgttaaggctgaccgcatcctcctgactaaaactagggacatgaacaactgtcagacaaggatctatagggacatggggttggcatacactgagaaatgtgacaagtgccaacaggaatcaaagaatctaaggggttcaacatcgtacagatatgccttgaaaccagttcctagtggtatcatgatcctggaggcagatgtaaatgagttaatccagttctcgccattctctgagggttacggagccgttcaaacagagaccagacaaaccttggtcttcgaggagattcaggattcccccattgcaccaaaatctgctgaatatcatcactgtggatctcttaagtatgagttctctgatgagtttgagtggacaccctttcagtttgtcaaggtcactgataaacaggcccagattgaggagcttctgaatcat

SEQ ID NO: 181 - DNA, 771 bp, Oryzias javanicus
acgacttcagcaggacaaggatggcgccgatcttcaagactgtctttgtggccctaatggtggccctcacacaggcggccccattggaggattcagcagcaggcggtggagatgccgctttgccagatcgatgtgcgggtatcgagtttgatgccatcacccctgacgacaagggtcaaacttttttcttcaaaggggatcacgtttggaagggctttgagggtgacgctcaaccagccattcagtactttaaggagttaaacggccatgttaatgctgctttccgtatgcacaacccagagaaccaaggtgaccacgaccatatctaccttttcctggatgacaaagtgttcagttactttgatcacactcttgaggaaggctatccaaaagaaatccaagaggactttcctggagttcccacccacctggatgcggctgtggagtgtcccaaaggagagtgcgtcaccgattccattttgttcttcaaaggtcaggatgtgcacgtttatgatattgccacaaagactgtgaagaccaagacatggccccaacttcctgcctgtacctccgtcttccgctggttggagcactattattgtttccatggacacaacttcacccgttttcaacctgtaaccggggaggtgaccggtagttaccccaaggacgcccgcaggtatttcatgcgctgccccgactttggtcatggaggcgaagccacaaccttgaaatgcagtgacttcaaaatggacgccatcactactgatg

SEQ ID NO: 182 - DNA, 20 bp, Artificial Sequence
  Arylamine N-acetyl transferase foward primer
cattcactgtggtgagagga

SEQ ID NO: 183 - DNA, 20 bp, Artificial Sequence
  Arylamine N-acetyl transferase reverse primer
tccaaagctcacgtctgtaa

SEQ ID NO: 184 - DNA, 21 bp, Artificial Sequence
  Apolipoprotein E1 forward primer
ggatccatacttctctcaggt

SEQ ID NO: 185 - DNA, 21 bp, Artificial Sequence
  Apolipoprotein E1 reverse primer
gtctgaacatgtttccatgag

SEQ ID NO: 186 - DNA, 20 bp, Artificial Sequence
  Basigin forward primer
atcctcgtgaccatcatctt

SEQ ID NO: 187 - DNA, 20 bp, Artificial Sequence
  Basigin reverse primer
aggaactttgcagaaagcac

SEQ ID NO: 188 - DNA, 20 bp, Artificial Sequence
  Complement component C8 beta forward primer
gtgcactataaccccgactt

SEQ ID NO: 189 - DNA, 20 bp, Artificial Sequence
  Complement component C8 beta reverse primer
agtgacctcacagctccttc

SEQ ID NO: 190 - DNA, 20 bp, Artificial Sequence
  C1q-like adipose specific protein forward primer
ctgttgacgttgttgctttg

SEQ ID NO: 191 - DNA, 20 bp, Artificial Sequence
  C1q-like adipose specific protein reverse primer
aaaaactactggggttcgtg

SEQ ID NO: 192 - DNA, 20 bp, Artificial Sequence
  Catalase forward primer
aaaaactactggggttcgtg

SEQ ID NO: 193 - DNA, 22 bp, Artificial Sequence
  Catalase reverse primer
ggatggacggccttcaagttct

SEQ ID NO: 194 - DNA, 20 bp, Artificial Sequence
  Calcium binding protein P22 forward primer
ccacagtcagatcactcgac

SEQ ID NO: 195 - DNA, 20 bp, Artificial Sequence
  Calcium binding protein P22 reverse primer
caaagtgcagcttgttcatc

SEQ ID NO: 196 - DNA, 21 bp, Artificial Sequence
  Ceruloplasmin forward primer
gtcagtttgaagtggtctctg

SEQ ID NO: 197 - DNA, 21 bp, Artificial Sequence
  Ceruloplasmin reverse primer
gaacctatgaatcctccttgt

SEQ ID NO: 198 - DNA, 20 bp, Artificial Sequence
  Complement factor B/C2-B forward primer
cgtggacatatgtgacccta

SEQ ID NO: 199 - DNA, 20 bp, Artificial Sequence
  Complement factor B/C2-B reverse primer
tgtccaaatctgttggaatg

SEQ ID NO: 200 - DNA, 21 bp, Artificial Sequence
  Chitinase forward primer
gatgcaggatagagagaggtt

SEQ ID NO: 201 - DNA, 21 bp, Artificial Sequence
  Chitinase reverse primer
agatccatggaagtcataggt

SEQ ID NO: 202 - DNA, 20 bp, Artificial Sequence
  Choline kinase forward primer
ccagaagtacccctcaaaga

SEQ ID NO: 203 - DNA, 20 bp, Artificial Sequence
  Choline kinase reverse primer
ttcttctgctggaagtaggc

SEQ ID NO: 204 - DNA, 20 bp, Artificial Sequence
  Choriogenin L forward primer
tgcaacaccaggaaactatg

SEQ ID NO: 205 - DNA, 20 bp, Artificial Sequence
  Choriogenin L reverse primer
ttttcggctcataccaatac

SEQ ID NO: 206 - DNA, 20 bp, Artificial Sequence
  Delta-6 fatty acyl desaturase forward primer
tggacaatgactttcctgtg

SEQ ID NO: 207 - DNA, 19 bp, Artificial Sequence
  Delta-6 fatty acyl desaturase reverse primer
cctgaagatattgggtttg

SEQ ID NO: 208 - DNA, 21 bp, Artificial Sequence
  Delta 6-desaturase forward primer
tctcgtgtcattagccactat

SEQ ID NO: 209 - DNA, 21 bp, Artificial Sequence
  Delta 6-desaturase reverse primer
attcttctctgcctgaactct

SEQ ID NO: 210 - DNA, 20 bp, Artificial Sequence
  Glutaminase forward primer
tgagtcaggaagtgtcatgg

SEQ ID NO: 211 - DNA, 20 bp, Artificial Sequence
  Glutaminase reverse primer
ttaccaaggcgatctaaagg

SEQ ID NO: 212 - DNA, 22 bp, Artificial Sequence
  Glutathione S-transferase forward primer
tcgacaagatggagcacaaatc

SEQ ID NO: 213 - DNA, 23 bp, Artificial Sequence
  Glutathione S-transferase reverse primer
cagtttgttcccctgagacttga

SEQ ID NO: 214 - DNA, 21 bp, Artificial Sequence
  Hepcidin forward primer
aactgctgcaagaactacaag

SEQ ID NO: 215 - DNA, 20 bp, Artificial Sequence
  Hepcidin reverse primer
ccaacagcctttatctgttt

SEQ ID NO: 216 - DNA, 20 bp, Artificial Sequence
  Leukocyte elastase inhibitor forward primer
gtttgatgcaaaggaaactc

SEQ ID NO: 217 - DNA, 21 bp, Artificial Sequence
  Leukocyte elastase inhibitor reverse primer
catgaagttctcgtaggtcag

SEQ ID NO: 218 - DNA, 20 bp, Artificial Sequence
  Lipoprotein lipase forward primer
ttcactgtttggaactcacg

SEQ ID NO: 219 - DNA, 20 bp, Artificial Sequence
  Lipoprotein lipase reverse primer
accagatcagcaaactctcc

SEQ ID NO: 220 - DNA, 20 bp, Artificial Sequence
  N-acetyltransferase forward primer
tcgagatcatcgagacaaaa

SEQ ID NO: 221 - DNA, 20 bp, Artificial Sequence
  N-acetyltransferase reverse primer
gcagaaattgcctttgaaac

SEQ ID NO: 222 - DNA, 20 bp, Artificial Sequence
  Plasminogen forward primer
tgctgagcagatctcaacat

SEQ ID NO: 223 - DNA, 20 bp, Artificial Sequence
  Plasminogen reverse primer
aatgtaagcgaaggatttgc

SEQ ID NO: 224 - DNA, 20 bp, Artificial Sequence
  Retinol binding protein 4 forward primer
agaaccaggtatgcaggtgt

SEQ ID NO: 225 - DNA, 20 bp, Artificial Sequence
  Retinol binding protein 4 reverse primer
accccagtatttcatcctga

SEQ ID NO: 226 - DNA, 20 bp, Artificial Sequence
  Ring finger protein 141 forward primer
tctgcaccaagatcaacaag

SEQ ID NO: 227 - DNA, 20 bp, Artificial Sequence
  Ring finger protein 141 reverse primer
tttgtcgatgcatttctgac

SEQ ID NO: 228 - DNA, 20 bp, Artificial Sequence
  TBT-binding protein forward primer
ccatcaatgttagccacaaa

SEQ ID NO: 229 - DNA, 20 bp, Artificial Sequence
  TBT-binding protein reverse primer
cgtaatgtattcgggttcct

SEQ ID NO: 230 - DNA, 21 bp, Artificial Sequence
  Transferrin forward primer
tcacacagactccttcctcta

SEQ ID NO: 231 - DNA, 21 bp, Artificial Sequence
  Transferrin reverse primer
tcagtcacagatcagatcctc

SEQ ID NO: 232 - DNA, 21 bp, Artificial Sequence
  Uridine phosphorylase 2 forward primer
gacattctgtaccacttcagc

SEQ ID NO: 233 - DNA, 21 bp, Artificial Sequence
  Uridine phosphorylase 2 reverse primer
ccactttgtacatggagtagc

SEQ ID NO: 234 - DNA, 21 bp, Artificial Sequence
  Vitellogenin 1 forward primer
acaaaaggttccactctcagc

SEQ ID NO: 235 - DNA, 21 bp, Artificial Sequence
  Vitellogenin 1 reverse primer
ccaactttaacctccatctcc

SEQ ID NO: 236 - DNA, 20 bp, Artificial Sequence
  Warm temperature acclimation related 65 kDa protein(C243) forward primer
cgacaagggtcaaacttttt

SEQ ID NO: 237 - DNA, 20 bp, Artificial Sequence
  Warm temperature acclimation related 65 kDa protein(C243) reverse primer
tttggatagccttcctcaag